(19) United States
(12) Patent Application Publication
(10) Pub. No.: US 2024/0218394 A1
(43) Pub. Date: Jul. 4, 2024

(54) GENE THERAPY FOR OCULAR DISORDERS

(71) Applicant: The Trustees of the University of Pennsylvania, Philadelphia, PA (US)

(72) Inventors: Jean Bennett, Bryn Mawr, PA (US); Junwei Sun, Philadelphia, PA (US); Kenneth Shindler, Havertown, PA (US); Devin McDougald, Philadelphia, PA (US); Ahmara Gibbons Ross, Philadelphia, PA (US)

(73) Assignee: The Trustees of the University of Pennsylvania, Philadelphia, PA (US)

(21) Appl. No.: 18/504,261

(22) Filed: Nov. 8, 2023

Related U.S. Application Data
(62) Division of application No. 16/607,834, filed on Oct. 24, 2019, now Pat. No. 11,879,133.
(60) Provisional application No. 62/488,989, filed on Apr. 24, 2017.

(51) Int. Cl. C12N 15/86; A61K 9/00; A61K 48/00; A61P 27/02

(52) U.S. Cl. CPC C12N 15/86; A61K 9/0048; A61P 27/02; A61K 48/00; C12N 2710/10043

(57) ABSTRACT

Compositions and methods are provided for treating ocular neuropathy in a subject. In one aspect, a recombinant adeno-associated viral vector is provided which includes a nucleic acid molecule comprising a sequence encoding NRF2. In another aspect, a recombinant adeno-associated viral vector is provided which includes a nucleic acid molecule comprising a sequence encoding SIRT1. In desired embodiments, the subject is human, cat, dog, sheep, or non-human primate.

### CROSS REFERENCE TO RELATED APPLICATIONS

This application is a divisional of U.S. patent application Ser. No. 16/607,834, filed Oct. 24, 2019, which is a US National Stage of PCT/US2018/029167, filed Apr. 24, 2018, and which claims priority to U.S. Provisional Patent Application No. 62/488,989, filed Apr. 24, 2017. Each of these applications is incorporated herein by reference.

### STATEMENT REGARDING FEDERALLY SPONSORED RESEARCH OR DEVELOPMENT

This invention was made with government support under Grant No. EY019014 awarded by the National Institutes of Health (NIH). The government has certain rights in this invention.

### INCORPORATION-BY-REFERENCE OF MATERIAL SUBMITTED IN ELECTRONIC FORM

The sequence listing labeled “UPN-17-8143.US” created Nov. 2, 2023, and which is 311,449 bytes, is incorporated herein by reference.

### BACKGROUND OF THE INVENTION

There are a number of disorders that cause visual impairment and blindness due to disease of the optic nerve. Damage and death of ganglion cell axons which comprise the optic nerve or loss of the myelin nerve sheath surrounding the optic nerve leads to loss of vision. The onset of disease can be rapid (1-2 weeks) and there can be pain on eye movements. Rapid onset is typical of demyelinating, inflammatory, ischemic and traumatic causes of optic nerve damage. This sort of rapid onset is typical of multiple sclerosis, which affects 2.5 million people globally. A more gradual loss of optic nerve function is associated with compression of the nerve or toxic, nutritional or hereditary conditions. For compressive or nutritional disorders, the primary insult is addressed. However, there are limited treatments for optic neuritis and other optic neuropathies consisting primarily of steroid administration and related treatments aimed at modulating inflammation (beta-interferons, glatiramer acetate, fingolimod, teriflunomide, alemtuzumab, dimethyl fumerate). Although some vision can return 6 months after an episode of optic neuritis, there is permanent vision loss from death of ganglion cells that is not addressed by immunosuppressants. Further, a significant number of people suffer recurrences in the initially affected or contralateral eye. It is thus desirable to protect the ganglion cells in order to prevent retinal ganglion cell death and resulting permanent vision loss.

Others have described the use of NRF2 to promote neuronal survival in neurodegeneration and acute nerve damage (Xiong et al, J Clin Invest. 2015 Apr. 1; 125(4): 1433-1445).

### SUMMARY OF THE INVENTION

In one aspect, a recombinant adeno-associated virus (rAAV) is provided. In one embodiment, the rAAV includes an AAV capsid, and a vector genome packaged therein. In one embodiment, the vector genome includes (a) an AAV 5′ inverted terminal repeat (ITR) sequence; (b) a promoter: (c) a coding sequence encoding a human NRF2: (d) an AAV 3′ ITR. In one embodiment, the coding sequence of (c) is SEQ ID NO: 4. In another embodiment, the coding sequence of (c) is SEQ ID NO: 7. In one embodiment, the vector genome includes an AAV2 5′ ITR, a CMV/CBA promoter, a Kozak sequence, the coding sequence of SEQ ID NO: 4 or SEQ ID NO: 7, a bGH polyA and an AAV2 3′ ITR. In another embodiment, the vector genome includes an AAV2 5′ ITR, a human Synuclein Gamma (human SNCG, hSNCG) promoter, a Kozak sequence, the coding sequence of SEQ ID NO: 4 or SEQ ID NO: 7, a Woodchuck Hepatitis Virus (WHP) Posttranscriptional Regulatory Element (WPRE), a bGH polyA and an AAV2 3′ ITR.

In another embodiment, the vector genome includes (a) an AAV 5′ inverted terminal repeat (ITR) sequence: (b) a promoter: (c) a coding sequence encoding a human SIRT1: (d) an AAV 3′ ITR. In one embodiment, the coding sequence of (c) is SEQ ID NO: 2. In another embodiment, the coding sequence of (c) is SEQ ID NO: 12. In one embodiment, the vector genome includes an AAV2 5′ ITR, a CMV/CBA promoter, a Kozak sequence, the coding sequence of SEQ ID NO: 2 or SEQ ID NO: 12, a bGH polyA and an AAV2 3′ ITR. In another embodiment, the vector genome includes an AAV2 5′ ITR, a hSNCG promoter, a Kozak sequence, the coding sequence of SEQ ID NO: 2 or SEQ ID NO: 12, a WPRE, a bGH polyA and an AAV2 3′ ITR

In another aspect, a composition is provided which includes an rAAV as described herein and a pharmaceutical acceptable carrier or excipient.

In another aspect, an aqueous suspension suitable for administration to a subject is provided. In one embodiment, the suspension includes an aqueous suspending liquid and about 1×10⁹ viral particles to about 1×10¹³ GC or viral particles per eye of a recombinant adeno-associated virus (rAAV) as described herein useful as a therapeutic for the treatment or prevention of optic neuropathy. In one embodiment, the suspension is suitable for subretinal or intravitreal injection.

In yet another aspect, a method of treating or preventing an optic disorder in a subject in need thereof with a rAAV as described herein, is provided. In one embodiment, the disorder is an MS related disorder. In another embodiment, the disorder is glaucoma.

In yet another aspect, a method of preserving retinal ganglion cell (RGC) function in a subject in need thereof with a rAAV as described herein, is provided.

Other aspects and advantages of the invention will be readily apparent from the following detailed description of the invention.

### BRIEF DESCRIPTION OF THE DRAWINGS

FIG. 1 provides a plasmid map of the pAAV-CMV/CBA-hNRF2-3×FLAG vector.

FIG. 2 provides a plasmid map of the pAAV-CMV/CBA-hSIRT1-3×FLAG vector.

FIG. 3 shows 3 photomicrographs of ARPE19 cells transduced with AAV2-CBA-hNRF2-3×FLAG vector. pAAV-CMV/CBA-hNRF2-3×FLAG uses the chicken beta actin (CBA) promoter plus cytomegalovirus (CMV) enhancer to drive expression of the human NRF2 cDNA with a C-terminal 3×FLAG epitope tag. (CBA) promoter. The NRF2 sequence terminates into a bovine growth hormone (bGH) polyadenylation signal. The entire AAV expression cassette is flanked by the AAV2 inverted terminal repeats (ITRs). Transfection of ARPE19 cells resulted in both immunohistochemically detectable transgene expression and production of NRF2 protein of the predicted size (bottom left).

FIG. 4 shows 3 photomicrographs of ARPE19 cells transduced with AAV2-CBA-hSIRT1-3×FLAG vector. hSIRT1-3×FLAG uses the chicken beta actin (CBA) promoter plus cytomegalovirus (CMV) enhancer to drive expression of the human SIRT1 cDNA with a C-terminal 3×FLAG epitope tag. (CBA) promoter. The SIRT1 sequence terminates into a bovine growth hormone (bGH) polyadenylation signal. The entire AAV expression cassette is flanked by the AAV2 inverted terminal repeats (ITRs). Transfection of ARPE19 cells resulted in both immunohistochemically detectable transgene expression and production of SIRT1 protein of the predicted size (bottom left).

FIG. 5 demonstrates that transfection with pAAV-CMV/CBA-hNRF2-3×FLAG results in gene activation in Neuro2a cells. pAAV-CMV/CBA-eGFP was transfected as control in a second cohort of cells. RNA was extracted at 48 hours post-transfection and synthesized into cDNA. qRT-PCR analysis of quinone-1 (mNQO1) mRNA (a major downstream transcriptional target of NRF2) reveals enriched transcripts levels (**=P<0.01) in the pAAV-CMV/CBA-hNRF2-3×FLAG-treated cells compared to nontransfected and reporter transfected cells.

FIG. 6A demonstrates that intravitreal injection of AAV2-CMV/CBA-hNRF2 in a mouse model of experimental optic neuritis results in preservation of retinal ganglion cell (RGC) function compared to controls. Retinal function was assessed in a masked fashion by measuring the optokinetic reflex (OKR), a measure of visual acuity. The greater the cycles/degree, the better the visual acuity. Sham-induced mice treated with vehicle or AAV2-eGFP exhibit robust OKR scores throughout the experiment. EAE-induced mice show a significant decline in visual function at experimental day 21 and throughout the remainder of the experiment, mirroring the inflammation and cell death occurring in the optic nerve. However, EAE-induced animals treated with AAV2-NRF2 exhibit significantly enhanced OKR scores at day 35 ((d′à: P<0.01) and day 42 ((a): P<0.05) and demonstrate a trend towards improvement at days 21, 28, and 49 compared to EAE-induced animals treated with vehicle.

FIG. 6B demonstrates that intravitreal injection of AAV2-CMV/CBA-hNRF2 in a mouse model of experimental optic neuritis results in preservation of retinal ganglion cell (RGC) numbers compared to controls. Retinal ganglion cell numbers were assessed in a masked fashion by histology. EAE immunized animals exhibit significantly reduced RGC numbers compared to non-induced controls injected with vehicle (**=P<0.01). However, treatment with AAV2-CMV/CBA-hNRF2-3×FLAG provides significantly enhanced RGC survival compared to EAE immunized controls injected with vehicle (@=P<0.05).

FIG. 6C demonstrates that intravitreal injection of AAV2-CMV/CBA-hSIRT1 in a mouse model of experimental optic neuritis results in preservation of retinal ganglion cell (RGC) function compared to controls. Retinal function was assessed in a masked fashion by measuring the optokinetic reflex (OKR), a measure of visual acuity. The greater the cycles/degree, the better the visual acuity. Sham-induced mice treated with vehicle or AAV2-eGFP exhibit robust OKR scores throughout the experiment. EAE-induced mice show a significant decline in visual function at experimental day 21 and throughout the remainder of the experiment, mirroring the inflammation and cell death occurring in the optic nerve. However, EAE-induced animals treated with AAV2-SIRT1 exhibit statistically significant preservation in OKR compared to AAV2-eGFP+EAE at day 35 (P<0.01) and day 42 (P<0.05) and compared to EAE-induced animals treated with vehicle. Data represented as mean+/−SEM. Differences in OKR compared using one-way ANOVA followed by Turkey's HSD test. Differences considered statistically significantly at P<0.05.

FIG. 6D demonstrates that intravitreal injection of AAV2-CMV/CBA-hSIRT1 in a mouse model of experimental optic neuritis results in preservation of retinal ganglion cell (RGC) numbers compared to controls. Retinal ganglion cell numbers were assessed in a masked fashion by histology. EAE immunized animals exhibit significantly reduced RGC numbers compared to non-induced controls injected with vehicle (**=P<0.01). However, treatment with AAV2-CMV/CBA-hNRF2-3×SIRT1 provides significantly enhanced RGC survival compared to EAE immunized controls injected with vehicle.

FIG. 7 are 4 photographs showing that AAV2 efficiently targets RGCs with intravitreal injection.

FIG. 8A is a graph demonstrating that SIRT1 does not affect inflammation. Scoring scale: no infiltration=0; mild=1; moderate=2; severe=3; nodular=4.

FIG. 8B is a graph demonstrating that SIRT1 does not affect demyelination. Scoring scale: no demyelination=0; mild=1; moderate=2; severe=3; massive=4.

FIG. 8C is a graph demonstrating that NRF2 does not affect inflammation. Scoring scale: no infiltration=0; mild=1; moderate=2; severe=3; nodular=4.

FIG. 8D is a graph demonstrating that NRF2 does not affect demyelination. Scoring scale: no demyelination=0; mild=1; moderate=2; severe=3; massive=4.

FIGS. 9A and 9B is an alignment of the coding sequence for native (SEQ ID NO: 4) and codon optimized hNRF2 (SEQ ID NO: 7). These sequences show 73.55% identity over the full length.

FIG. 10 provides a plasmid map of the pAAV.CMV.CBA.hSIRT1 vector.

FIG. 11 provides a plasmid map of the pAAV.CMV.CBA.hSIRT1.3×FLAG vector.

FIG. 12 provides a plasmid map of the pAAV.pAAV.CMV.CBA.opt.hSIRT1 vector.

FIG. 13 provides a plasmid map of the pAAV.CMV.CBA.opt.hSIRT1.HA vector.

FIG. 14 provides a plasmid map of the pAAV.hSNCG.hSIRT1. WPRE vector.

FIG. 15 provides a plasmid map of the pAAV.hSNCG.hSIRT1.HA.WPRE vector.

FIG. 16 provides a plasmid map of the pAAV.hSNCG.opt.hSIRT1. WPRE vector.

FIG. 17 provides a plasmid map of the pAAV.hSNCG.opt.hSIRT1.HA.WPRE vector.

FIG. 18 provides a plasmid map of the pAAV.CMV.CBA.hNRF2 vector.

FIG. 19 provides a plasmid map of the pAAV.CMV.CBA.hNRF2.3×FLAG vector.

FIG. 20 provides a plasmid map of the pAAV.CMV.CBA.opt.hNRF2 vector.

FIG. 21 provides a plasmid map of the pAAV.CMV.CBA.opt.hNRF2.HA vector.

FIG. 22 provides a plasmid map of the pAAV.hSNCG.hNRF2.WPRE vector.

FIG. 23 provides a plasmid map of the pAAV.hSNCG.hNRF2.HA. WPRE vector.

FIG. 24 provides a plasmid map of the pAAV.hSNCG.opt.hNRF2.WPRE vector.

FIG. 25 provides a plasmid map of the pAAV.hSNCG.opt.hNRF2.HA. WPRE vector.

FIGS. 26A and 26B shows in vitro characterization of AAV2 vectors. RT-qPCR analysis of relative quantities of human NRF2 or human SIRT1 mRNA in 84-31 cells treated with (A) AAV2-NRF2 and (B) AAV2-SIRT1 compared to nontransduced cells.

FIG. 27 shows AAV2 RGC transduction efficiency following intravitreal delivery. Quantification of RGC transduction (n=5 retina) following intravitreal injection of AAV2-eGFP.

FIGS. 28A and 28B demonstrates experimental groups and clinical scoring of EAE. (A) Outline of the experimental groups used in the study. (B) Clinical scores of sham (n=10) and EAE-induced (n=35) animals. Data represented as mean+SEM.

FIG. 29 demonstrates the effect of gene transfer on RGC survival during EAE. RGC numbers quantified per retinal region. Left bars: central. Middle bars: midperipheral. Right bars: peripheral. Data represented as mean+SEM. *P<0.05, **P<0.01 by 1-way ANOVA and Tukey's HSD post-test.

### DETAILED DESCRIPTION OF THE INVENTION

Described herein are several agents which can be delivered to retinal ganglion cells or other neurons to prevent or ameliorate permanent vision loss. The agents, NRF2 and Sirtuins (SIRT1, 2, 3, 4, 5, 6, 7) act by decreasing oxidative or metabolic stress and thus by providing neurotrophic support.

Sirtuins are NAD-dependent protein deacetylases. Intravitreal delivery of sirtuin activators such as resveratrol have been shown to reduce deficits from optic neuritis and optic crush injury in mouse models. (Shindler et al. Invest Ophthalmol Vis Sci 48(8):3602 (2007): Zuo et al Invest Ophthalmol Vis Sci 54(7):5097-102 (2013)). Sirtuin-1, encoded by SIRT1 (herein used interchangeably for the coding sequence and expression product), links transcriptional regulation directly to intracellular energetics and participates in the coordination of several separated cellular functions such as cell cycle, response to DNA damage, metabolism, apoptosis and autophagy. Two isoforms are known. Transcript variant 1, which includes the entire 747 amino acid protein (SEQ ID NO: 1), and transcript variant 2, which lacks amino acids 454-639 (using the numbering from transcript variant 1). Exemplified herein is the use of transcript variant 1. However, similar constructs utilizing transcript variant 2 are also contemplated herein. The native SIRT1 sequence encoding sirtuin-1 is shown in SEQ ID NO: 2. A codon-optimized SIRT1 sequence encoding sirtuin-1 is shown in SEQ ID NO: 12. It has been shown that activators of SIRT1 significantly attenuated retinal ganglion cells (RGCs) in a dose-dependent manner. Shindler, K S et al, Invest Ophthalmol Vis Sci. 2007 August; 48(8):3602-9, which is incorporated herein by reference.

Nuclear factor-like 2 also called Nuclear factor erythroid 2-related factor 2, encoded by NFE2L2, also called NRF2, is a transcription factor that regulates a pathway of genes that decrease oxidative and other forms of stress. As used herein, the term “NRF2” is used interchangeably to refer to the nuclear factor-like 2 protein and its coding sequence. It has been shown that following optic nerve injury, RGC death is significantly increased in Nrf2 KO mice, and administration of AAV-NRF2 with Nrf2 reduces RGC death in retinitis pigmentosa mouse models. See, Kimura et al, Oxidative Medicine and Cellular Longevity, Volume 2017 (February 2017); and Xiong et al, J Clin Invest. 2015; 125(4): 1433-1445, which are incorporated herein by reference.

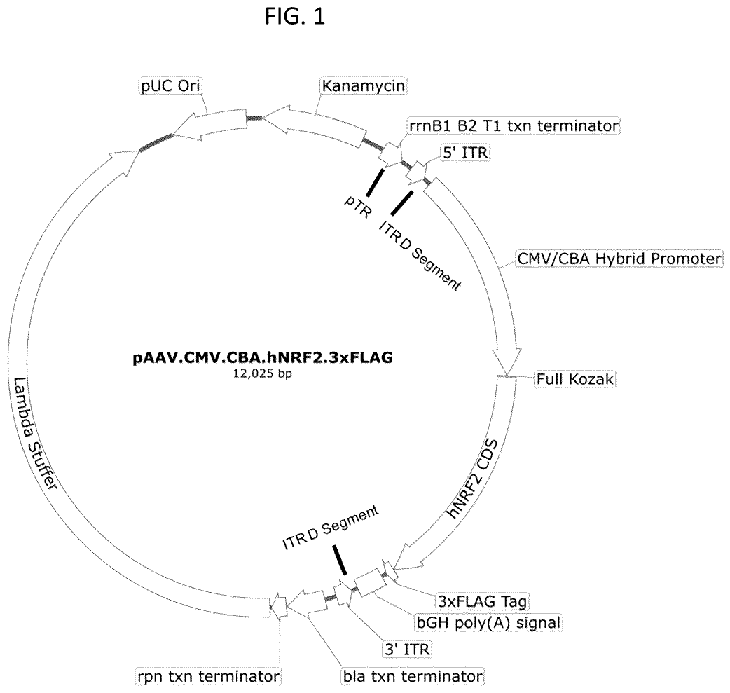

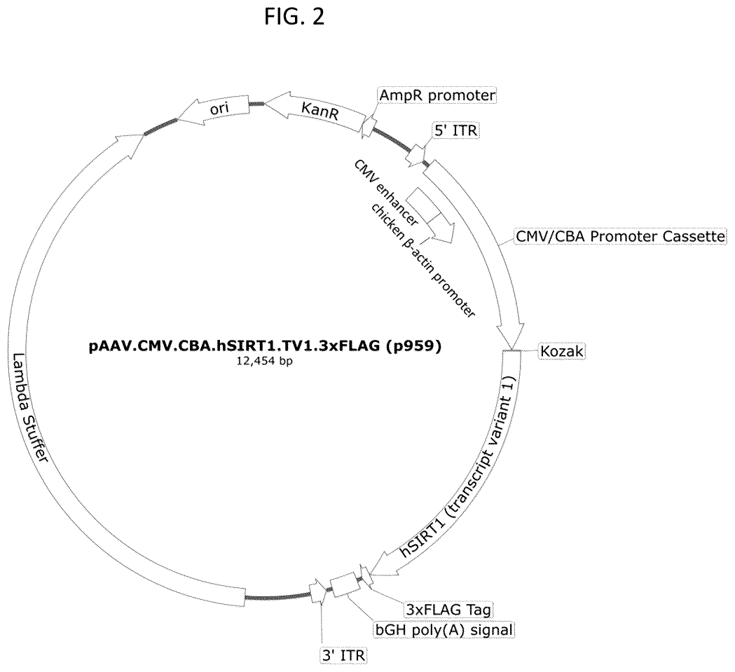

Described herein are rAAV vectors capable of delivering NRF2 (FIG. 1) and sirtuins (SIRT1, 2; FIG. 2) to diseased tissue, including retinal cells in the eye. The vectors described herein are useful in the treatment of a wide variety of genetic and acquired optic nerve disorders that affect the structure or function of the optic nerve. In one embodiment, any of such optic disorders or conditions is referred to as optic neuropathy. Such optic disorders include glaucoma, optic neuritis, optic neuropathy, retinitis pigmentosa, amongst others. Other optic disorders include ischemic optic neuropathy, compressive optic neuropathy, infiltrative optic neuropathy, traumatic optic neuropathy, mitochondrial optic neuropathy, nutritional optic neuropathy, and toxic optic neuropathy. Still other optic disorders include hereditary optic neuropathy including leber's hereditary optic neuropathy, dominant optic atrophy, Behr's syndrome, and Berk-Tabatznki syndrome. In one embodiment, the ocular disorder negatively affects the retinal ganglion cells. In another embodiment, the disorder negatively affects any of the neuronal cells.

Glaucoma is a neurodegenerative disease of the eye and it is one of the major causes of irreversible blindness. It is estimated that, by 2020, more than 80 million people will be affected worldwide, with at least 6 to 8 million of them becoming bilaterally blind. Glaucoma is characterized by damage to the optic nerve and progressive degeneration of retinal ganglion cells (RGCs), which are critical elements for vision loss. The factors associated with pathogenesis of glaucoma include high intraocular pressure (IOP), increased oxidative stress, aging, glutamate neurotoxicity, and susceptibility genes such as optineurin and myocilin. In one embodiment, the ocular condition treated using the rAAV vectors described herein, is glaucoma.

MS is a central nervous system disease characterized by chronic inflammation and demyelination. Approximately 2.5 million people are affected globally, with an average age of onset of 30 years. About 50% of patients diagnosed after 25 years require a mobility aid. The disease is largely believed to be an autoimmune condition, with infiltration of CNS by autoreactive immune effector cells. The central “trigger” for disease onset is mostly undefined, with complex genetic modifiers and environmental factors believed to play a part. See, Dendrou et al. (Nat Rev Imm, 2009), which is incorporated herein by reference. See, McDougald et al, Investigative Ophthalmology & Visual Science March 2018, Vol. 59, 1212-1220, which is incorporated herein by reference in its entirety.

Optic neuritis is a demyelinating inflammation of the optic nerve and it typically affects young adults ranging from 18 to 45 years of age. Patients usually present with an acute reduction of visual acuity, orbital pain exacerbated by eye movements, dyschromatopsia, and an afferent papillary defect, with or without swelling of the optic nerve head. There is a strong association between optic neuritis and multiple sclerosis (MS), an acute inflammatory demyelinating disease of the central nervous system (CNS), in which optic neuritis is the initial presentation of MS for approximately 20% of MS patients and a risk of developing MS by 15 years after the onset of optic neuritis is 50%. In one embodiment, the ocular condition treated using the rAAV vectors described herein, is optic neuritis. See, Kimura et al, cited above. In another embodiment, the ocular condition is autoimmune encephalomyelitis (EAE). In another embodiment, the condition is MS.

The results shown herein indicate that delivery of the proviral plasmid or of a recombinant adeno-associated virus (AAV) containing the NRF2 cDNA to eyes of mice leads to expression of NRF2 and in animals with optic neuritis, can ameliorate the disorder. Exemplified herein are AAV2 based vectors, but in other embodiments, AAV serotypes that transduce retinal ganglion cells efficiently after intravitreal injection (such as AAV7m8 and AAV8 bp, both described herein) are also useful herein.

This invention describes reagents that are useful, in one embodiment, to prevent loss of axons/myelin in the optic nerve when it faces inflammatory, autoimmune, inherited or acquired disease. The reagents are delivered directly to retinal ganglion cells, whose axons comprise the optic nerve. The delivery (intravitreal injection) can be administered as an in office treatment. The effect of the reagents to be delivered (NRF2 and SIRT) is amplified because these reagents control the function of multiple survival pathways. Both proteins are normally produced in the human retina and so will not be viewed as foreign proteins (and engender an immune response). These reagents, delivered by rAAV, can both prevent acute loss of retinal ganglion cells and also prevent their loss going forward as the disease waxes and wanes.

In one aspect, the methods and compositions described herein involve compositions and methods for delivering a nucleic acid sequence encoding nuclear factor erythroid-derived 2, like 2 (NRF2, also known as NFE2L2) protein to subjects in need thereof for the treatment of optic neuropathy. In one embodiment, such compositions involve codon optimization of the NRF2 coding sequence. Such compositions include, in one embodiment, the hNRF2 coding sequence shown in SEQ ID NO: 7. It is desirable to increase the efficacy of the product, and thus, increase safety, since a lower dose of reagent may be used. Also encompassed herein are compositions which include the native NRF2 coding sequences, as shown in SEQ ID NO: 4.

In another aspect, the methods and compositions described herein involve compositions and methods for delivering a nucleic acid sequence encoding SIRT1 protein to subjects in need thereof for the treatment of optic neuropathy. In one embodiment, such compositions involve codon optimization of the SIRT1 coding sequence. It is desirable to increase the efficacy of the product, and thus, increase safety, since a lower dose of reagent may be used. In one embodiment, the coding sequence is that shown in SEQ ID NO: 12. Also encompassed herein are compositions which include the native SIRT1 coding sequences, as shown in SEQ ID NO: 2.

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

The term “percent (%) identity.”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity.”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available [e.g., BLAST, ExPASy: ClustalO: FASTA: using, e.g., Needleman-Wunsch algorithm, Smith-Waterman algorithm]. Alignments are performed using any of a variety of publicly or commercially available Multiple Sequence Alignment Programs. Sequence alignment programs are available for amino acid sequences, e.g., the “Clustal Omega” “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13): 2682-2690 (1999).

Multiple sequence alignment programs are also available for nucleic acid sequences. Examples of such programs include, “Clustal Omega” “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

In one aspect, a codon optimized, engineered nucleic acid sequence encoding human NRF2 is provided. Preferably, the codon optimized NRF2 coding sequence has less than about 80% identity, preferably about 75% identity or less to the full-length native NRF2 coding sequence (SEQ ID NO: 4). In one embodiment, the codon optimized NRF2 coding sequence has about 74% identity with the native NRF2 coding sequence of SEQ ID NO: 4. In one embodiment, the codon optimized NRF2 coding sequence is characterized by improved translation rate as compared to native NRF2 following AAV-mediated delivery (e.g., rAAV). In one embodiment, the codon optimized NRF2 coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the full length native NRF2 coding sequence of SEQ ID NO: 4. In one embodiment, the codon optimized nucleic acid sequence is a variant of SEQ ID NO: 7. In another embodiment, the codon optimized nucleic acid sequence a sequence sharing about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or greater identity with SEQ ID NO: 7. In one embodiment, the codon optimized nucleic acid sequence is SEQ ID NO: 7. In another embodiment, the nucleic acid sequence is codon optimized for expression in humans. In other embodiments, a different NRF2 coding sequence is selected.

In one aspect, a codon optimized, engineered nucleic acid sequence encoding human SIRT1 is provided. Preferably, the codon optimized SIRT1 coding sequence has less than about 80% identity, preferably about 75% identity or less to the full-length native SIRT1 coding sequence (SEQ ID NO: 2). In one embodiment, the codon optimized SIRT1 coding sequence has about 73% identity with the native SIRT1 coding sequence of SEQ ID NO: 2. In one embodiment, the codon optimized SIRT1 coding sequence is characterized by improved translation rate as compared to native SIRT1 following AAV-mediated delivery (e.g., rAAV). In one embodiment, the codon optimized SIRT1 coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the full length native SIRT1 coding sequence of SEQ ID NO: 2. In another embodiment, the nucleic acid sequence is codon optimized for expression in humans. In one embodiment, the nucleic acid sequence is the sequence of SEQ ID NO: 12. In other embodiments, a different SIRT1 coding sequence is selected.

Codon-optimized coding regions can be designed by various different methods. This optimization may be performed using methods which are available on-line (e.g., GeneArt), published methods, or a company which provides codon optimizing services, e.g., DNA2.0 (Menlo Park, CA). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

By “engineered” is meant that the nucleic acid sequences encoding the SIRT1 or NRF2 protein described herein are assembled and placed into any suitable genetic element, e.g., naked DNA, phage, transposon, cosmid, episome, etc., which transfers the NRF2 or SIRT1 sequences carried thereon to a host cell, e.g., for generating non-viral delivery systems (e.g., RNA-based systems, naked DNA, or the like) or for generating viral vectors in a packaging host cell and/or for delivery to a host cells in a subject. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

As used herein, the term “host cell” may refer to the packaging cell line in which a recombinant AAV is produced from a production plasmid. In the alternative, the term “host cell” may refer to any target cell in which expression of the coding sequence is desired. Thus, a “host cell,” refers to a prokaryotic or eukaryotic cell that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to the cells employed to generate and package the viral vector or recombinant virus. In other embodiments herein, the term “host cell” refers to cultures of ocular cells or other neuronal cells of various mammalian species for in vitro assessment of the compositions described herein.

Still in other embodiments, the term “host cell” is intended to reference the target cells of the subject being treated in vivo for optic neuropathy. In one embodiment, the host cell or target cell is an ocular cell. As used herein, the term “ocular cells” refers to any cell in, or associated with the function of, the eye. The term may refer to any one of photoreceptor cells, including rod photoreceptors, cone photoreceptors and photosensitive ganglion cells, retinal pigment epithelium (RPE) cells, Mueller cells, choroidal cells, bipolar cells, horizontal cells, and amacrine cells. In one embodiment, the ocular cells are ganglion cells. In another embodiment, the host cell or target cell is a neuronal cell.

In one embodiment, the nucleic acid sequence encoding SIRT1 or NRF2 further comprises a nucleic acid encoding a tag polypeptide covalently linked thereto. The tag polypeptide may be selected from known “epitope tags” including, without limitation, a myc tag polypeptide, a glutathione-S-transferase tag polypeptide, a green fluorescent protein tag polypeptide, a myc-pyruvate kinase tag polypeptide, a His6 tag polypeptide, an influenza virus hemagglutinin tag polypeptide, a flag tag polypeptide, and a maltose binding protein tag polypeptide. Exemplified herein is the use of a FLAG tag polypeptide. Another example is an HA tag. In one embodiment, the HA tag has a coding sequence reproduced as nt 4222 to nt 4248 of SEQ ID NO: 28.

In another aspect, an expression cassette comprising a nucleic acid sequence that encodes SIRT1 or NRF2 is provided. In one embodiment, the sequence is a codon optimized sequence. In another embodiment, the codon optimized nucleic acid sequence is SEQ ID NO: 7 encoding human NRF2. In another embodiment, the nucleic acid sequence is SEQ ID NO: 4 encoding human NRF2. In another embodiment, the nucleic acid sequence is SEQ ID NO: 2 encoding human SIRT. In another embodiment, the nucleic acid sequence is SEQ ID NO: 12 encoding human SIRT.

As used herein, an “expression cassette” refers to a nucleic acid molecule which comprises the coding sequences for SIRT1 or NRF2 protein, promoter, and may include other regulatory sequences therefor, which cassette may be packaged into the capsid of a viral vector (e.g., a viral particle). Typically, such an expression cassette for generating a viral vector contains the SIRT1 or NRF2 sequences described herein flanked by packaging signals of the viral genome and other expression control sequences such as those described herein. For example, for an AAV viral vector, the packaging signals are the 5′ inverted terminal repeat (ITR) and the 3′ ITR. When packaged into the AAV capsid, the ITRs in conjunction with the expression cassette may be referred to herein as the “recombinant AAV (rAAV) genome” or “vector genome”. In one embodiment, an expression cassette comprises a codon optimized nucleic acid sequence that encodes SIRT1 or NRF2 protein. In one embodiment, the cassette provides the codon optimized SIRT1 or NRF2 operatively associated with expression control sequences that direct expression of the codon optimized nucleic acid sequence that encodes SIRT1 or NRF2 in a host cell.

In another embodiment, an expression cassette for use in an AAV vector is provided. In that embodiment, the AAV expression cassette includes at least one AAV inverted terminal repeat (ITR) sequence. In another embodiment, the expression cassette comprises 5′ITR sequences and 3′ ITR sequences. In one embodiment, the 5′ and 3′ ITRs flank the nucleic acid sequence that encodes SIRT, optionally with additional sequences which direct expression of the nucleic acid sequence that encodes SIRT1 in a host cell. In one embodiment, the 5′ and 3′ ITRs flank the nucleic acid sequence that encodes NRF2, optionally with additional sequences which direct expression of the nucleic acid sequence that encodes NRF2 in a host cell. Thus, as described herein, a AAV expression cassette is meant to describe an expression cassette as described above flanked on its 5′ end by a 5′AAV inverted terminal repeat sequence (ITR) and on its 3′ end by a 3′ AAV ITR. Thus, this rAAV genome contains the minimal sequences required to package the expression cassette into an AAV viral particle, i.e., the AAV 5′ and 3′ ITRs. The AAV ITRs may be obtained from the ITR sequences of any AAV, such as described herein. These ITRs may be of the same AAV origin as the capsid employed in the resulting recombinant AAV, or of a different AAV origin (to produce an AAV pseudotype). In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, the AAV vector genome comprises an AAV 5′ ITR, the NRF2 or SIRT1 coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used. Each rAAV genome can be then introduced into a production plasmid.

As used herein, the term “regulatory sequences”, “transcriptional control sequence” or “expression control sequence” refers to DNA sequences, such as initiator sequences, enhancer sequences, and promoter sequences, which induce, repress, or otherwise control the transcription of protein encoding nucleic acid sequences to which they are operably linked.

As used herein, the term “operably linked” or “operatively associated” refers to both expression control sequences that are contiguous with the nucleic acid sequence encoding the NRF2 or SIRT1 and/or expression control sequences that act in trans or at a distance to control the transcription and expression thereof.

In one aspect, a vector comprising any of the expression cassettes described herein is provided. As described herein, such vectors can be plasmids of variety of origins and are useful in certain embodiments for the generation of recombinant replication defective viruses as described further herein.

A “vector” as used herein is a nucleic acid molecule into which an exogenous or heterologous or engineered nucleic acid transgene may be inserted which can then be introduced into an appropriate host cell. Vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and (primarily in yeast and bacteria) “artificial chromosomes.” Certain plasmids are described herein.

In one embodiment, the vector is a non-viral plasmid that comprises an expression cassette described thereof, e.g., “naked DNA”, “naked plasmid DNA”, RNA, and mRNA: coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. Sec, e.g., X. Su et al, Mol. Pharmaceutics, 2011, 8 (3), pp 774-787: web publication: Mar. 21, 2011: WO2013/182683, WO 2010/053572 and WO 2012/170930, all of which are incorporated herein by reference. Such non-viral NRF2 or SIRT1 vector may be administered by the routes described herein. The viral vectors, or non-viral vectors, can be formulated with a physiologically acceptable carrier for use in gene transfer and gene therapy applications.

In another embodiment, the vector is a viral vector that comprises an expression cassette described therein. “Virus vectors” are defined as replication defective viruses containing the exogenous or heterologous SIRT1 or NRF2 nucleic acid transgene. In one embodiment, an expression cassette as described herein may be engineered onto a plasmid which is used for drug delivery or for production of a viral vector. Suitable viral vectors are preferably replication defective and selected from amongst those which target ocular cells. Viral vectors may include any virus suitable for gene therapy, including but not limited to adenovirus: herpes virus; lentivirus: retrovirus: parvovirus, etc. However, for ease of understanding, the adeno-associated virus is referenced herein as an exemplary virus vector.

A “replication-defective virus” or “viral vector” refers to a synthetic or recombinant viral particle in which an expression cassette containing a gene of interest is packaged in a viral capsid or envelope, where any viral genomic sequences also packaged within the viral capsid or envelope are replication-deficient: i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In another embodiment, a recombinant adeno-associated virus (rAAV) vector is provided. The rAAV compromises an AAV capsid, and a vector genome packaged therein. The vector genome comprises, in one embodiment: (a) an AAV 5′ inverted terminal repeat (ITR) sequence; (b) a promoter: (c) a coding sequence encoding a human SIRT; and (d) an AAV 3′ ITR. In another embodiment, the vector genome is the expression cassette described herein. In one embodiment, the SIRT1 sequence encodes a full length protein. In one embodiment, the SIRT1 sequence is the protein sequence of SEQ ID NO: 1. In another embodiment, the coding sequence is SEQ ID NO: 2 or a variant thereof. In another embodiment, the coding sequence is SEQ ID NO: 12 or a variant thereof.

The vector genome comprises, in another embodiment: (a) an AAV 5′ inverted terminal repeat (ITR) sequence: (b) a promoter: (c) a coding sequence encoding a human NRF2; and (d) an AAV 3′ ITR. In another embodiment, the vector genome is the expression cassette described herein. In one embodiment, the NRF2 sequence encodes a full length protein. In one embodiment, the NRF2 sequence is the protein sequence of SEQ ID NO: 3. In another embodiment, the coding sequence is SEQ ID NO: 4 or a variant thereof. In another embodiment, the coding sequence is SEQ ID NO: 7 or a variant thereof.

Adeno-associated virus (AAV), a member of the Parvovirus family, is a small nonenveloped, icosahedral virus with single-stranded linear DNA genomes of 4.7 kilobases (kb) to 6 kb. Among known AAV serotypes are AAV1, AAV2, AAV3, AAV4, AAV5, AAV6, AAV7, AAV8, AAV9 and others. The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, VA). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

Fragments of AAV may be readily utilized in a variety of vector systems and host cells. Among desirable AAV fragments are the cap proteins, including the vp1, vp2, vp3 and hypervariable regions, the rep proteins, including rep 78, rep 68, rep 52, and rep 40, and the sequences encoding these proteins. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a novel AAV sequence of the invention (e.g., a fragment of a vp1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

The term “AAV” or “AAV serotype” as used herein refers to the dozens of naturally occurring and available adeno-associated viruses, as well as artificial AAVs. Among the AAVs isolated or engineered from human or non-human primates (NHP) and well characterized, human AAV2 is the first AAV that was developed as a gene transfer vector: it has been widely used for efficient gene transfer experiments in different target tissues and animal models. Unless otherwise specified, the AAV capsid, ITRs, and other selected AAV components described herein, may be readily selected from among any AAV, including, without limitation, AAV1, AAV2, AAV3, AAV4, AAV5, AAV6, AAV7, AAV8, AAV9, AAV8 bp, AAV7M8 and AAVAnc80, variants of any of the known or mentioned AAVs or AAVs yet to be discovered or variants or mixtures thereof. See, e.g., WO 2005/033321, which is incorporated herein by reference. In one embodiment, the AAV capsid is an AAV2 capsid. In another embodiment, the AAV capsid is an AAV8 bp capsid. See, WO 2014/024282, which is incorporated herein by reference. In another embodiment, the AAV capsid is an AAV7m8 capsid, which has shown preferential delivery to the outer retina. See, Dalkara et al, In Vivo-Directed Evolution of a New Adeno-Associated Virus for Therapeutic Outer Retinal Gene Delivery from the Vitreous, Sci Transl Med 5, 189ra76 (2013), which is incorporated herein by reference. In another embodiment, the rAAV capsid is selected from an AAV8 capsid or variant thereof, an AAV6 capsid or variant thereof, an AAV9 capsid or variant thereof, an AAV7 capsid or variant thereof, an AAV5 capsid or variant thereof, an AAV2 capsid or variant thereof, an AAV1 capsid or variant thereof, an AAV3 capsid or variant thereof, and an AAV4 capsid or variant thereof.

In one embodiment, a recombinant adeno-associated virus (rAAV) vector is provided which comprises an AAV7m8 capsid and an expression cassette described herein, wherein said expression cassette comprises nucleic acid sequences encoding SIRT1, inverted terminal repeat sequences and expression control sequences that direct expression of SIRT1 in a host cell.

In one embodiment, a recombinant adeno-associated virus (rAAV) vector is provided which comprises an AAV7m8 capsid and an expression cassette described herein, wherein said expression cassette comprises nucleic acid sequences encoding NRF2, inverted terminal repeat sequences and expression control sequences that direct expression of NRF2 in a host cell.

In one embodiment, a recombinant adeno-associated virus (rAAV) vector is provided which comprises an AAV2 capsid and an expression cassette described herein, wherein said expression cassette comprises nucleic acid sequences encoding SIRT1, inverted terminal repeat sequences and expression control sequences that direct expression of SIRT1 in a host cell.

In one embodiment, a recombinant adeno-associated virus (rAAV) vector is provided which comprises an AAV2 capsid and an expression cassette described herein, wherein said expression cassette comprises nucleic acid sequences encoding NRF2, inverted terminal repeat sequences and expression control sequences that direct expression of NRF2 in a host cell.

In still a further embodiment, a recombinant adeno-associated virus (AAV) vector is provided for delivery of the SIRT1 or NRF2 constructs and optimized sequences described herein. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. AAVs may be selected as sources for capsids of AAV viral vectors as identified above. See. e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10) and (Dalkara D, Byrne L C, Klimczak R R, Visel M, Yin L, Merigan W H, et al. In vivo-directed evolution of a new adeno-associated virus for therapeutic outer retinal gene delivery from the vitreous. Sci Transl Med (2013) 5(189): 189ra76. doi: 10.1126/scitranslmed.3005708) (AAV7m8). Each of these documents is incorporated herein by reference. These documents also describe other AAV capsids which may be selected for generating AAV and are incorporated by reference. In some embodiments, an AAV cap for use in the viral vector can be generated by mutagenesis (i.e., by insertions, deletions, or substitutions) of one of the aforementioned AAV capsids or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps.

As used herein, relating to AAV, the term variant means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). In one embodiment, the AAV capsid shares at least 95% identity with the AAV7m8 over the vp1, vp2 or vp3. In one embodiment, the AAV capsid shares at least 95% identity with the AAV2 over the vp1, vp2 or vp3. In another embodiment, the capsid is an AAV8 capsid with Y447F, Y733F and T494V mutations (also called “AAV8(C&G+T494V)” and “rep2-cap8(Y447F+733F+T494V)”), as described by Kay et al. Targeting Photoreceptors via Intravitreal Delivery Using Novel, Capsid-Mutated AAV Vectors, PLOS One. 2013; 8(4): e62097. Published online 2013 Apr. 26, which is incorporated herein by reference.

In one embodiment, it is desirable to utilize an AAV capsid, which shows tropism for the desired target cell, e.g., ganglion or other ocular cells. In one embodiment, the AAV capsid is a tyrosine capsid-mutant in which certain surface exposed tyrosine residues are substituted with phenylalanine (F). Such AAV variants are described, e.g., in Mowat et al, Tyrosine capsid-mutant AAV vectors for gene delivery to the canine retina from a subretinal or intravitreal approach, Gene Therapy 21, 96-105 (January 2014), which is incorporated herein by reference. In one embodiment, the AAV capsid is chosen from those that effectively transduce neuronal cells. In one embodiment, the AAV capsid is selected from AAV1, AAV2, AAV7, AAV8, AAV9, AAVrh. 10, AAV5, AAVhu. 11, AAV8DJ, AAVhu.32, AAVhu.37, AAVpi.2, AAVrh.8, AAVhu.48R3 and variants thereof. See, Royo, et al, Brain Res, 2008 January, 1190:15-22; Petrosyan et al, Gene Therapy, 2014 December, 21(12): 991-1000; Holehonnur et al, BMC Neuroscience, 2014, 15:28; and Cearley et al, Mol Ther. 2008 October; 16(10): 1710-1718, each of which is incorporated herein by reference.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/7m8 and AAV2/8 bp are exemplary pseudotyped vectors.

In another embodiment, a self-complementary AAV is used. “Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g. a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

In still another embodiment, the expression cassette, including any of those described herein is employed to generate a recombinant AAV genome.

In one embodiment, the expression cassette described herein is engineered into a suitable genetic element (vector) useful for generating viral vectors and/or for delivery to a host cell, e.g., naked DNA, phage, transposon, cosmid, episome, etc., which transfers the SIRT1 or NRF2 sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

For packaging an expression cassette or rAAV genome or production plasmid into virions, the ITRs are the only AAV components required in cis in the same construct as the expression cassette. In one embodiment, the coding sequences for the replication (rep) and/or capsid (cap) are removed from the AAV genome and supplied in trans or by a packaging cell line in order to generate the AAV vector.

Methods for generating and isolating AAV viral vectors suitable for delivery to a subject are known in the art. See. e.g., U.S. Pat. Nos. 7,790,449; 7,282,199: WO 2003/042397: WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

In yet another system, the expression cassette flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally. e.g., Grieger & Samulski, 2005, “Adeno-associated virus as a gene therapy vector: Vector development, production and clinical applications,” Adv. Biochem. Engin/Biotechnol. 99: 119-145: Buning et al., 2008, “Recent developments in adeno-associated virus vector technology,” J. Gene Med. 10:717-733; and the references cited below, each of which is incorporated herein by reference in its entirety.

The methods used to construct any embodiment of this invention are known to those with skill in nucleic acid manipulation and include genetic engineering, recombinant engineering, and synthetic techniques. See. e.g., Green and Sambrook et al, Molecular Cloning: A Laboratory Manual, Cold Spring Harbor Press, Cold Spring Harbor, NY (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

“Plasmids” generally are designated herein by a lower case p preceded and/or followed by capital letters and/or numbers, in accordance with standard naming conventions that are familiar to those of skill in the art. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

In one embodiment, the production plasmid is that described herein, or as described in WO2012/158757, which is incorporated herein by reference. Various plasmids are known in the art for use in producing rAAV vectors, and are useful herein. The production plasmids are cultured in the host cells which express the AAV cap and/or rep proteins. In the host cells, each rAAV genome is rescued and packaged into the capsid protein or envelope protein to form an infectious viral particle.

In one aspect, a production plasmid comprising an expression cassette comprising hNRF2 described above is provided. In one embodiment, the production plasmid is that shown in SEQ ID NO: 5, and FIG. 1. In another embodiment, the production plasmid is that shown in SEQ ID NO: 8. In another embodiment, the production plasmid is that shown in SEQ ID NO: 10. In another embodiment, the production plasmid is that shown in SEQ ID NO: 21. In another embodiment, the production plasmid is that shown in SEQ ID NO: 22. In another embodiment, the production plasmid is that shown in SEQ ID NO: 23. In another embodiment, the production plasmid is that shown in SEQ ID NO: 24. In another embodiment, the production plasmid is that shown in SEQ ID NO: 25. In another embodiment, the production plasmid is that shown in SEQ ID NO: 26. In another embodiment, the production plasmid is that shown in SEQ ID NO: 27. In another embodiment, the production plasmid is that shown in SEQ ID NO: 28. These plasmids are used in the examples for generation of the rAAV-human NRF2 vectors. In another aspect, a production plasmid comprising an expression cassette comprising hSIRT1 described above is provided. In one embodiment, the production plasmid is that shown in SEQ ID NO: 6, and FIG. 2. In another embodiment, the production plasmid is that shown in SEQ ID NO: 9. In another embodiment, the production plasmid is that shown in SEQ ID NO: 13. In another embodiment, the production plasmid is that shown in SEQ ID NO: 14. In another embodiment, the production plasmid is that shown in SEQ ID NO: 15. In another embodiment, the production plasmid is that shown in SEQ ID NO: 16. In another embodiment, the production plasmid is that shown in SEQ ID NO: 17. In another embodiment, the production plasmid is that shown in SEQ ID NO: 18. In another embodiment, the production plasmid is that shown in SEQ ID NO: 19. In another embodiment, the production plasmid is that shown in SEQ ID NO: 20. This plasmid is used in the examples for generation of the rAAV-human SIRT1 vector. Such a plasmid is one that contains a 5′ AAV ITR sequence: a selected promoter: a polyA sequence; and a 3′ ITR: additionally, it also contains a stuffer sequence, such as lambda.

In one embodiment, a non-coding lambda stuffer region is included in the vector backbone. An example of p643 which includes the hNRF2 coding sequence can be found in SEQ ID NO: 5. An example of p643 which includes the hSIRT1 coding sequence can be found in SEQ ID NO: 6. In another embodiment, the production plasmid is modified to optimized vector plasmid production efficiency. Such modifications include addition of other neutral sequences, or deletion of portion(s) of or the entire lambda stuffer sequence to modulate the level of supercoil of the vector plasmid. Such modifications are contemplated herein. In other embodiments, terminator and other sequences are included in the plasmid.

In one embodiment, the rAAV vector genome is of a size between about 3 kilobases (kb) to about 6 kb, about 4.7 kb to about 6 kb, about 3 kb to about 5.5 kb, or about 4.7 kb to 5.5 kb.

In certain embodiments, the rAAV expression cassette, the vector (such as rAAV vector), the virus (such as rAAV), or the production plasmid comprises AAV inverted terminal repeat sequences, a nucleic acid sequence that encodes NRF2, and expression control sequences that direct expression of the encoded protein in a host cell. In other embodiments, the rAAV expression cassette, the virus, the vector (such as rAAV vector), or the production plasmid further comprise one or more of an intron, a Kozak sequence, a polyA, post-transcriptional regulatory elements and others. In one embodiment, the post-transcriptional regulatory element is Woodchuck Hepatitis Virus (WHP) Posttranscriptional Regulatory Element (WPRE).

In certain embodiments, the rAAV expression cassette, the vector (such as rAAV vector), the virus (such as rAAV), or the production plasmid comprises AAV inverted terminal repeat sequences, a nucleic acid sequence that encodes SIRT1, and expression control sequences that direct expression of the encoded protein in a host cell. In other embodiments, the rAAV expression cassette, the virus, the vector (such as rAAV vector), or the production plasmid further comprise one or more of an intron, a Kozak sequence, a polyA, post-transcriptional regulatory elements and others. In one embodiment, the post-transcriptional regulatory element is Woodchuck Hepatitis Virus (WHP) Posttranscriptional Regulatory Element (WPRE).

The expression cassettes, vectors and plasmids include other components that can be optimized for a specific species using techniques known in the art including. e.g. codon optimization, as described herein. The components of the cassettes, vectors, plasmids and viruses or other compositions described herein include a promoter sequence as part of the expression control sequences. In another embodiment, the promoter is cell-specific. The term “cell-specific” means that the particular promoter selected for the recombinant vector can direct expression of the NRF2 or SIRT1 coding sequence in a particular cell type. In one embodiment, the promoter is specific for expression of the transgene in ocular cells. In one embodiment, the promoter is specific for expression of the transgene in ganglion cells. In one embodiment, the promoter is specific for expression of the transgene in photoreceptor cells. In another embodiment, the promoter is specific for expression in the rods and cones. In another embodiment, the promoter is specific for expression in the rods. In another embodiment, the promoter is specific for expression in the cones. In one embodiment, the photoreceptor-specific promoter is a human rhodopsin kinase promoter. The rhodopsin kinase promoter has been shown to be active in both rods and cones. See, e.g., Sun et al, Gene Therapy with a Promoter Targeting Both Rods and Cones Rescues Retinal Degeneration Caused by AIPL1 Mutations, Gene Ther. 2010 January; 17(1): 117-131, which is incorporated herein by reference in its entirety. In one embodiment, the promoter is modified to add one or more restriction sites to facilitate cloning.

In another embodiment, a neuron-specific promoter is used [see. e.g., the Lockery Lab neuron-specific promoters database, accessed at chinook.uoregon.edu/promoters.html]. Such neuron-specific promoters include, without limitation, e.g., synapsin I (SYN), calcium/calmodulin-dependent protein kinase II, tubulin alpha I, neuron-specific enolase and platelet-derived growth factor beta chain promoters. See, Hioki et al, Gene Therapy, June 2007, 14(11): 872-82, which is incorporated herein by reference. Other neuron-specific promoters include the 67 kDa glutamic acid decarboxylase (GAD67), homeobox Dlx5/6, glutamate receptor 1 (GluR1), preprotachykinin 1 (Tac 1) promoter, neuron-specific enolase (NSE) and dopaminergic receptor 1 (Drd1a) promoters. See, e.g., Delzor et al, Human Gene Therapy Methods. August 2012, 23(4): 242-254. In another embodiment, the promoter is a GUSb promoter.

In another embodiment, the promoter is a human rhodopsin promoter. In one embodiment, the promoter is modified to include restriction on the ends for cloning. See. e.g. Nathans and Hogness, Isolation and nucleotide sequence of the gene encoding human rhodopsin, PNAS, 81:4851-5 (August 1984), which is incorporated herein by reference in its entirety. In another embodiment, the promoter is a portion or fragment of the human rhodopsin promoter. In another embodiment, the promoter is a variant of the human rhodopsin promoter.

Other exemplary promoters include the human G-protein-coupled receptor protein kinase 1 (GRK1) promoter (Genbank Accession number AY327580). In another embodiment, the promoter is a 292 nt fragment (positions 1793-2087) of the GRK1 promoter (See, Beltran et al, Gene Therapy 2010 17:1162-74, which is hereby incorporated by reference in its entirety). In another preferred embodiment, the promoter is the human interphotoreceptor retinoid-binding protein proximal (IRBP) promoter. In one embodiment, the promoter is a 235 nt fragment of the hIRBP promoter. In one embodiment, the promoter is the RPGR proximal promoter (Shu et al. IOVS, May 2102, which is incorporated by reference in its entirety). Other promoters useful in the invention include, without limitation, the rod opsin promoter, the red-green opsin promoter, the blue opsin promoter, the cGMP-β-phosphodiesterase promoter (Qgueta et al, IOVS, Invest Ophthalmol Vis Sci. 2000 December; 41(13):4059-63), the mouse opsin promoter (Beltran et al 2010 cited above), the rhodopsin promoter (Mussolino et al, Gene Ther, July 2011, 18(7):637-45): the alpha-subunit of cone transducin (Morrissey et al, BMC Dev, Biol, January 2011, 11:3); beta phosphodiesterase (PDE) promoter: the retinitis pigmentosa (RP1) promoter (Nicord et al, J. Gene Med. December 2007, 9(12): 1015-23): the NXNL2/NXNL1 promoter (Lambard et al, PLOS One, October 2010, 5(10):e13025), the RPE65 promoter: the retinal degeneration slow/peripherin 2 (Rds/perph2) promoter (Cai et al, Exp Eye Res. 2010 August; 91(2): 186-94); and the VMD2 promoter (Kachi et al, Human Gene Therapy, 2009 (20:31-9)). Each of these documents is incorporated by reference herein in its entirety. In another embodiment, the promoter is selected from human human EF1α promoter, rhodopsin promoter, rhodopsin kinase, interphotoreceptor binding protein (IRBP), cone opsin promoters (red-green, blue), cone opsin upstream sequences containing the red-green cone locus control region, cone transducing, and transcription factor promoters (neural retina leucine zipper (Nr1) and photoreceptor-specific nuclear receptor Nr2e3, bZIP).

In another embodiment, the promoter is a ubiquitous or constitutive promoter. An example of a suitable promoter is a hybrid chicken β-actin (CBA) promoter with cytomegalovirus (CMV) enhancer elements, such as the sequence shown in nt 1443-3104 of SEQ ID NO: 5. In another embodiment, the promoter is the CB7 promoter. Other suitable promoters include the human β-actin promoter, the human elongation factor-1α promoter, the cytomegalovirus (CMV) promoter, the simian virus 40 promoter, and the herpes simplex virus thymidine kinase promoter. See, e.g., Damdindorj et al. (August 2014) A Comparative Analysis of Constitutive Promoters Located in Adeno-Associated Viral Vectors. PLOS ONE 9(8): e106472. Still other suitable promoters include viral promoters, constitutive promoters, regulatable promoters [see. e.g., WO 2011/126808 and WO 2013/04943]. Alternatively a promoter responsive to physiologic cues may be utilized in the expression cassette, rAAV genomes, vectors, plasmids and viruses described herein. Other promoters may be selected by one of skill in the art.

In a further embodiment, the promoter is selected from SV40 promoter, the dihydrofolate reductase promoter, and the phosphoglycerol kinase (PGK) promoter, rhodopsin kinase promoter, the rod opsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In another embodiment, the promoter is an inducible promoter. The inducible promoter may be selected from known promoters including the rapamycin/rapalog promoter, the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24:5:17105 and Daber R, Lewis M., A novel molecular switch. J Mol Biol. 2009 Aug. 28:391(4):661-70, Epub 2009 Jun. 21 which are both incorporated herein by reference in their entirety.

In a further embodiment, the promoter is a chicken beta-actin promoter with a nucleic acid sequence from nt 1443-3104 of SEQ ID NO: 5.

In one embodiment, the promoter is a human gamma-synuclein gene (i.e., human gamma-synuclein, human SNCG, hSNCG) promoter, such as the sequence shown in nt 1433 to nt 2362 of SEQ ID NO: 28. Also see, e.g., Chaffiol A et al. A New Promoter Allows Optogenetic Vision Restoration with Enhanced Sensitivity in Macaque Retina. Mol Ther. 2017 Nov. 1:25(11):2546-2560. doi: 10.1016/j.ymthe.2017.07.011. Epub 2017 Jul. 20.

In other embodiments, the expression cassette, vector, plasmid and virus described herein contain other appropriate transcription initiation, termination, enhancer sequences, efficient RNA processing signals such as splicing and polyadenylation (polyA) signals: TATA sequences; sequences that stabilize cytoplasmic mRNA: sequences that enhance translation efficiency (i.e., Kozak consensus sequence): introns: sequences that enhance protein stability; and when desired, sequences that enhance secretion of the encoded product. The expression cassette or vector may contain none, one or more of any of the elements described herein.

Examples of suitable polyA sequences include, e.g., a synthetic polyA or from bovine growth hormone (bGH), human growth hormone (hGH), SV40, rabbit β-globin (RGB), or modified RGB (mRGB). In a further embodiment, the poly A has a nucleic acid sequence from nt 5039 to nt 5246 of SEQ ID NO: 5.

In one embodiment, the expression cassette, vector, plasmid and virus described herein comprise a post-transcriptional regulatory element. In one embodiment, the post-transcriptional regulatory element is Woodchuck Hepatitis Virus (WHP) Posttranscriptional Regulatory Element (WPRE). Examples of sequence encoding a suitable WPRE are shown in SEQ ID NOs: 18, 19, 20, 25, 26, 27, and 28, e.g., nt 4279 to nt 4820 of SEQ ID NO: 28.

Examples of suitable enhancers include, e.g., the CMV enhancer, the RSV enhancer, the alpha fetoprotein enhancer, the TTR minimal promoter/enhancer, LSP (TH-binding globulin promoter/alpha 1-microglobulin/bikunin enhancer), an APB enhancer, ABPS enhancer, an alpha mic/bik enhancer, TTR enhancer, cn34, ApoE amongst others.

In one embodiment, a Kozak sequence is included upstream of the hNRF2 or hSIRT1 coding sequence to enhance translation from the correct initiation codon. In one embodiment, the hNRF2 or hSIRT1 coding sequence is placed under the control of a hybrid chicken β actin (CBA) promoter. This promoter consists of the cytomegalovirus (CMV) immediate early enhancer, the proximal chicken β actin promoter, and optionally, the CBA exon 1 flanked by intron 1 sequences. In another embodiment, the intron is selected from CBA, human beta globin, IVS2, SV40, bGH, alpha-globulin, beta-globulin, collagen, ovalbumin, p53, or a fragment thereof.

In one embodiment, the expression cassette, the vector, the plasmid and the virus contain a 5′ ITR, chicken beta-actin (CBA) promoter, CMV enhancer, human NRF2 coding sequence, bGH poly A and 3′ ITR. In a further embodiment, the vector genome includes nt 1253 to 5425 of SEQ ID NO: 5. In yet a further embodiment, the 5′ ITR has a nucleic acid sequence from nt 1253 to nt 1382 of SEQ ID NO: 5 and the 3′ITR has a nucleic acid sequence from nt 5296 to nt 5425 of SEQ ID NO: 5. In a further embodiment, the production plasmid has a sequence of SEQ ID NO: 5, also shown in FIG. 1. In a further embodiment, the production plasmid has a sequence of SEQ ID NO: 8. In a further embodiment, the production plasmid has a sequence of SEQ ID NO: 10. In a further embodiment, the production plasmid has a sequence of SEQ ID NO: 21. In a further embodiment, the production plasmid has a sequence of SEQ ID NO: 22. In a further embodiment, the production plasmid has a sequence of SEQ ID NO: 23. In a further embodiment, the production plasmid has a sequence of SEQ ID NO: 24. In a further embodiment, the vector genome includes nt 1253 to 5351 of SEQ ID NO: 8. In a further embodiment, the vector genome includes nt 1253 to 5359 of SEQ ID NO: 10. In a further embodiment, the vector genome comprises nt 1253 to nt 5359 of SEQ ID NO: 21. In a further embodiment, the vector genome comprises nt 1253 to nt 5425 of SEQ ID NO: 22. In a further embodiment, the vector genome comprises nt 1253 to nt 5351 of SEQ ID NO: 23. In a further embodiment, the vector genome comprises nt 1253 to nt 5378 of SEQ ID NO: 24.

In one embodiment, the expression cassette, the vector, the plasmid and the virus contain a 5′ ITR, a hSNCG promoter, human NRF2 coding sequence, bGH poly A and 3′ ITR. In one embodiment, the expression cassette, the vector, the plasmid and the virus contain a 5′ ITR, a hSNCG promoter, human NRF2 coding sequence, a WPRE sequence, bGH poly A and 3′ ITR. In a further embodiment, the vector genome comprises nt 1253 to nt 5253 of SEQ ID NO: 27. In yet a further embodiment, the 5′ ITR has a nucleic acid sequence from nt 1253 to nt 1382 of SEQ ID NO: 27 and the 3′ITR has a nucleic acid sequence from nt 5124 to nt 5253 of SEQ ID NO: 27. In a further embodiment, the production plasmid has a sequence of SEQ ID NO: 25. In a further embodiment, the production plasmid has a sequence of SEQ ID NO: 26. In a further embodiment, the production plasmid has a sequence of SEQ ID NO: 27. In a further embodiment, the production plasmid has a sequence of SEQ ID NO: 28. In a further embodiment, the vector genome comprises nt 1253 to nt 5253 of SEQ ID NO: 25. In a further embodiment, the vector genome comprises nt 1253 to nt 5280 of SEQ ID NO: 26. In a further embodiment, the vector genome comprises nt 1253 to nt 5280 of SEQ ID NO: 28.

In one embodiment, the expression cassette, the vector, the plasmid and the virus contain a 5′ ITR, chicken beta-actin (CBA) promoter, CMV enhancer, human SIRT1 coding sequence, bGH poly A and 3′ ITR. In a further embodiment, the expression cassette includes nt 1253 to 5854 of SEQ ID NO: 6. In yet a further embodiment, the 5′ ITR has a nucleic acid sequence from nt 1253 to nt 1382 of SEQ ID NO: 6 and the 3′ITR has a nucleic acid sequence from nt 5725 to nt 5854 of SEQ ID NO: 6. In a further embodiment, the production plasmid has a sequence of SEQ ID NO: 6, also shown in FIG. 2. In a further embodiment, the production plasmid has a sequence of SEQ ID NO: 9. In a further embodiment, the production plasmid has a sequence of SEQ ID NO: 11. In a further embodiment, the production plasmid has a sequence of SEQ ID NO: 13. In a further embodiment, the production plasmid has a sequence of SEQ ID NO: 14. In a further embodiment, the production plasmid has a sequence of SEQ ID NO: 15. In a further embodiment, the production plasmid has a sequence of SEQ ID NO: 16. In a further embodiment, the production plasmid has a sequence of SEQ ID NO: 17. In a further embodiment, the expression cassette includes nt 1253 to 5788 of SEQ ID NO: 9. In a further embodiment, the expression cassette comprises nt 1253 to 5792 of SEQ ID NO: 11. In a further embodiment, the expression cassette comprises nt 1253 to nt 5777 of SEQ ID NO: 13. In a further embodiment, the expression cassette comprises nt 1253 to nt 5854 of SEQ ID NO: 14. In a further embodiment, the expression cassette comprises nt 1253 to nt 5792 of SEQ ID NO: 15. In a further embodiment, the expression cassette comprises nt 1253 to nt 5819 of SEQ ID NO: 16. In a further embodiment, the expression cassette comprises nt 1253 to nt 5777 of SEQ ID NO: 17.

In one embodiment, the expression cassette, the vector, the plasmid and the virus contain a 5′ ITR, a hSNCG promoter, human SIRT1 coding sequence, bGH poly A and 3′ ITR. In one embodiment, the expression cassette, the vector, the plasmid and the virus contain a 5′ ITR, a hSNCG promoter, human SIRT1 coding sequence, a WPRE sequence, bGH poly A and 3′ ITR. In a further embodiment, the expression cassette comprises nt 1253 to nt 5694 of SEQ ID NO: 19. In yet a further embodiment, the 5′ ITR has a nucleic acid sequence from nt 1253 to nt 1382 of SEQ ID NO: 19 and the 3′ITR has a nucleic acid sequence from nt 5565 to nt 5694 of SEQ ID NO: 19. In a further embodiment, the production plasmid has a sequence of SEQ ID NO: 19. In a further embodiment, the production plasmid has a sequence of SEQ ID NO: 18. In a further embodiment, the production plasmid has a sequence of SEQ ID NO: 20. In a further embodiment, the expression cassette comprises nt 1253 to nt 5706 of SEQ ID NO: 18. In a further embodiment, the expression cassette comprises nt 1253 to nt 5721 of SEQ ID NO: 20.

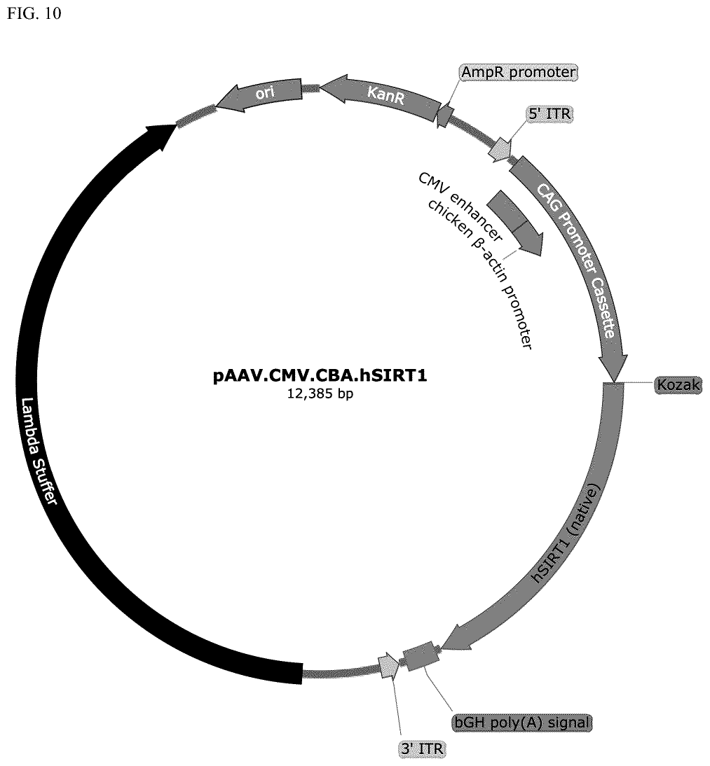

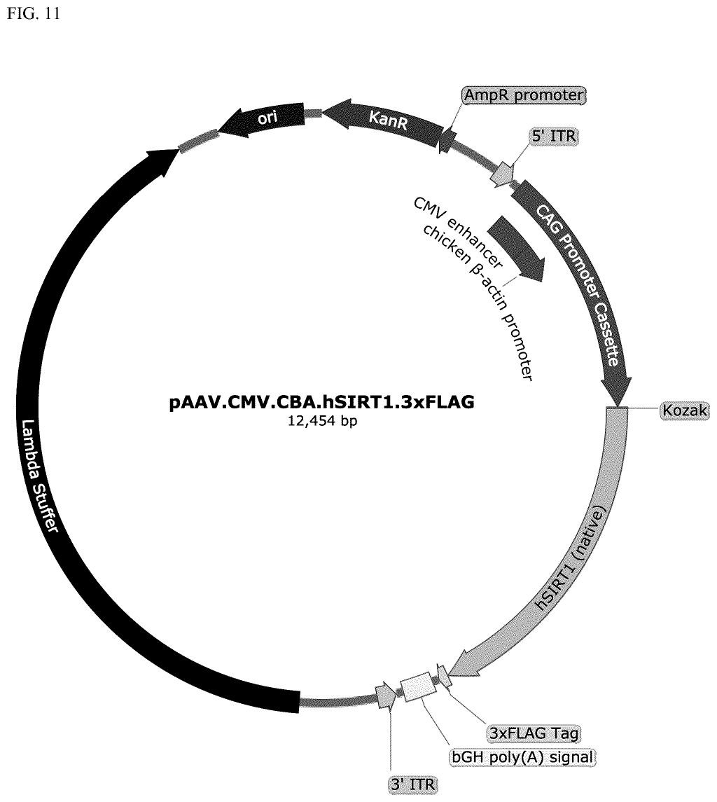

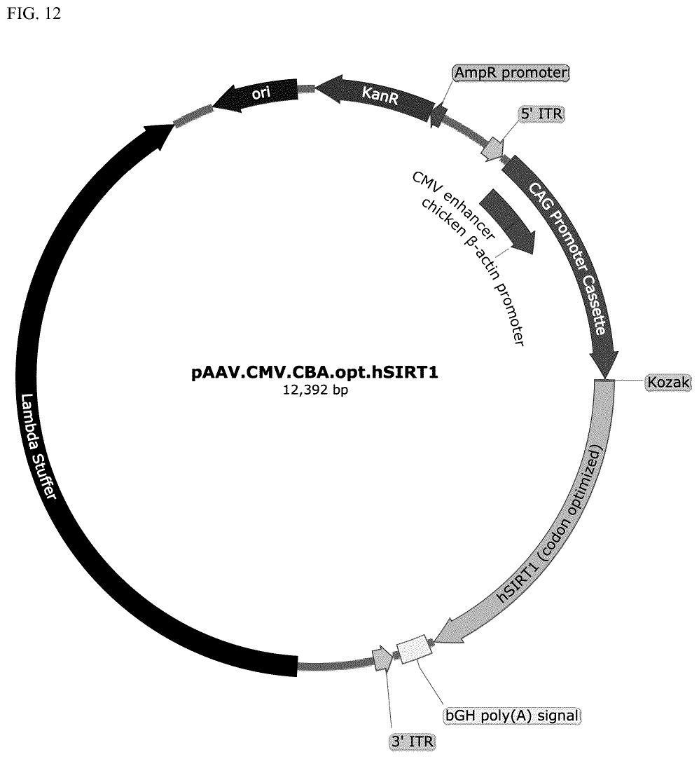

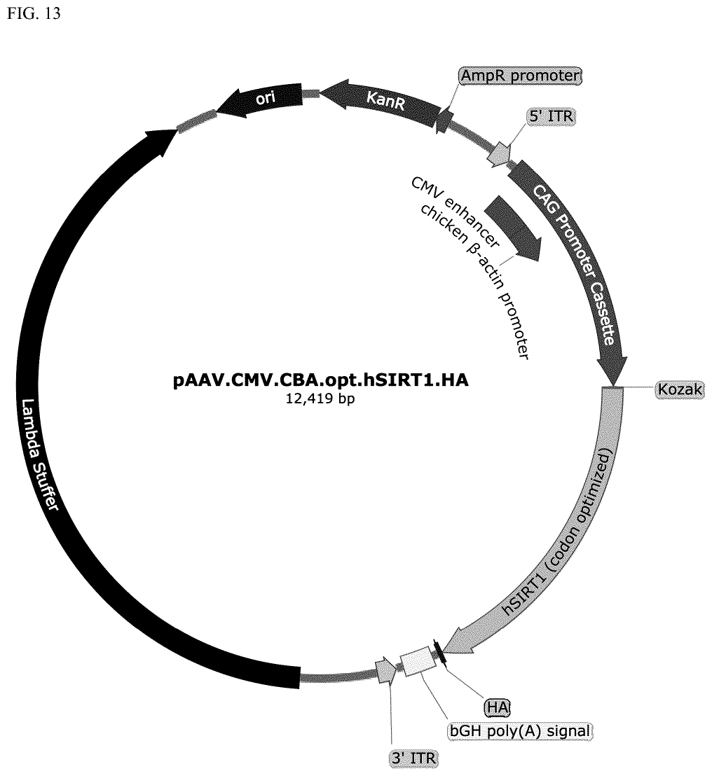

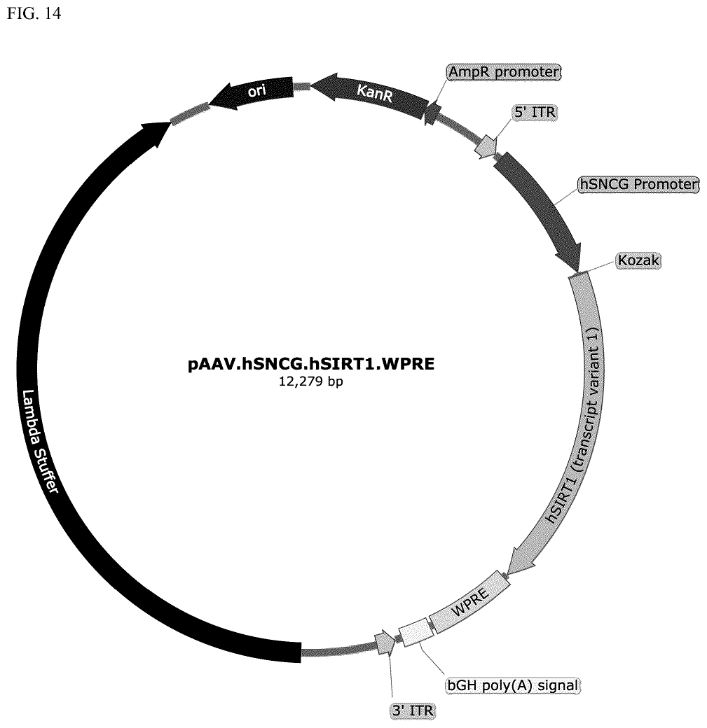

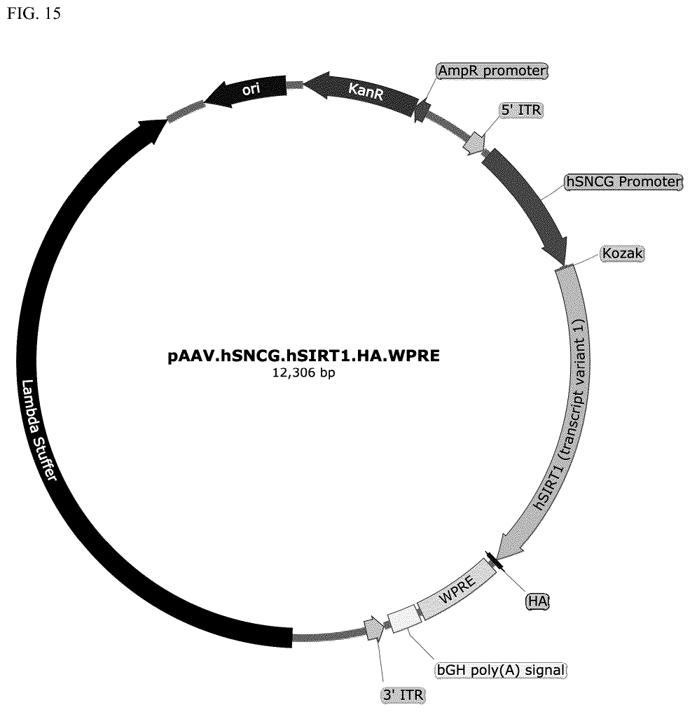

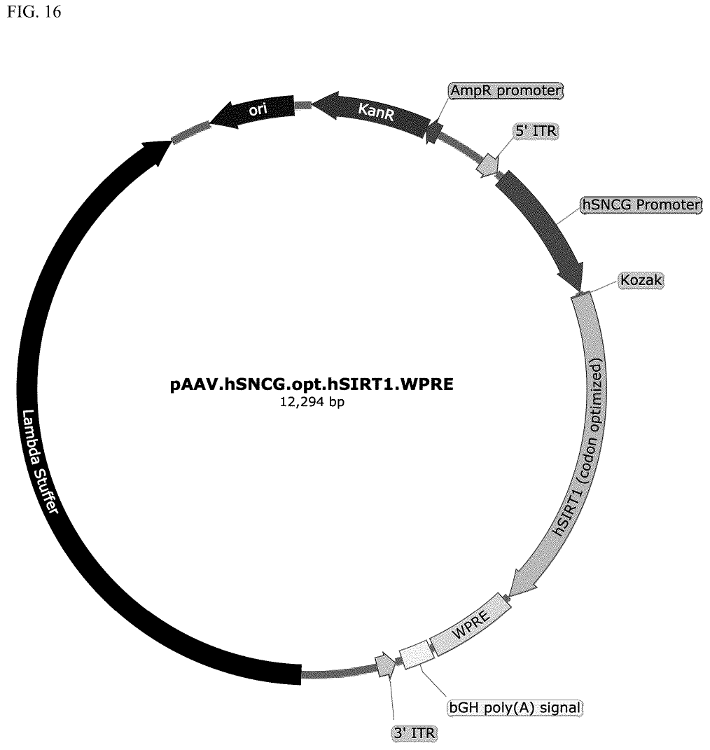

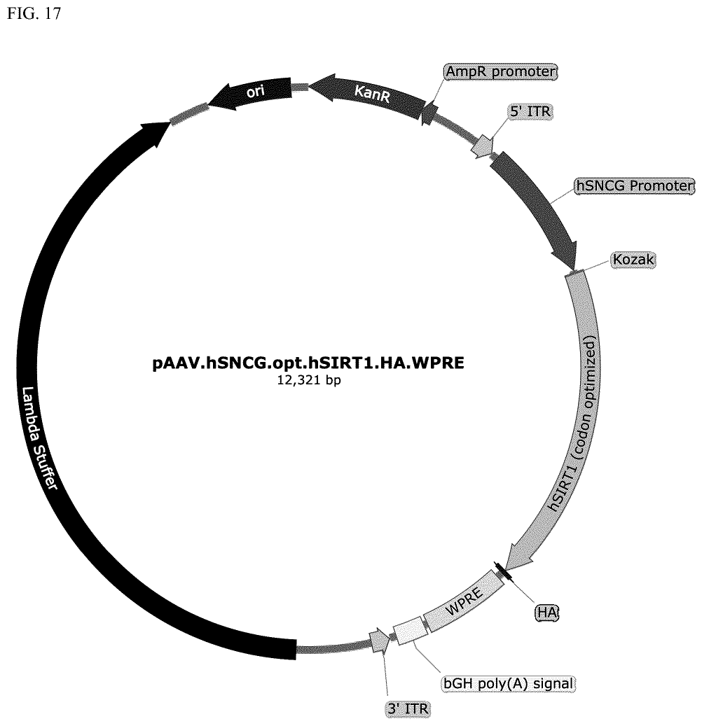

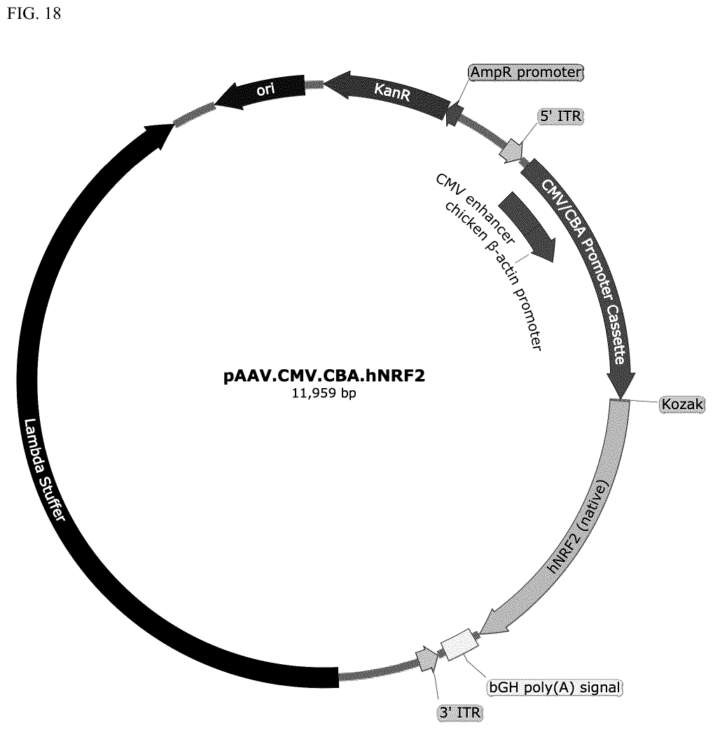

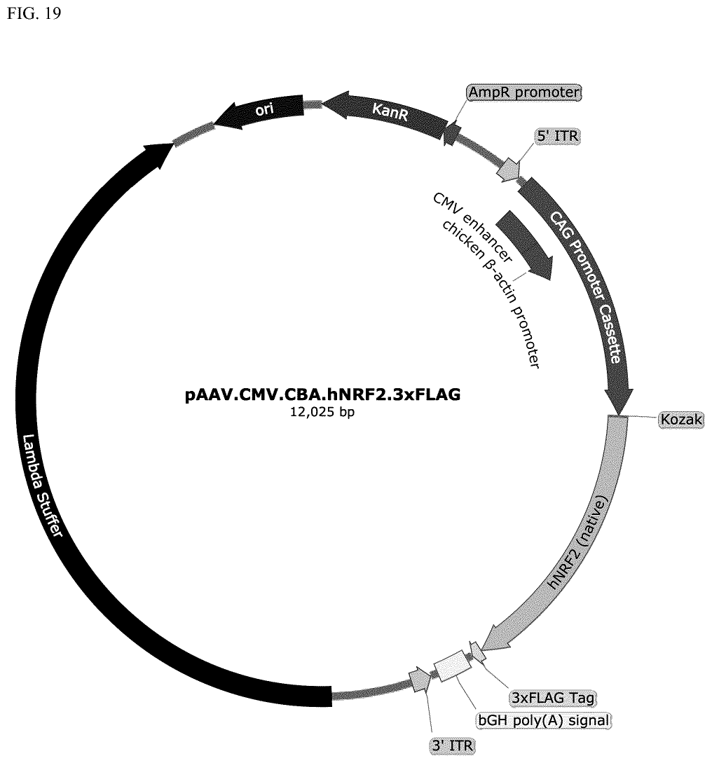

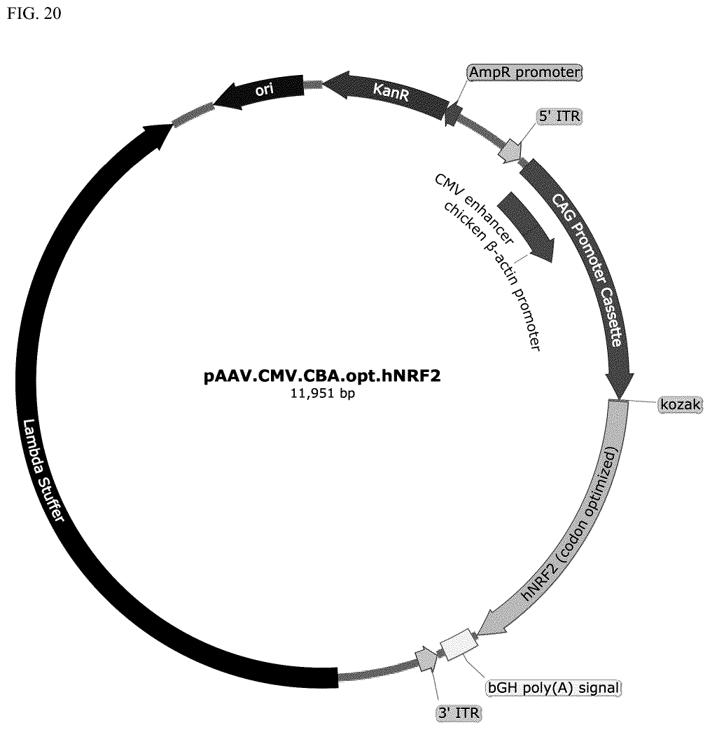

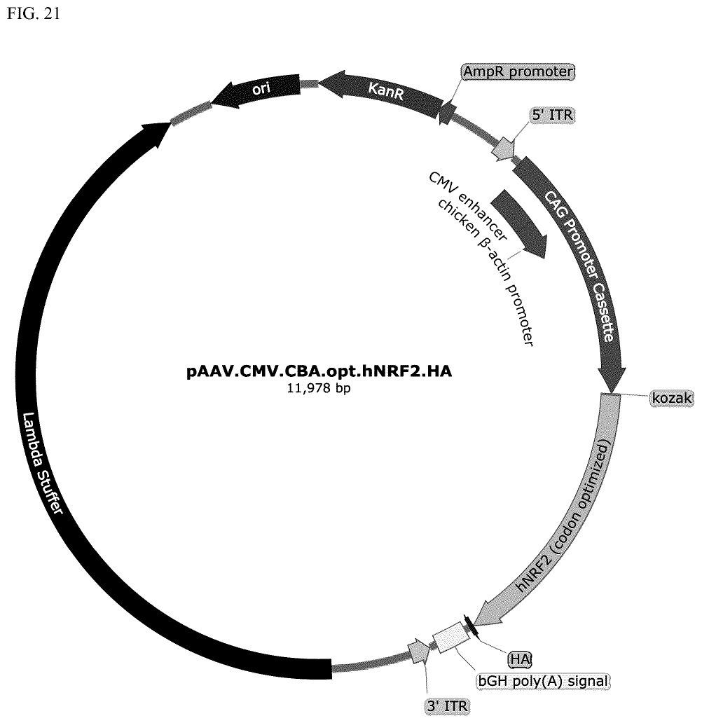

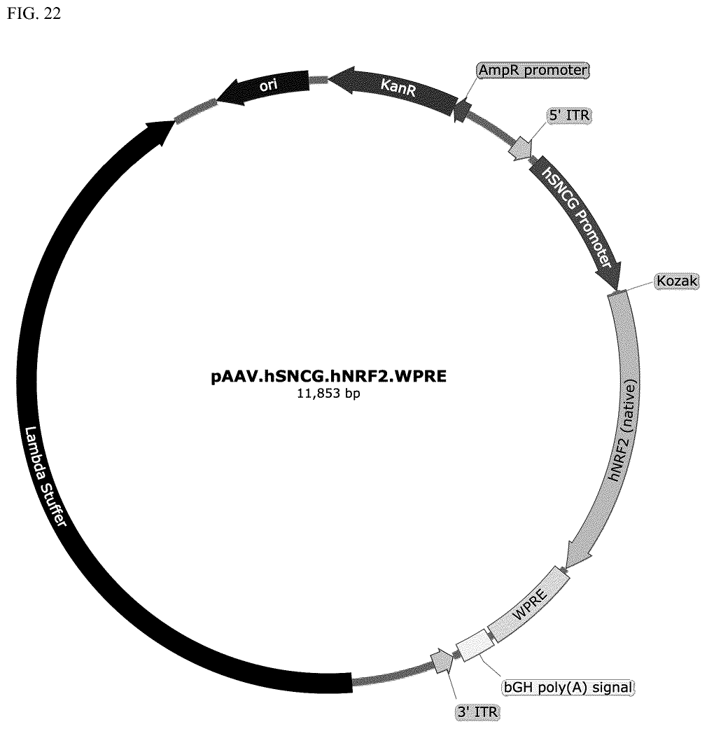

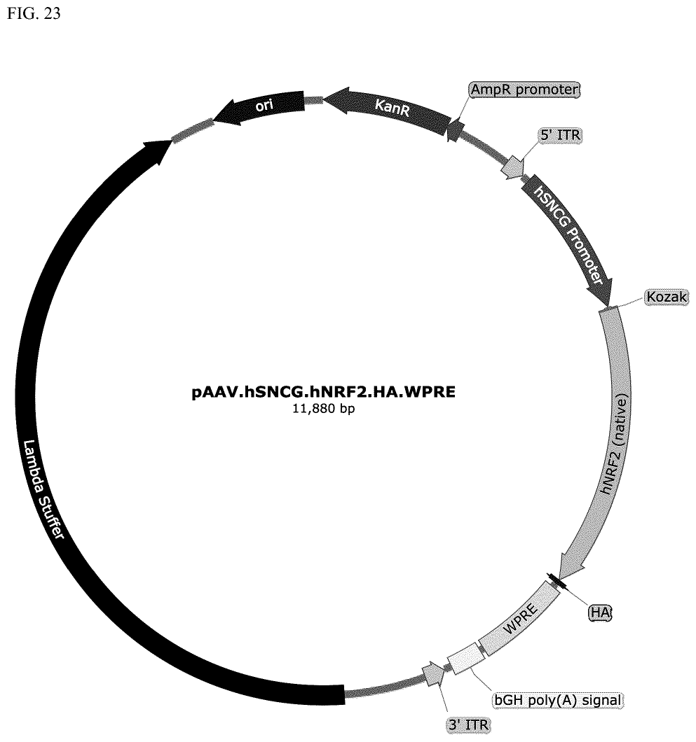

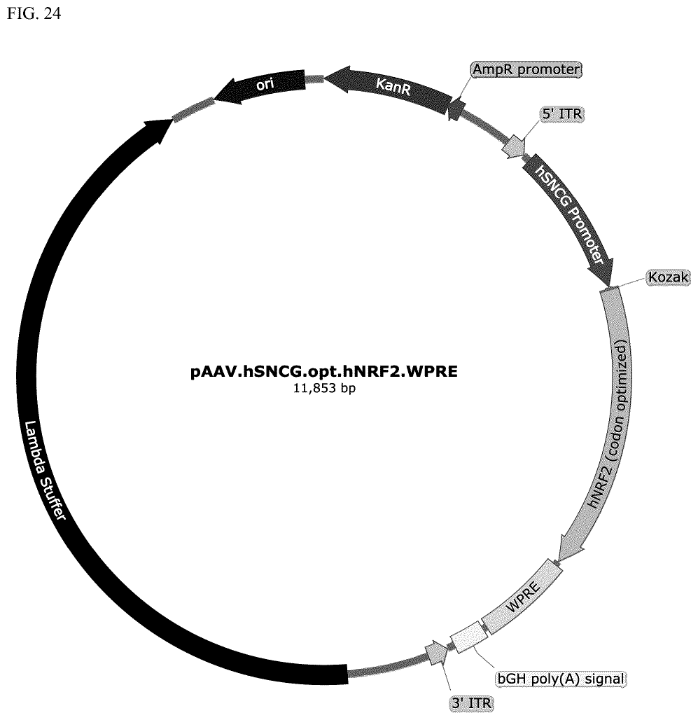

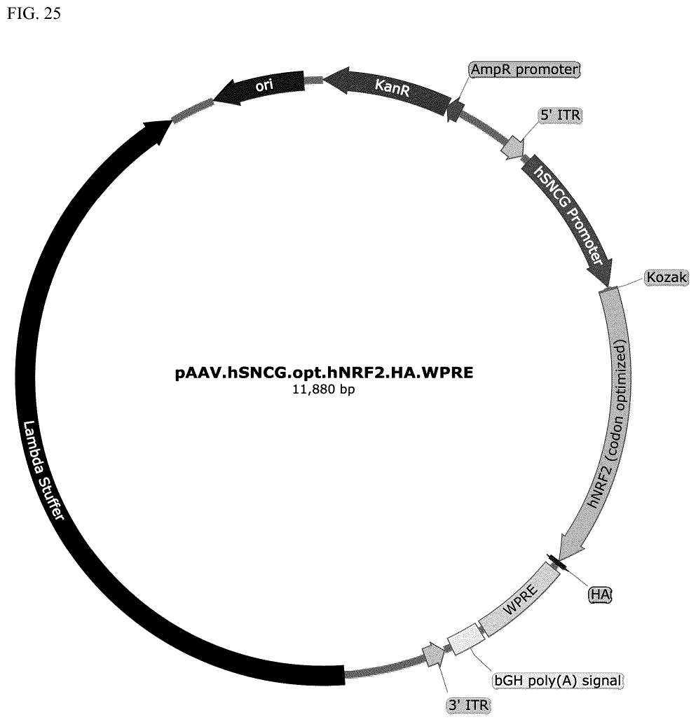

In certain embodiments, provided herein are the vector genome, the vector (such as rAAV vector), the virus (such as rAAV), or the production plasmid, as shown in FIGS. 1, 2, and 10 to 25.

In some embodiments, where a WPRE sequence is included in the vector genome, an alternate embodiment is contemplated wherein the WPRE is absent.

In another aspect, a method for treating ocular neuropathy and/or restoring visual function in a subject having in need thereof comprises delivering to a subject in need thereof a vector (such as rAAV) which encodes hNRF2, as described herein. In one embodiment, a method of treating a subject having ocular neuropathy with a rAAV described herein is provided.

In another aspect, a method for treating ocular neuropathy and/or restoring visual function in a subject having in need thereof comprises delivering to a subject in need thereof a vector (such as rAAV) which encodes hSIRT1, as described herein. In one embodiment, a method of treating a subject having ocular neuropathy with a rAAV described herein is provided.

In another aspect, a method of preserving retinal ganglion cell (RGC) function in a subject, comprising administering the an rAAV as described herein, is provided.

By “administering” as used in the methods means delivering the composition to the target cell. In one embodiment, the method involves delivering the composition by subretinal injection to the ganglion or other ocular cells. In another embodiment, intravitreal injection to the subject is employed. In another embodiment, subretinal injection to the subject is employed. In still another method, intravascular injections, such as injection via the palpebral vein may be employed. Still other methods of administration may be selected by one of skill in the art given this disclosure.

By “administering” or “route of administration” is meant delivery of a composition described herein, with or without a pharmaceutical carrier or excipient, to the subject. Routes of administration may be combined, if desired. In some embodiments, the administration is repeated periodically. The pharmaceutical compositions described herein are designed for delivery to subjects in need thereof by any suitable route or a combination of different routes. In some embodiments, direct delivery to the eye (optionally via ocular delivery, subretinal injection, intra-retinal injection, intravitreal, topical), or delivery via systemic routes is employed, e.g., intravascular, intraarterial, intraocular, intravenous, intramuscular, subcutaneous, intradermal, and other parental routes of administration. The nucleic acid molecules, the expression cassette and/or vectors described herein may be delivered in a single composition or multiple compositions. Optionally, two or more different AAV may be delivered, or multiple viruses [see, e.g., WO2011/126808 and WO 2013/049493]. In another embodiment, multiple viruses may contain different replication-defective viruses (e.g., AAV and adenovirus), alone or in combination with proteins.

Also provided herein are pharmaceutical compositions. The pharmaceutical compositions described herein are designed for delivery to subjects in need thereof by any suitable route or a combination of different routes. In some embodiments, these delivery means are designed to avoid direct systemic delivery of the suspension containing the AAV composition(s) described herein. Suitably, this may have the benefit of reducing dose as compared to systemic administration, reducing toxicity and/or reducing undesirable immune responses to the AAV and/or transgene product.

In yet other aspects, these nucleic acid sequences, vectors, expression cassettes and rAAV viral vectors are useful in a pharmaceutical composition, which also comprises a pharmaceutically acceptable carrier, excipient, buffer, diluent, surfactant, preservative and/or adjuvant, etc. Such pharmaceutical compositions are used to express the NRF2 or SIRT1 in the host cells through delivery by such recombinantly engineered AA Vs or artificial AAVs.

To prepare these pharmaceutical compositions containing the nucleic acid sequences, vectors, expression cassettes and rAAV viral vectors, the sequences or vectors or viral vector is preferably assessed for contamination by conventional methods and then formulated into a pharmaceutical composition suitable for administration to the eye. Such formulation involves the use of a pharmaceutically and/or physiologically acceptable vehicle or carrier, particularly one suitable for administration to the eye, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, surfactant, or excipient etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravitreal or subretinal delivery, a pH of 6.8 to about 7.2 may be desired. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate ·7H2O), potassium chloride, calcium chloride (e.g., calcium chloride ·2H2O), dibasic sodium phosphate, and mixtures thereof, in water. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intravitreal delivery. In one example, the composition is formulated for subretinal delivery.

In one exemplary specific embodiment, the composition of the carrier or excipient contains 180 mM NaCl, 10 mM NaPi, pH7.3 with 0.0001%-0.01% Pluronic F68 (PF68). The exact composition of the saline component of the buffer ranges from 160 mM to 180 mM NaCl. Optionally, a different pH buffer (potentially HEPES, sodium bicarbonate, TRIS) is used in place of the buffer specifically described. Still alternatively, a buffer containing 0.9% NaCl is useful.

In the case of AAV viral vectors, quantification of the genome copies (“GC”), vector genomes (“VG”), or virus particles may be used as the measure of the dose contained in the formulation or suspension. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). In another method the effective dose of a recombinant adeno-associated virus carrying a nucleic acid sequence encoding the NRF2 or SIRT1 coding sequence is measured as described in S. K. McLaughlin et al, 1988 J. Virol., 62:1963, which is incorporated by reference in its entirety.

As used herein, the term “dosage” can refer to the total dosage delivered to the subject in the course of treatment, or the amount delivered in a single unit (or multiple unit or split dosage) administration. The pharmaceutical virus compositions can be formulated in dosage units to contain an amount of replication-defective virus carrying the nucleic acid sequences encoding NRF2 or SIRT1 as described herein that is in the range of about 1.0×10⁹ GC to about 1.0×10¹⁵ GC per dose including all integers or fractional amounts within the range. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range. All dosages may be measured by any known method, including as measured by oqPCR or digital droplet PCR (ddPCR) as described in, e.g., M. Lock et al, Hum Gene Ther Methods. 2014 April; 25(2): 115-25. doi: 10.1089/hgtb.2013.131, which is incorporated herein by reference.

In one embodiment, an aqueous suspension suitable for administration to a patient with an ocular disorder is provided. The suspension comprises an aqueous suspending liquid and about 1×10⁹ GC or viral particles to about 1×10¹³ GC or viral particles per eye of a recombinant adeno-associated virus (rAAV) described herein useful as a therapeutic for an ocular disorder described herein. In one embodiment, the ocular disorder is optic neuritis.

It may also be desirable to administer multiple “booster” dosages of the pharmaceutical compositions of this invention. For example, depending upon the duration of the transgene within the ocular target cell, one may deliver booster dosages at 6 month intervals, or yearly following the first administration. The fact that AAV-neutralizing antibodies were not generated by administration of the rAAV vector should allow additional booster administrations.

Such booster dosages and the need therefor can be monitored by the attending physicians, using, for example, the retinal and visual function tests and the visual behavior tests described in the examples below. Other similar tests may be used to determine the status of the treated subject over time. Selection of the appropriate tests may be made by the attending physician. Still alternatively, the method of this invention may also involve injection of a larger volume of virus-containing solution in a single or multiple injection to allow levels of visual function close to those found in wildtype retinas.

In another embodiment, the amount of the vectors, the virus and the replication-defective virus described herein carrying the codon optimized nucleic acid sequences encoding SIRT1 or NRF2 are in the range of about 1.0×10⁷ VG per eye to about 1.0×10¹⁵ VG per eye including all integers or fractional amounts within the range. In one embodiment, the amount thereof is at least 1×10⁷, 2×10⁷, 3×10⁷, 4×10⁷, 5×10⁷, 6×10⁷, 7×10⁷, 8×10⁷, or 9×10⁷ VG per eye including all integers or fractional amounts within the range. In one embodiment, the amount thereof is at least 1×10⁸, 2×10⁸, 3×10⁸, 4×10⁸, 5×10⁸, 6×10⁸, 7×10⁸, 8×10⁸, or 9×10⁸ VG per eye including all integers or fractional amounts within the range. In one embodiment, the amount thereof is at least 1×10⁹, 2×10⁹, 3×10⁹, 4×10⁹, 5×10⁹, 6×10⁹, 7×10⁹, 8×10⁹, or 9×10⁹ VG per eye including all integers or fractional amounts within the range. In one embodiment, the amount thereof is at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ VG per eye including all integers or fractional amounts within the range. In one embodiment, the amount thereof is at least 1×10¹¹, 2×10¹¹, 3×10¹¹, 4×10¹¹, 5×10¹¹, 6×10¹¹, 7×10¹¹, 8×10¹¹, or 9×10¹¹ VG per eye including all integers or fractional amounts within the range. In one embodiment, the amount thereof is at least 1×10¹², 2×10¹², 3×10¹², 4×10¹², 5×10¹², 6×10¹², 7×10¹², 8×10¹², or 9×10¹² VG per eye including all integers or fractional amounts within the range. In one embodiment, the amount thereof is at least 1×10¹³, 2×10¹³, 3×10¹³, 4×10¹³, 5×10¹³, 6×10¹³, 7×10¹³, 8×10¹³, or 9×10¹³ VG per eye including all integers or fractional amounts within the range. In one embodiment, the amount thereof is at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ VG per eye including all integers or fractional amounts within the range. In one embodiment, the amount thereof is at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, the methods comprises dose ranging from 1×10⁹ to about 1×10¹³ VG per eye per dose including all integers or fractional amounts within the range. In another embodiment, the method comprises delivery of the vector in an aqueous suspension. In another embodiment, the method comprises administering the rAAV described herein in a dosage of from 1×10⁹ to 1×10¹³ GC in a volume about or at least 150 microliters, thereby restoring visual function in said subject. All dosages may be measured by any known method, including as measured by oqPCR or digital droplet PCR (ddPCR) as described in, e.g., M. Lock et al, Hum Gene Ther Methods. 2014 April; 25(2): 115-25. doi: 10.1089/hgtb.2013.131, which is incorporated herein by reference.

These above doses may be administered in a variety of volumes of carrier, excipient or buffer formulation, ranging from about 25 to about 1000 microliters, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is about 800 μL. In another embodiment, the volume is between about 150 and 800 μL. In another embodiment, the volume is between about 700 and 1000 μL. In another embodiment, the volume is between about 250 and 500 μL.

In one embodiment, the viral constructs may be delivered in doses of from at least 1×10⁹ to about least 1×10¹¹ GCs in volumes of about 1 μL to about 3 μL for small animal subjects, such as mice. For larger veterinary subjects having eyes about the same size as human eyes, the larger human dosages and volumes stated above are useful. See, e.g., Diehl et al, J. Applied Toxicology, 21:15-23 (2001) for a discussion of good practices for administration of substances to various veterinary animals. This document is incorporated herein by reference.

It is desirable that the lowest effective concentration of virus or other delivery vehicle be utilized in order to reduce the risk of undesirable effects, such as toxicity, retinal dysplasia and detachment. Still other dosages in these ranges may be selected by the attending physician, taking into account the physical state of the subject, preferably human, being treated, the age of the subject, the ocular condition and the degree to which the disorder, if progressive, has developed.

Yet another aspect described herein is a method for treating, retarding or halting progression of optic neuropathy or optic neuritis in a mammalian subject. In one embodiment, an rAAV carrying the SIRT1 or NRF2 native, modified or codon optimized sequence, preferably suspended in a physiologically compatible carrier, diluent, excipient and/or adjuvant, may be administered to a desired subject including a human subject. This method comprises administering to a subject in need thereof any of the nucleic acid sequences, expression cassettes, rAAV genomes, plasmids, vectors or rAAV vectors or compositions containing them. In one embodiment, the composition is delivered subretinally. In another embodiment, the composition is delivered intravitreally. In still another embodiment, the composition is delivered using a combination of administrative routes suitable for treatment of optic neuropathy or optic neuritis, and may also involve administration via the palpebral vein or other intravenous or conventional administration routes.

For use in these methods, the volume and viral titer of each dosage is determined individually, as further described herein, and may be the same or different from other treatments performed in the same, or contralateral, eye. The dosages, administrations and regimens may be determined by the attending physician given the teachings of this specification. In one embodiment, the composition is administered in a single dosage selected from those above listed in an affected eye. In another embodiment, the composition is administered as a single dosage selected from those above listed in a both affected eyes, either simultaneously or sequentially. Sequential administration may imply a time gap of administration from one eye to another from intervals of minutes, hours, days, weeks or months. In another embodiment, the method involves administering the compositions to an eye two or more dosages (e.g., split dosages). In another embodiment, multiple injections are made in different portions of the same eye. In another embodiment, a second administration of an rAAV including the selected expression cassette (e.g., SIRT1 or NRF2 containing cassette) is performed at a later time point. Such time point may be weeks, months or years following the first administration. Such second administration is, in one embodiment, performed with an rAAV having a different capsid than the rAAV from the first administration. In another embodiment, the rAAV from the first and second administration have the same capsid.

In still other embodiments, the compositions described herein may be delivered in a single composition or multiple compositions. Optionally, two or more different AAV may be delivered, or multiple viruses [see. e.g., WO 2011/126808 and WO 2013/049493]. In another embodiment, multiple viruses may contain different replication-defective viruses (e.g., AAV and adenovirus).

In certain embodiments of the invention, it is desirable to perform non-invasive retinal imaging and functional studies to identify areas of the ganglion cells to be targeted for therapy as well as to test the efficacy of treatment. In these embodiments, clinical diagnostic tests are employed to determine the precise location(s) for one or more subretinal injection(s). These tests may include electroretinography (ERG), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, Multi-electrode array (MEA), Pupillary Light Responses, etc, depending upon the species of the subject being treated, their physical status and health and the dosage. In view of the imaging and functional studies, in some embodiments of the invention one or more injections are performed in the same eye in order to target different areas of the affected eye. The volume and viral titer of each injection is determined individually, as further described herein, and may be the same or different from other injections performed in the same, or contralateral, eye. In another embodiment, a single, larger volume injection is made in order to treat the entire eye. In one embodiment, the volume and concentration of the rAAV composition is selected so that only the region of damaged ocular cells is impacted. In another embodiment, the volume and/or concentration of the rAAV composition is a greater amount, in order reach larger portions of the eye, including non-damaged ganglion cells.

In another embodiment, the method includes performing additional studies, e.g., functional and imaging studies to determine the efficacy of the treatment. For examination in animals, such tests include retinal and visual function assessment via electroretinograms (ERGs) looking at rod and cone photoreceptor function, optokinetic nystagmus, pupillometry, water maze testing, light-dark preference, optical coherence tomography (to measure thickness of various layers of the retina), histology (retinal thickness, rows of nuclei in the outer nuclear layer, immunofluorescence to document transgene expression, cone photoreceptor counting, staining of retinal sections with peanut agglutinin—which identifies cone photoreceptor sheaths).

Specifically for human subjects, following administration of a dosage of a composition described in this specification, the subject is tested for efficacy of treatment using electroretinograms (ERGs) to examine rod and cone photoreceptor function, pupillometry visual acuity, contrast sensitivity color vision testing, visual field testing (Humphrey visual fields/Goldmann visual fields), perimetry mobility test (obstacle course), and reading speed test. Other useful post-treatment efficacy test to which the subject is exposed following treatment with a pharmaceutical composition described herein are functional magnetic resonance imaging (fMRI), full-field light sensitivity testing, retinal structure studies including optical coherence tomography, fundus photography, fundus autofluorescence, adaptive optics laser scanning ophthalmoscopy, mobility testing, test of reading speed and accuracy, microperimetry and/or ophthalmoscopy. These and other efficacy tests are described in U.S. Pat. No. 8,147,823; in co-pending International patent application publication WO 2014/011210 or WO 2014/124282, incorporated by reference.

In one embodiment of the methods described herein, a one-time intra-ocular delivery of a composition as described herein, e.g., an AAV delivery of an hNRF2 cassette, is useful in treating optic neuropathy or optic neuritis in a subject. In another embodiment of the methods described herein, a one-time intra-ocular delivery of a composition as described herein, e.g., an AAV delivery of an hNRF2 cassette, is useful in treating optic neuropathy or optic neuritis in a subject at risk.

In one embodiment of the methods described herein, a one-time intra-ocular delivery of a composition as described herein, e.g., an AAV delivery of an hSIRT1 cassette, is useful in treating optic neuropathy or optic neuritis in a subject. In another embodiment of the methods described herein, a one-time intra-ocular delivery of a composition as described herein, e.g., an AAV delivery of an hSIRT1 cassette, is useful in treating optic neuropathy or optic neuritis in a subject at risk.

Thus, in one embodiment, the composition is administered before disease onset. In another embodiment, the composition is administered prior to the initiation of vision impairment or loss. In another embodiment, the composition is administered after initiation of vision impairment or loss. In yet another embodiment, the composition is administered when less than 90% of the RGCs are functioning or remaining, as compared to a non-diseased eye. In one embodiment, neonatal treatment is defined as being administered a SIRT1 or NRF2 coding sequence, expression cassette or vector as described herein within 8 hours, the first 12 hours, the first 24 hours, or the first 48 hours of delivery. In another embodiment, particularly for a primate (human or non-human), neonatal delivery is within the period of about 12 hours to about 1 week, 2 weeks, 3 weeks, or about 1 month, or after about 24 hours to about 48 hours. In another embodiment, the composition is delivered after onset of symptoms. In one embodiment, treatment of the patient (e.g., a first injection) is initiated prior to the first year of life. In another embodiment, treatment is initiated after the first 1 year, or after the first 2 to 3 years of age, after 5 years of age, after 11 years of age, or at an older age. In one embodiment, treatment is initiated from ages about 4 years of age to about 12 years of age. In one embodiment, treatment is initiated on or after about 4 years of age. In one embodiment, treatment is initiated on or after about 5 years of age. In one embodiment, treatment is initiated on or after about 6 years of age. In one embodiment, treatment is initiated on or after about 7 years of age. In one embodiment, treatment is initiated on or after about 8 years of age. In one embodiment, treatment is initiated on or after about 9 years of age. In one embodiment, treatment is initiated on or after about 10 years of age. In one embodiment, treatment is initiated on or after about 11 years of age. In one embodiment, treatment is initiated on or after about 12 years of age. However, treatment can be initiated on or after about 15, about 20, about 25, about 30, about 35, about 40, about 45, about 50, about 55, about 60, about 65 or about 70 years of age. In one embodiment, treatment in utero is defined as administering the composition as described herein in the fetus. See, e.g., David et al, Recombinant adeno-associated virus-mediated in utero gene transfer gives therapeutic transgene expression in the sheep, Hum Gene Ther. 2011 April; 22(4): 419-26. doi: 10.1089/hum.2010.007. Epub 2011 Feb. 2, which is incorporated herein by reference.

In another embodiment, the composition is readministered at a later date. Optionally, more than one readministration is permitted. Such readministration may be with the same type of vector, a different viral vector, or via non-viral delivery as described herein. In one embodiment, the vector is readministered to the patient to a different portion of the initially injected retina. In one embodiment, the vector is readministered to the patient to the same portion of the initially injected retina.

In yet another embodiment, any of the above described methods is performed in combination with another, or secondary, therapy. The secondary therapy may be any now known, or as yet unknown, therapy which helps prevent, arrest or ameliorate these mutations or defects or any of the effects associated therewith. The secondary therapy can be administered before, concurrent with, or after administration of the compositions described above. In one embodiment, a secondary therapy involves non-specific approaches for maintaining the health of the retinal cells, such as administration of neurotrophic factors, anti-oxidants, anti-apoptotic agents. The non-specific approaches are achieved through injection of proteins, recombinant DNA, recombinant viral vectors, stem cells, fetal tissue, or genetically modified cells. The latter could include genetically modified cells that are encapsulated.

In one embodiment, a method of generating a recombinant rAAV comprises obtaining a plasmid containing an AAV expression cassette as described above and culturing a packaging cell carrying the plasmid in the presence of sufficient viral sequences to permit packaging of the AAV viral genome into an infectious AAV envelope or capsid. Specific methods of rAAV vector generation are described above and may be employed in generating a rAAV vector that can deliver the SIRT1 or NRF2 coding sequence in the expression cassettes and genomes described above and in the examples below.

In certain embodiments of this invention, a subject has optic neuropathy or optic neuritis or other ocular condition described herein, for which the components, compositions and methods of this invention are designed to treat. As used herein, the term “subject” as used herein means a mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, non-human primate and others. As used herein, the term “subject” is used interchangeably with “patient”.

As used herein, the term “treatment” or “treating” is defined encompassing administering to a subject one or more compounds or compositions described herein for the purposes of amelioration of one or more symptoms of the ocular disorders described herein. “Treatment” can thus include one or more of reducing onset or progression of the ocular disorders described herein, preventing disease, reducing the severity of the disease symptoms, or retarding their progression, including the progression of blindness, removing the disease symptoms, delaying onset of disease or monitoring progression of disease or efficacy of therapy in a given subject.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, “disease”, “disorder” and “condition” are used interchangeably, to indicate an abnormal state in a subject.

As used herein, the term “about” or “˜” means a variability of 10% from the reference given, unless otherwise specified.

The term “regulation” or variations thereof as used herein refers to the ability of a composition to inhibit one or more components of a biological pathway.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

The following examples are illustrative only and are not intended to limit the present invention.

### Example 1: Recombinant rAAV and In Vitro Expression Studies

Here we describe several agents which can be delivered to retinal ganglion cells and potentially prevent permanent vision loss. The agents, NRF2 and Sirtuins (SIRT1, 2, 3, 4 5, 6, 7) act by decreasing oxidative or metabolic stress and thus by providing neurotrophic support. Sirtuins are NAD-dependent protein deacetylases. Intravitreal delivery of sirtuin activators such as resveratrol have been shown to reduce deficits from optic neuritis and optic crush injury in mouse models. (Shindler et al. Invest Ophthalmol Vis Sci 48(8):3602 (2007): Zuo et al Invest Ophthalmol Vis Sci 54(7):5097-102 (2013)). Nuclear factor-like 2 (NRF2) is a transcription factor that regulates a pathway of genes that decrease oxidative and other forms of stress.

We have generated rAAV vectors capable of delivering NRF2 (FIG. 1) and sirtuins (SIRT1: FIG. 2) to diseased tissue, including retinal cells in the eye. The vectors are used to treat a wide variety of genetic and acquired optic nerve disorders. Our results indicate that delivery of the proviral plasmid or of a recombinant adeno-associated virus (AAV) containing the NRF2 cDNA to eyes of mice leads to expression of NRF2 and in animals with optic neuritis, can ameliorate the disorder.

### Example 2: Vector Design

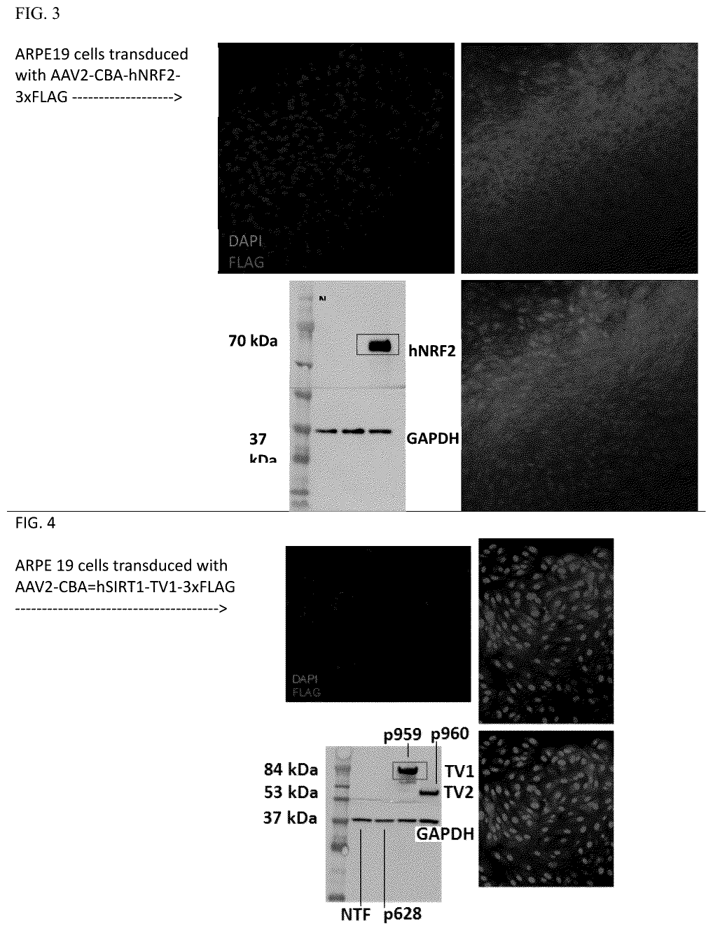

Cis plasmids including the hNRF2 (FIG. 1) or hSIRT1 (FIG. 2) coding sequences were designed, pAAV-CMV/CBA-hNRF2-3×FLAG (SEQ ID NO: 5) uses the chicken beta actin (CBA) promoter plus cytomegalovirus (CMV) enhancer to drive expression of the human NRF2 cDNA with a C-terminal 3×FLAG epitope tag. (CBA) promoter. The NRF2 sequence terminates into a bovine growth hormone (bGH) polyadenylation signal. The entire AAV expression cassette is flanked by the AAV2 inverted terminal repeats (ITRs). Transfection of ARPE19 cells resulted in both immunohistochemically detectable transgene expression and production of NRF2 protein of the predicted size. FIG. 3.

pAAV-CMV/CBA-hSIRT1-3×FLAG (SEQ ID NO: 6) uses the chicken beta actin (CBA) promoter plus cytomegalovirus (CMV) enhancer to drive expression of the human SIRT1 cDNA with a C-terminal 3×FLAG epitope tag. (CBA) promoter. The SIRT1 sequence terminates into a bovine growth hormone (bGH) polyadenylation signal. The entire AAV expression cassette is flanked by the AAV2 inverted terminal repeats (ITRs). Transfection of ARPE19 cells resulted in both immunohistochemically detectable transgene expression and production of SIRT1 protein of the predicted size. FIG. 4.

### Example 3: Transfection of Neuro2a Cells

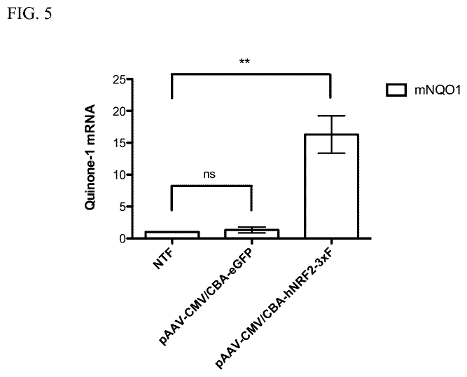

Transfection with pAAV-CMV/CBA-hNRF2-3×FLAG results in gene activation in Neuro2a cells (a neuroblastoma cell line). pAAV-CMV/CBA-eGFP was transfected as control in a second cohort of cells. RNA was extracted at 48 hours post-transfection and synthesized into cDNA. qRT-PCR analysis of quinone-1 (mNQO1) mRNA (a major downstream transcriptional target of NRF2) reveals enriched transcripts levels (**=P<0.01) in the pAAV-CMV/CBA-hNRF2-3×FLAG-treated cells compared to nontransfected and reporter transfected cells. FIG. 5.

### Example 4: Injection of Mouse Model

A mouse model of experimental optic neuritis was tested to see if injection with AAV-hNRF2 or AAVhSIRT1 vectors preserved RGC function.

Wild-type mice receive intravitreal injection of AAV2 or AAV7m8 vectors at P30 (2 μL, 2E10 vg). EAE phenotype induced by injection of MOG35-39 emulsified in CFA at two sites on the back followed by IP injection of pertussis toxin. MOG+CFA induces autoimmune response while PT degrades blood-brain barrier, allowing immune entry to the CNS.

Sham-induced animals receive injections of PBS+CFA and PT. Baseline OKR measured prior to EAE induction (week 0) and once a week for 7 weeks.

Cohort 1=10 control mice (sham-induced) were injected as follows: OD=AAV2-CAG-eGFP and OS=vehicle.

Cohort 2=10 control mice (EAE-induced) were injected as follows: OD=AAV2-CAG-eGFP and OS=vehicle.

Cohort 3: 25 mice (EAE-induced) were injected as follows: OD (oculus dextrus)=AAV2-CAG-hNRF2-3×FLAG and OS (oculus sinister)=AAV2-CAG-hSIRT1-3×FLAG

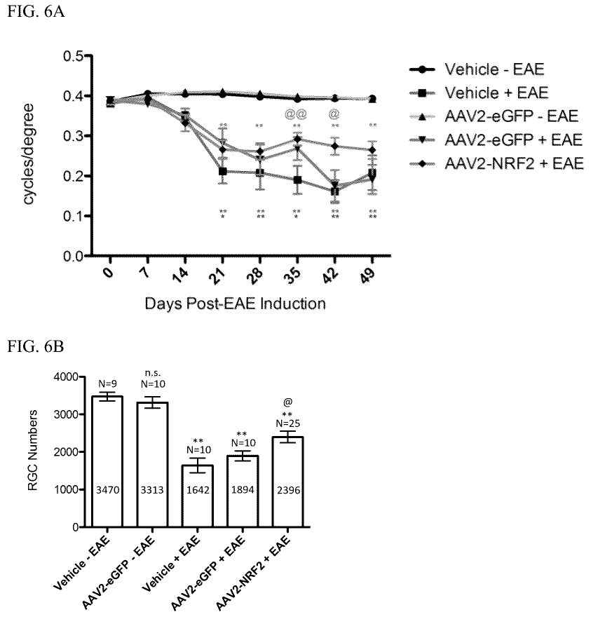

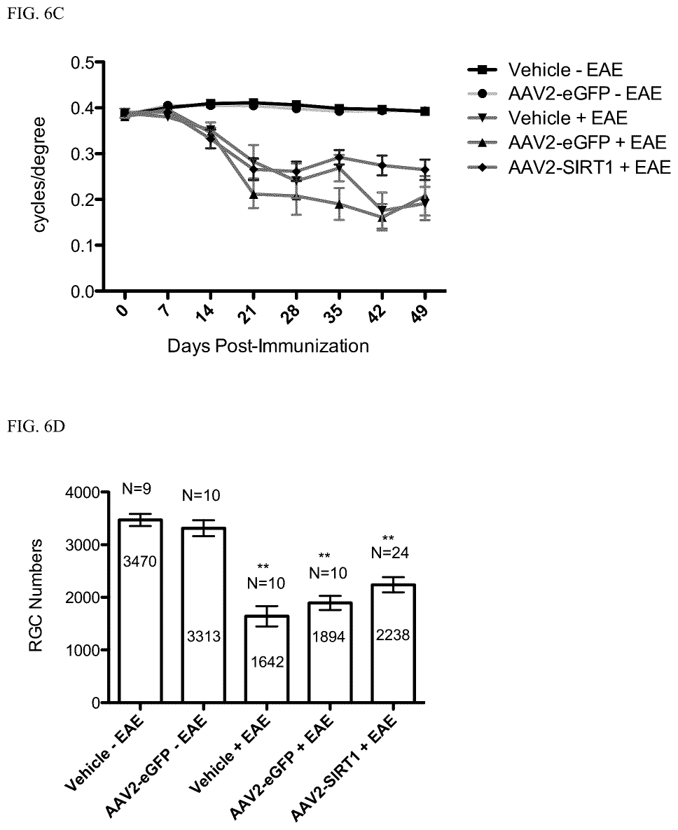

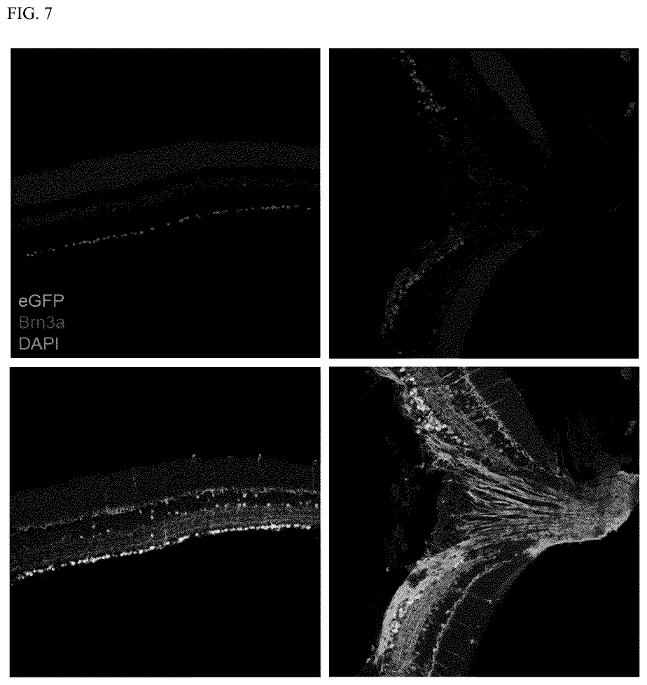

Intravitreal injection of AAV2-CMV/CBA-hNRF2 or AAV2-CMV/CBA-hSIRT1 resulted in preservation of retinal ganglion cell (RGC) function compared to controls. Retinal function was assessed in a masked fashion by measuring the optokinetic reflex (OKR), a measure of visual acuity. The greater the cycles/degree, the better the visual acuity. Sham-induced mice treated with vehicle or AAV2-eGFP exhibit robust OKR scores throughout the experiment. EAE-induced mice show a significant decline in visual function at experimental day 21 and throughout the remainder of the experiment, mirroring the inflammation and cell death occurring in the optic nerve. However, EAE-induced animals treated with AAV2-NRF2 exhibit significantly enhanced OKR scores at day 35 (@@): P<0.01) and day 42 (@): P<0.05) and demonstrate a trend towards improvement at days 21, 28, and 49 compared to EAE-induced animals treated with vehicle. FIG. 6A. EAE-induced animals treated with AAV2-SIRT1 exhibit statistically significant preservation in OKR compared to AAV2-eGFP+EAE at day 35 (P<0.01) and day 42 (P<0.05) and compared to EAE-induced animals treated with vehicle. FIG. 6C.

Intravitreal injection of AAV2-CMV/CBA-hNRF2 or AAV2-CMV/CBA-hSIRT1 in a mouse model of experimental optic neuritis results in preservation of retinal ganglion cell (RGC) numbers compared to controls. Retinal ganglion cell numbers were assessed in a masked fashion by histology. EAE immunized animals exhibit significantly reduced RGC numbers compared to non-induced controls injected with vehicle (**=P<0.01). However, treatment with AAV2-CMV/CBA-hNRF2-3×FLAG or AAV2-CMV/CBA-hNRF2-3×SIRT1 provides significantly enhanced RGC survival compared to EAE immunized controls injected with vehicle (Ω=P<0.05). FIG. 6B, 6D.

Neither NRF2 nor SIRT1 overexpression can halt optic nerve demyelination.

### Example 5: SIRT1 Gene Transfer Promotes Retinal Ganglion Cell Neuroprotection in Experimental Optic Neuritis

The following describes experiments detailed in McDougald et al, Investigative Ophthalmology & Visual Science March 2018, Vol. 59, 1212-1220, which is incorporated herein by reference in its entirety.

Multiple sclerosis (MS) is a chronic, inflammatory disease characterized by activation of CD4+ T cells that infiltrate the central nervous system (CNS) and mount an autoimmune response against myelin. Optic neuritis is a condition commonly observed in MS patients that leads to temporary or permanent visual decline following demyelination of the optic nerve and loss of retinal ganglion cells. Current therapies for MS and ON include immunosuppressive agents that mitigate the inflammatory component of disease. Unfortunately, these treatments provide temporary symptomatic relief and, moreover, do not attenuate further neuronal loss. Therefore, it is critical to identify alternative treatment strategies that address underlying mechanisms of neuropathology. The conserved role of oxidative injury in MS and other forms of neurodegenerative disease is an attractive therapeutic target to delay or halt disease progression. Here we describe an approach that preserves retinal ganglion cell numbers and function during experimental optic neuritis following SIRT1 gene augmentation. SIRT1 is an NAD-dependent deacetylase that activates numerous cytoprotective mechanisms that suppress ROS activity, improve mitochondrial function, and inhibit apoptosis. We generated and characterized adeno-associated virus (AAV) vectors that drive constitutive expression of human SIRT1 using retinal-derived cell lines and in vivo models. Wild-type mice received intravitreal injections of AAV-SIRT1, AAV-eGFP, or vehicle. Afterwards, mice were vaccinated with myelin antigen to induce experimental autoimmune encephalomyelitis (EAE), an established model of MS that recapitulates the clinical features of optic neuritis including reduced visual acuity, optic nerve atrophy, and death of retinal ganglion cells (RGCs). Ganglion cell function was evaluated following EAE induction by measuring the optokinetic response (OKR). All EAE induced animals exhibited severely reduced OKR scores compared to sham immunized controls. However, treatment with AAV-SIRT1 improved visual acuity compared to vehicle and reporter injected animals also subjected to EAE. To examine the effect of SIRT1 gene augmentation on neuronal survival, retinae were harvested and stained with antibodies to label and quantify ganglion cells. Treatment with AAV-SIRT1 increased RGC survival compared to vehicle and reporter treated controls. Ongoing experiments will examine the contribution of SIRT1 gene augmentation in mitigating optic nerve atrophy as well as restricting oxidative damage in affected cell types. Collectively, this investigation suggests AAV-mediated SIRT1 gene augmentation can mediate neuroprotection in optic neuritis and MS pathogenesis.

A. Current understanding of MS pathogenesis has benefited from studies of experimental autoimmune encephalomyelitis (EAE).6 Manifestations of optic neuritis in EAE include optic nerve thinning. RGC loss, and reduced visual function, similar to the human disease, thus providing an in vivo system for characterization of neurodegenerative processes and a platform for interrogating neuroprotective strategies.7-9 Prior studies suggest oxidative stress and mitochondrial injury are central mediators of MS pathology. 1 Accumulation of reactive oxygen and nitrogen species (ROS/RNS) within MS lesions leads to damage of cellular components including proteins, lipids, and DNA. Fortunately, eukaryotic cells are equipped with a collection of defense systems to combat oxidative injury and maintain redox homeostasis. 10,11 We hypothesized that amplifying such mechanisms within RGCs using a conventional gene transfer approach may promote neuroprotection in experimental optic neuritis.

Nuclear factor (erythroid-derived 2)-like 2 (NRF2) is a basic leucine zipper transcription factor that activates a network of genes associated with antioxidant defense and cellular detoxification. 11 Transgenic ablation of NRF2 during EAE development generates a phenotype of accelerated demyelination, immune cell infiltration, and proinflammatory cytokine signaling compared to wild-type animals also subjected to EAE. 12 In addition, Nrf2 knockout mice demonstrate enhanced decline in visual function, loss of RGCs, and exacerbated optic nerve atrophy. 13 Accumulating evidence supports therapeutic modulation of NRF2 activity via small molecule activation or transgenic overexpression in neurodegenerative diseases driven by oxidation. 14-17 A gene augmentation strategy demonstrated a transient increase in survival of RGCs targeted with NRF2 expression vectors following optic nerve crush. 17 Based on the collective data, we reasoned that stimulation of NRF2 activity may provide an effective means to protect RGCs in optic neuritis.

Sirtuin 1 (SIRT1) is an evolutionarily conserved NAD+-dependent deacetylase that regulates various components of cellular metabolism with respect to aging, DNA repair, mitochondrial biogenesis, and apoptosis. 18 Accumulating evidence suggests modulation of SIRT1 activity via pharmacologic induction or transgenic overexpression may offer therapeutic value in several forms of neurodegenerative disease. 19-27 In experimental optic neuritis, small molecule activators of SIRT1, including resveratrol and related polyphenolic compounds, are effective in preserving visual acuity and RGC survival during EAE and viral-induced demyelinating disease. 19,21 In addition, Nimmagadda et al.24 demonstrated suppression of inflammation and demyelination following EAE sensitization using a transgenic mouse containing neural-restricted overexpression of SIRT1. However, the study design was limited to MS lesions localized to the spinal cord and did not examine the contribution of SIRT1 overexpression in ameliorating ocular disease manifestations.24 Potential neuroprotective effects of SIRT1 overexpression specifically in RGCs need to be examined.

In the present study, we interrogated the effects of SIRT1 or NRF2 overexpression in experimental optic neuritis via adeno-associated virus (AAV) gene transfer to RGCs. We developed and characterized AAV serotype 2 (AAV2) vectors that drive constitutive expression of human NRF2 and SIRT1 in vitro and in the mouse retina. We examined the neuroprotective contribution of SIRT1 and NRF2 gene augmentation in suppressing RGC death, optic nerve inflammation and demyelination, and vision loss in EAE mice.

### B. Methods

### Animals

C57Bl/6J mice were obtained from the Jackson Laboratory and raised in a 12-hour light/dark cycle. Animals were housed at the University of Pennsylvania in compliance with ARVO Statement for the Use of Animals in Ophthalmic and Vision Research as well as with institutional and federal regulations.

### AAV Vector Design and Production

Human SIRT1 (transcript variant 1) and human NRF2 (transcript variant 1) cDNA clones were obtained from Origene. Sequences were amplified with Q5 DNA polymerase (NEB) and cloned into an AAV expression plasmid using a commercial cloning kit (In-Fusion HD; Clontech Laboratories, Mountain View, CA, USA). Transgene expression was driven by a CAG promoter derived from pDRIVE-CAG (InvivoGen, San Diego, CA, USA). Both cDNA sequences contained a C-terminal 3×FLAG epitope tag that terminates into a bovine growth hormone (bGH) polyadenylation sequence. AAV expression cassettes were flanked by the AAV2 inverted terminal repeats. A proviral plasmid driving expression of enhanced green fluorescent protein (eGFP) was obtained from Jean Bennett, MD, PhD (University of Pennsylvania) and contains identical cis regulatory elements. AAV2-NRF2, AAV2-SIRT1, and AAV2-eGFP vectors were generated using previously described methods and purified with CsCl gradient by the CAROT research vector core at the University of Pennsylvania.

### Cell Culture

ARPE-19 cells were supplied by ATCC (Manassas, VA, USA) and grown at 37° C. with 5% CO2. Cells were maintained in Dulbecco's modified Eagle's medium: nutrient mixture F-12 (DMEM/F12; Gibco Laboratories, Gaithersburg, MD, USA) and supplemented with 10% fetal bovine serum (FBS) and 1% penicillin-streptomycin. 84-31 cells were provided by James Wilson, MD, PhD (University of Pennsylvania) and were cultured in medium (DMEM-GlutaMax; Gibco Laboratories) and supplemented with 10% FBS and 1% penicillin-streptomycin. We seeded 84-31 cells at a density of 350,000 cells and transduced with AAV2 vectors at a multiplicity of infection (MOI) of 100,000. Cells were harvested for expression analysis at 48 hours posttransduction. For AAV transduction in ARPE-19 cells, 150,000 cells were plated and transduced with AAV2 vectors at an MOI of 100,000. Cells were harvested for expression analysis at 72 hours posttransduction. Cells were rinsed with PBS and fixed in 4% paraformaldehyde for 15 minutes at room temperature. Afterwards, cells were blocked in 0.1% Triton X-100 and 1% bovine serum albumin (BSA) for 30 minutes at room temperature. Cells were incubated with primary antibody solution containing 1% BSA and rabbit anti-FLAG antibody (CST #14793; 1:200) for 1 hour at room temperature. Cells were washed with PBS and incubated in secondary antibody solution containing 1% BSA and goat anti-rabbit AlexaFluor-594 antibodies (1:500) for 1 hour at room temperature. Cells were removed from secondary incubation, washed in PBS, and mounted with (Fluoromount-G; Southern Biotech; Birmingham, AL, USA) containing DAPI.

### Quantitative Real-Time PCR (RT-qPCR)

RNA was isolated from 84-31 cells (provided by James Wilson) using the RNA kit (Macherey-Nagel Nucleospin RNA kit: Thermo Fisher Scientific, Waltham, MA, USA). First-strand cDNA synthesis was performed using 500 ng of total RNA with the first-strand synthesis system (SuperScript III: Thermo Fisher Scientific) according to manufacturer's protocol. Real-time PCR was performed with a commercial system (7500 Fast: Applied Biosystems, Foster City, CA, USA) using a PCR master mix (Power SYBR green: Invitrogen). The following primer sequences were used: 5′ CCACTCCTCCACCTTTGAC 3′ (human GAPDH Forward): 5′ ACCCTGTTGCTGTAGCCA 3′ (human GAPDH Reverse); GAGCTGGGGTGTCTGTTTCA (human SIRT1 Forward); GGAAGTCTACAGCAAGGCGA (human SIRT1 Reverse); GTCACATCGAGAGCCCAGTC (human NRF2 Forward); and AGCTCCTCCCAAACTTGCTC (human NRF2 Reverse). Relative gene expression was quantified with the ΔΔCT method and normalized to GAPDH.

### Intravitreal Injections

We anesthetized 4-week-old mice by isoflurane inhalation. A 33½ gauge needle was used to create a small incision at the limbus. Afterward, a 10-μL Hamilton syringe (701 RN; Hamilton Company, Reno, NV, USA) attached to a 33-gauge blunt-end needle was inserted into the vitreous cavity with the needle tip placed directly above the optic nerve head. We dispensed 2 μL of AAV preparation containing approximately 1×10¹⁰ vector genomes were dispensed into each eye bilaterally. Vehicle treated eyes were injected with an equivalent volume of vector dilution buffer (0.001% Pluronic F68 in PBS). The two eyes of each mouse received different injections (vehicle, AAV2-NRF2, AAV2-SIRT1, or AAV2-eGFP) allowing each eye to serve as an independent experimental end point.

### Induction and Score of EAE

We anesthetized 8-week-old C57Bl/6 mice by isoflurane inhalation and injected at two sites subcutaneously with 200 μg of myelin oligodendrocyte glycoprotein peptide (MOG35-55; GenScript, Piscataway, NJ, USA) emulsified in antigen solution (Complete Freund's Adjuvant [CFA]; Difco Laboratories, Inc., Detroit, MI, USA) with 2.5 mg/mL Mycobacterium tuberculosis (Difco Laboratories, Inc.). Control mice that were not induced for EAE were injected with an equal volume of PBS and CFA. All mice were given 200 ng pertussis toxin (List Biological, Campbell, CA, USA) in 0.1 mL of PBS by intraperitoneal injection at 0 hours and 48 hours postimmunization with MOG35-55. Clinical EAE was assessed using a previously described five-point scale 19: no disease=0; partial tail paralysis=0.5: tail paralysis or waddling gait=1.0: partial tail paralysis and waddling gait=1.5: tail paralysis and waddling gait=2.0: partial limb paralysis=2.5: paralysis of one limb=3.0: paralysis of one limb and partial paralysis of another=3.5: paralysis of two limbs=4.0; moribund state=4.5; death=5.0.

### Optokinetic Response Recordings (OKRs)

Visual function was assessed by measuring the OKR using commercial software and apparatus (OptoMotry: CerebralMechanics, Inc., Medicine Hat, AB, Canada) as previously described.28 OKR was determined as the highest spatial frequency where mice track a 100% contract grating that is projected at different spatial frequencies. Measurements were performed by an investigator blinded to the experimental treatments.

### Retinal Histology and RGC Quantification

Eyes were harvested and placed in 4% paraformaldehyde (PFA) overnight at 4° C. Eyes were washed in PBS followed by dissection of retinal cups. Tissues were permeabilized and blocked in 2% Triton X-100, 10% normal donkey serum, and PBS and then incubated with goat anti-Brn3a antibody (Santa Cruz Biotechnology, Dallas, TX, USA) diluted 1:100 at 4° C. Retinal cups were washed and then incubated in secondary antibody solution containing 2% Triton X-100, 10% normal donkey serum, and donkey anti-goat AlexaFluor 594 antibody (1:500 dilution). After washing, samples were prepared as flatmounts and mounted onto glass slides with an aqueous mounting medium (SouthernBiotech) containing 4′,6-diamidino-2-phenylindole (DAPI). RGCs were quantified as previously described.7,19,25,26 Briefly, retinal micrographs were recorded at ×40 magnification in 12 standard fields (⅙, 3/6, and ⅚ of the retinal radius from the center of the retina in each quadrant). Total RGC counts from the 12 fields per retinal sample covering a total area of 0.45 mm2/retina were recorded by an investigator masked to the experimental conditions using ImageJ software (available at imagej.nih.gov/ij/; provided in the public domain by the National Institutes of Health, Bethesda, MD, USA). Retinal cross-sections were incubated in blocking buffer containing PBS, 2% Triton X-100, and 10% normal donkey serum for 1 hour at room temperature. Next, sections were incubated in primary antibody solution containing the previously described components and a rabbit anti-FLAG antibody (CST #14793) at 1:100 dilutions overnight in a humidified chamber at room temperature. Sections were washed in PBS three times and incubated in secondary antibody solution containing donkey anti-rabbit AlexaFluor 488 antibody diluted at 1:200 for 2 hours at room temperature. Slides were then washed in PBS three times and mounted with aqueous mounting medium (SouthernBiotech) containing DAPI.

### Optic Nerve Histology and Scoring

Histologic staining and scoring was performed as in prior studies. 7-9,19-25 Optic nerves were harvested, fixed in 4% PFA, and embedded in paraffin. Nerves were subsequently cut into 5-μm longitudinal sections. To examine immune cell infiltration, sections were stained with hematoxylin and eosin (H&E). Inflammation was scored by an investigator blinded to the experimental treatments, and nerves were graded on a 0 to 4 point scale: no infiltration=0; mild cellular infiltration=1; moderate infiltration=2; severe infiltration=3; massive infiltration=4. Sections were stained with luxol fast blue (LFB) to assess myelination. These sections were graded on a 0 to 3 point scale: 0=no demyelination; 1=scattered foci of demyelination; 2=prominent foci of demyelination; and 3=large (confluent) areas of demyelination.

### Statistics

All data are represented as means±SEM. Differences between treatment groups with respect to OKR responses, RGC quantification, and optic nerve histopathology were compared using a 1-way ANOVA followed by Tukey's honest significant difference test using statistical software (GraphPad Prism 7.0; GraphPad Software, Inc., La Jolla, CA, USA). Differences were considered statistically significant at P<0.05.

### C. Results

### Design and Characterization of AAV2 Vectors

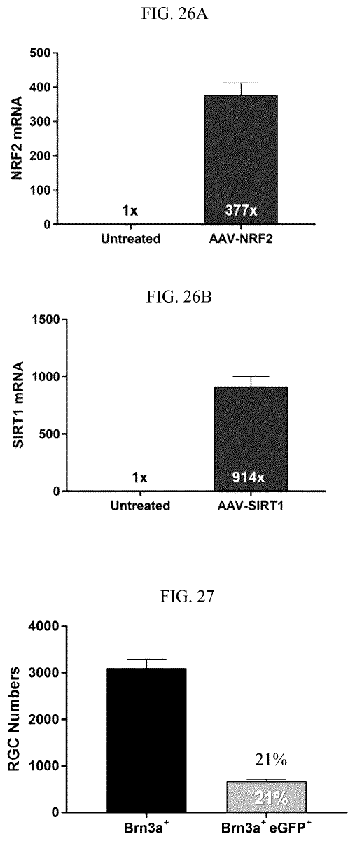

Vectors based upon recombinant adeno-associated virus (AAV) have emerged as the current standard for achieving safe and stable gene transfer directed to nondividing cells such as neurons. AAV serotype 2 (AAV2) demonstrates a robust safety profile following subretinal delivery in clinical trials for Leber congenital amaurosis type 2.29-32 We generated AAV2 vectors expressing eGFP, human NRF2, or human SIRT1 driven by a ubiquitous promoter (FIGS. 1 and 2). Vector expression was examined in vitro with RT-qPCR and immunofluorescence (Data not shown). RT-qPCR revealed robust levels of transgene expression in 84-31 cells treated with the designated vector compared to untreated controls (FIGS. 26A and 26B). Immunofluorescent labeling of ARPE-19 cells transduced with AAV2-SIRT1 demonstrates strong nuclear localization of the transgene product (Data not shown), while cells transduced with AAV2-NRF2 display robust cytoplasmic and nuclear distribution of the tagged protein (Data not shown). Next, we examined the retinal transduction profile of AAV2 following intravitreal delivery with a vector expressing enhanced green fluorescent protein in a cohort of wild-type mice. Similar to previously described reports, 33-38 AAV2-eGFP displayed transduction of the ganglion cell layer and optic nerve head Data not shown). This vector achieved approximately 21% RGC transduction by quantifying the number of eGFP positive RGCs labeled with Brn3a antibody (FIG. 27). AAV2 vectors driving expression of NRF2 or SIRT1 were injected into the right and left eyes, respectively, of wild-type mice display similar transduction profiles in vivo (Data not shown).

### SIRT1 Gene Transfer Preserves RGC Function During EAE

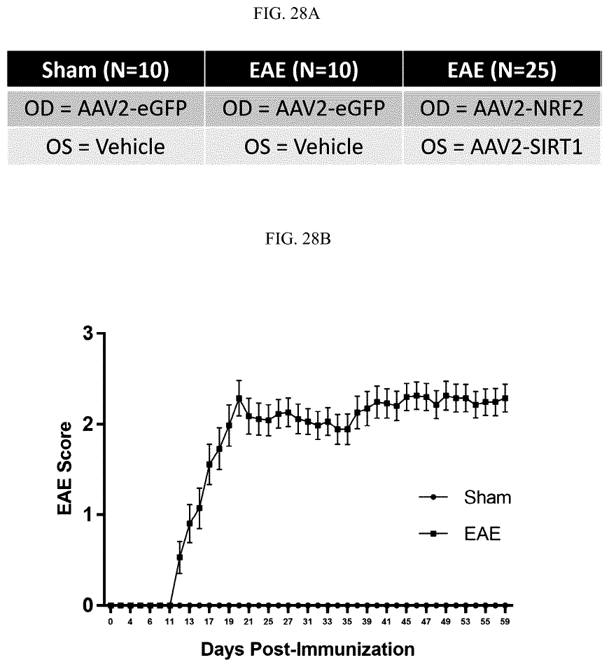

C57B16/J mice received intravitreal injections of AAV2 vectors or vehicle at postnatal week 4 followed by EAE/sham induction at postnatal week 8 (FIG. 28A). Following MOG35-55 immunization, animals displayed phenotypic features of EAE beginning near day 12 postimmunization (FIG. 28B) similar to prior studies. 7,19,25 We measured visual function in response to gene transfer by recording the OKR prior to EAE/sham immunization and once every 7 days postimmunization. Earlier reports demonstrate a marked reduction in the OKR throughout the course of EAE.7 Sham-induced animals treated with intravitreal injections of vehicle or AAV2-eGFP exhibit robust OKR scores throughout the experimental timeline, suggesting minimal adverse effects associated with intravitreal delivery, vector recruitment, and transgene overexpression. Similarly, animals injected with AAV2-NRF2 or AAV2-SIRT1 displayed strong responses prior to induction. Following EAE sensitization, MOG-induced animals exhibit a decline in OKR scores beginning by day 21 postinduction. However, eyes treated with AAV2-SIRT1 demonstrate an upward trend in functional responses throughout the experimental timeline. In addition, the AAV2-SIRT1 treatment group achieves statistically significant preservation at days 35 (AAV2-SIRT1=0.292±0.016: AAV2-eGFP=0.19=0.035; P=0.032) and 42 (AAV2-SIRT1=0.274±0.022: AAV2-eGFP=0.161±0.029: P=0.049) when compared to the EAE-induced control group treated with AAV2-eGFP. NRF2 augmentation did not provide statistically meaningful preservation of visual acuity throughout the experimental timeline.

### NRF2 Gene Transfer Improves RGC Survival During EAE

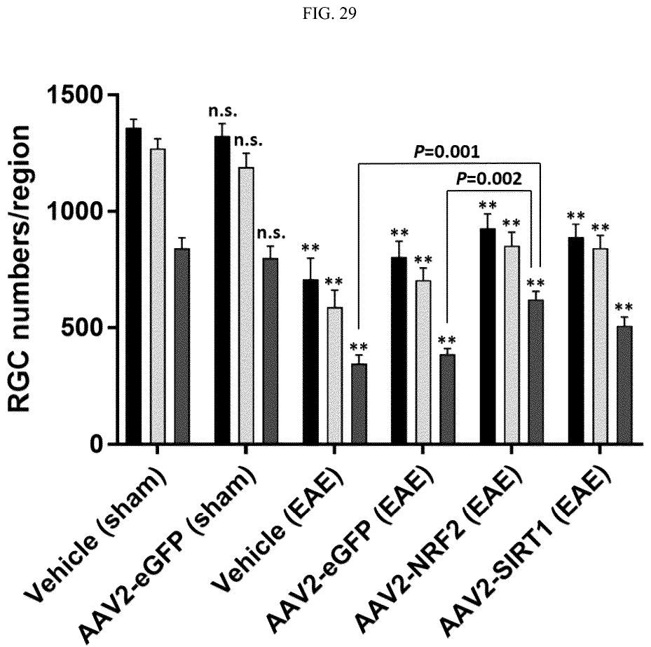

Permanent visual decline in optic neuritis coincides with the loss of RGCs.7 Retinas from each treatment group were isolated and stained with antibodies directed against Brn3a, a marker of RGCs, to determine whether SIRT1 or NRF2 gene augmentation conferred a protective advantage upon RGCs during EAE (FIG. 29). Intravitreal injection of AAV2 was well tolerated as indicated by comparative total RGC counts in sham-induced animals treated with vehicle. In mice sensitized to EAE, RGC numbers were significantly reduced in all treatment groups compared to sham-induced controls injected with vehicle or AAV2-eGFP (P<0.01). Treatment with AAV2-SIRT1 showed an upward trend in total RGC survival compared to control eyes, although this effect was not statistically significant. NRF2 gene transfer did provide a statistically significant increase in RGC survival compared to eyes treated with vehicle (P=0.027: FIG. 6B). We also examined the effect of NRF2 or SIRT1 gene transfer upon regional RGC density in the mouse retina (central, midperipheral, and peripheral: FIG. 29). NRF2 augmentation promoted survival of RGCs located within peripheral regions of the retina compared to both vehicle (P=0.001) and AAV2-eGFP (P=0.002) treatment groups sensitized to EAE. We observed a nonsignificant but trending increase in regional RGC density in retinas treated with AAV2-SIRT1.

Gene Transfer with NRF2 or SIRT1 Fails to Attenuate Optic Nerve Inflammation and Demyelination

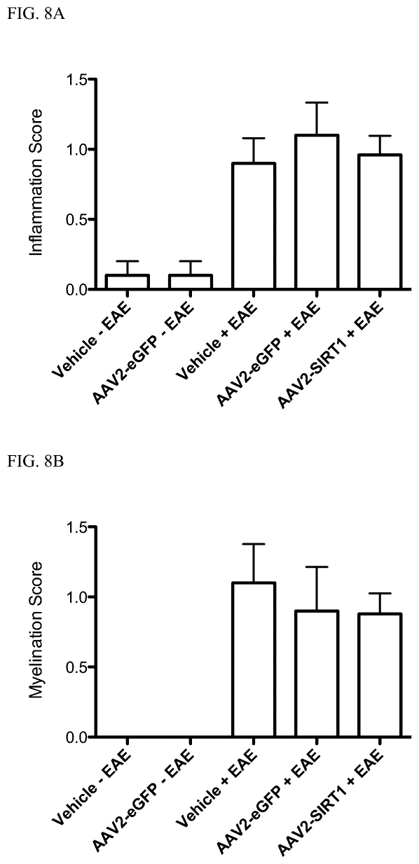

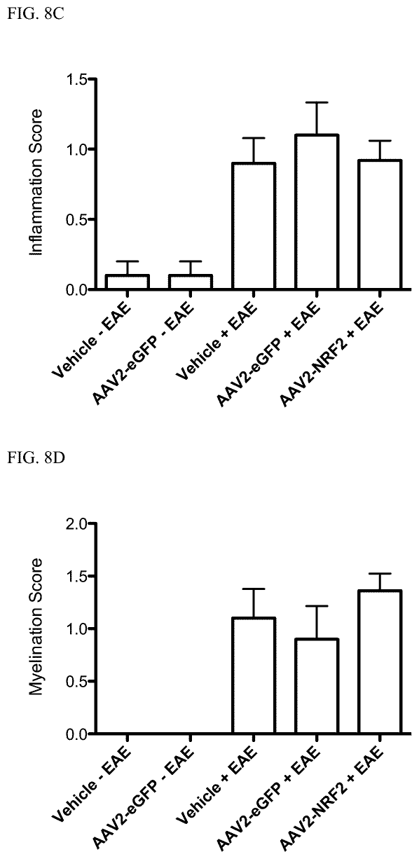

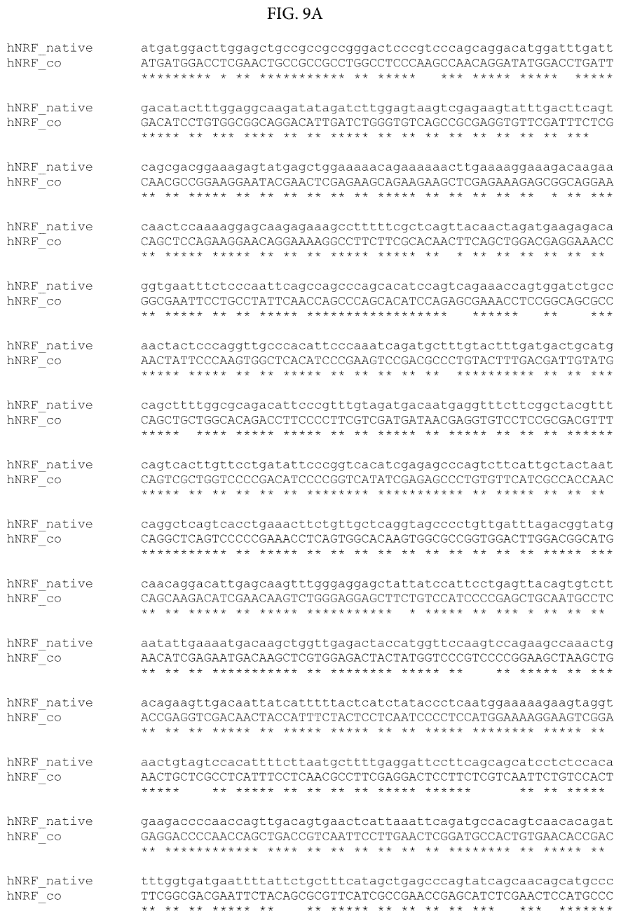

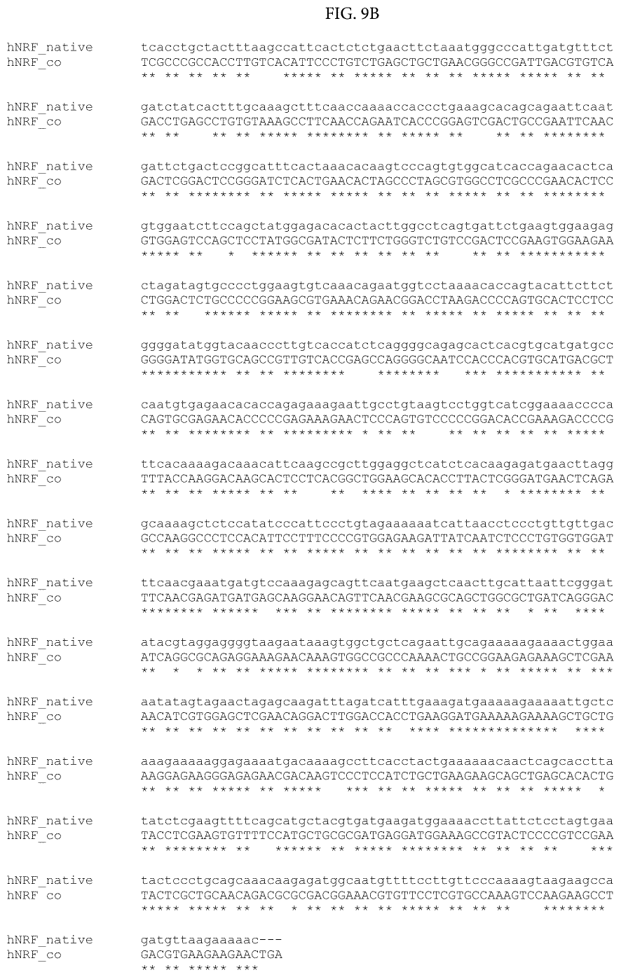

We investigated immune infiltration of the optic nerve in response to SIRT1 or NRF2 gene therapy. Optic nerve sections were subjected to H&E staining for evidence of immune cell infiltration. Optic nerves from sham-induced animals that received intravitreal injection of vehicle or AAV2-eGFP displayed minimal evidence of immune recruitment. However, all EAE-sensitized animal cohorts demonstrated enhanced infiltration. Optic nerves derived from animals dosed with NRF2 or SIRT1 vectors did not show a difference in immune recruitment compared to the vehicle and AAV2-eGFP treated animals undergoing EAE (FIG. 8A, C). We examined the effect of gene transfer on EAE-induced optic nerve demyelination by staining optic nerve sections with LFB. Sections from sham-induced animals injected with vehicle or AAV2-eGFP exhibited robust LFB labeling indicative of healthy nerves not subject to the immune-mediated demyelination of EAE. EAE animals treated with vehicle or AAV2-eGFP demonstrated decreased LFB staining compared to sham-induced animals. Gene transfer with SIRT1 or NRF2 did not prevent demyelination as these animals demonstrated similar myelination scores as EAE induced controls (FIG. 8B, D).

### D. Discussion

The present study explored the effects of SIRT1 or NRF2 gene transfer in experimental optic neuritis. Under cellular conditions of redox equilibrium, NRF2 is sequestered within the cytoplasm and subject to proteasomal-mediated degradation. 10,39,40 During oxidative challenge, modifications to critical binding proteins free NRF2 to translocate into the nucleus, recruit transcriptional machinery to antioxidant response elements (AREs), and stimulate transcription of target genes associated with antioxidant defense and cellular detoxification. 11 SIRT1 is recruited to the nucleus and other cellular compartments where it modulates the activity of various protein targets. SIRT1 is known to deacetylate and inhibit the transcription factor, p53, thereby downregulating apoptotic gene expression and thus improving cell viability 41 SIRT1 promotes mitochondrial function and antioxidant metabolism by activating PGC-1α, a master transcriptional regulator of these responses.42 While SIRT1 and NRF2 are typically believed to function via separate pathways, recent evidence suggests SIRT1 involvement in regulating the expression and activation of NRF2.43 In addition, treatment with pharmacologic agents such as resveratrol, a known activator of SIRT1, was shown to enhance NRF2 expression and activity of its downstream effectors.44,45 We hypothesized that gene augmentation of NRF2 or SIRT1 within RGCs could ameliorate pathologic features of experimental optic neuritis. Our data demonstrate distinct effects upon RGC survival and function following AAV2-mediated overexpression of NRF2 or SIRT1, suggesting these candidate factors promote neuroprotective mechanisms that may modify MS pathogenesis.

RGC-directed gene therapy with SIRT1 and NRF2 vectors revealed differential effects upon visual acuity during EAE. Visual acuity was not affected prior to EAE development with any of the vectors tested, suggesting vector delivery or transgene overexpression did not mediate unintended toxicity on retinal function. We observed a statistically significant decline in visual acuity beginning at day 21 postimmunization with all AAV2 and vehicle-treated animals subjected to EAE, whereas sham-induced cohorts presented robust responses throughout the experimental timeline. While Larabee et al. 13 reported that NRF2 knockout mice exhibit increased visual decline compared to wild-type cohorts during EAE, augmenting NRF2 activity with AAV2 gene transfer was unable to preserve visual acuity in the current study. Failure to reverse effects of knockout studies may be due to the limited number of RGCs (21%) infected with the AAV2 vectors in this study. However, interestingly, overexpression of SIRT1 mediated a trending increase in functional recovery beginning at day 28 postinduction compared to vehicle and AAV2-eGFP control groups subjected to EAE. This protective effect achieved statistical significance compared to the AAV2-eGFP control group at 35 and 42 days postinduction, which is remarkable given that only a subset of RGCs were transfected. This finding also correlates with prior investigations utilizing compounds that stimulate SIRT1 activity and demonstrate varying degrees of OKR preservation in the context of experimental optic neuritis as well as optic nerve crush. 19,26,27

RGC numbers were significantly reduced in all animal groups sensitized to EAE. However, we observed increased RGC numbers with SIRT1 and NRF2 gene augmentation compared to the AAV2-eGFP and vehicle treatment groups. SIRT1 gene transfer did not mediate a statistically significant increase in RGC numbers but only a positive trend in survival compared to EAE-induced controls. NRF2 gene transfer provided the most robust protective response with respect to total and regional RGC survival. This outcome is particularly interesting as NRF2 augmentation did not correlate with an improvement in retinal function as shown by OKR recordings. However, disparities between OKR and RGC survival have been previously documented in this model.27 Another explanation for this finding could be that NRF2 overexpression is simply supporting survival of the RGC cells bodies but unable to sustain function. This interpretation is consistent with findings by Xiong et al. 17 where NRF2 gene transfer-mediated transient yet significant preservation of RGCs following optic nerve crush without promoting axonal regeneration required for functional retention. Importantly, as indicated above, we only achieved approximately 21% RGC transduction with the AAV2 vector and previously described dose. Regarding the discrepancy between OKR preservation and RGC survival following AAV2-SIRT1 treatment, it is also possible that SIRT1 augmentation may influence the survival of ON direction-selective ganglion cells, which is the subset of cells that contribute to the OKR, but not mediate a statistically significant effect upon total RGC survival.46 Moreover, selection of a vector platform with enhanced capabilities for RGC transduction may provide a more potent means of cellular protection and functional preservation in this model. Recent developments utilizing rational design and in vivo selection have generated novel AAV capsids with improved potency and tropism for retinal cell types compared to naturally isolated serotypes such as AAV2.47-51 Further investigation into SIRT1 or NRF2-mediated neuroprotection in this model with an improved vector system is certainly warranted. The differential effects observed here also suggest a potential role for combined therapy with overexpression of both SIRT1 and NRF2. Due to limitations of the current transduction efficiency and the total volume that can be injected in the eye, coinjection of both vectors is not feasible, but future development of improved vector systems may allow investigation of a dual therapy.

While we did observe evidence of neuroprotection upon RGC function and viability, overexpression of neither NRF2 nor SIRT1 was able to suppress the inflammatory and demyelinating phenotype associated with optic neuritis. RGC-directed gene therapy did not influence immune recruitment to the optic nerve as shown by H&E histological analysis. This observation correlates with previous studies that examined small molecule-mediated neuroprotection during EAE. Specifically, pharmacologic activators of SIRT1, including resveratrol and related compounds, did not suppress inflammation in the spinal cord or optic nerve when administered at various doses in the same EAE model19 used in the current study. Interestingly, transgenic overexpression of human SIRT1 within neurons was able to reduce inflammation within spinal cord lesions. 24 Similar to the effects on immunomodulation, AAV2-mediated expression of NRF2 or SIRT1 did not alleviate optic nerve demyelination. While these approaches did not attenuate demyelination, other studies that examined antioxidant or mitochondrial-directed gene therapy strategies during EAE have shown preserved myelin in the optic nerve.34-38 However, discrepancies in animal models, EAE immunization protocols, and other components of study design limit a direct comparison with these reports. In addition, our findings with respect to inflammation and myelination may once again reflect the limited transduction efficiency of the AAV2 vector.

Collectively, this study demonstrates at least partial neuroprotective effects of NRF2 and SIRT1 gene augmentation in the context of experimental optic neuritis, and suggests an important role of these signals in MS pathogenesis. Moreover, it underscores the therapeutic potential of targeting conserved cell survival pathways or mechanisms to impede progression of complex neurodegenerative disease.

All publications cited in this specification are incorporated herein by reference, including McDougald et al, Investigative Ophthalmology & Visual Science March 2018, Vol. 59, 1212-1220. Similarly, the SEQ ID NOs which are referenced herein and which appear in the appended Sequence Listing are incorporated by reference, as is US Provisional Patent Application No. 62/488,989, filed Apr. 24, 2017. While the invention has been described with reference to particular embodiments, it will be appreciated that modifications can be made without departing from the spirit of the invention. Such modifications are intended to fall within the scope of the appended claims.

### (Sequence Listing Free Text)

The following information is provided for sequences containing free text under numeric identifier <223>.

| SEQ ID NO: | |
|---|---|
| (containing | |
| free text) | Free text under <223> |
| 5 | <223> constructed sequence |
| 6 | <223> constructed sequence |
| 7 | <223> constructed sequence |
| 8 | <223> constructed sequence |
| 9 | <223> constructed sequence |
| 10 | <223> constructed sequence |
| 11 | <223> constructed sequence |
| 12 | <223> constructed sequence |
| 13 | <223> constructed sequence |
| 14 | <223> constructed sequence |
| 15 | <223> constructed sequence |
| 16 | <223> constructed sequence |
| 17 | <223> constructed sequence |
| 18 | <223> constructed sequence |
| 19 | <223> constructed sequence |
| 20 | <223> constructed sequence |
| 21 | <223> constructed sequence |
| 22 | <223> constructed sequence |
| 23 | <223> constructed sequence |
| 24 | <223> constructed sequence |
| 25 | <223> constructed sequence |
| 26 | <223> constructed sequence |
| 27 | <223> constructed sequence |
| 28 | <223> constructed sequence |

### REFERENCES

1. Lassmann H, Horssen J V, Mahad D. Progressive multiple sclerosis: pathology and pathogenesis. Nat Rev Neurol. 2012; 8: 647-656.
2. Costello F, Coupland S, Hodge W, et al. Quantifying axonal loss after optic neuritis with optical coherence tomography. Ann Neurol. 2006; 59: 963-969.
3. Fisher J B, Jacobs D A, Markowitz C E, et al. Relation of visual function to retinal nerve fiber layer thickness in multiple sclerosis. Ophthalmol. 2006; 113: 324-332.
4. Trip S A, Schlottmann P G, Jones S J, et al. Retinal nerve fiber layer axonal loss and visual dysfunction in optic neuritis. Ann Neurol. 2005; 58: 383-391.
5. Beck R W, Cleary P A, Anderson M M Jr, et al. A randomized controlled trial of corticosteroids in the treatment of acute optic neuritis. The optic neuritis study group. N Engl J Med. 1992; 326: 581-588.
6. Lublin F D. Role of myelin antigens in murine relapsing experimental allergic encephalomyelitis. J Clin Lab Immunol. 1984; 13: 179-182.
7. Quinn T A, Dutt M, Shindler K S. Optic neuritis and retinal ganglion cell loss in a chronic murine model of multiple sclerosis. Front Neurol. 2011; 2.
8. Shindler K S, Guan Y, Ventura E, et al. Retinal ganglion cell loss induced by acute optic neuritis in a relapsing model of multiple sclerosis. Mult Scler. 2006; 12: 526-532.
9. Shindler K S, Ventura E, Dutt M, et al. Inflammatory demyelination induces axonal injury and retinal ganglion cell apoptosis in experimental optic neuritis. Exp Eye Res. 2008; 87: 208-213.
10. Dinkova-Kostova A T, Holtzclaw W D, Cole R N, et al. Direct evidence of sulfhydryl groups of KEAP1 are the sensors regulating induction of phase 2 enzymes that protect against carcinogens and oxidants. Proc Natl Acad Sci USA. 2002; 99: 11908-11913.
11. Johnson D A, Johnson J A. NRF2—a therapeutic target for the treatment of neurodegenerative diseases. Free Radic Biol Med. 2015; 88: 253-267.
12. Johnson D A, Amirahmadi S, Ward C, et al. The absence of the pro-antioxidant transcription factor NRF2 exacerbates experimental autoimmune encephalomyelitis. Toxicol Sci. 2010; 114: 237-246.
13. Larabee C M, Desai S, Agasing A, et al. Loss of Nrf2 exacerbates the visual deficits and optic neuritis elicited by experimental autoimmune encephalomyelitis. Mol Vis. 2016; 22: 1503-1513.
14. Li B, Cui W, Liu J, et al. Sulforaphane ameliorates the development of experimental autoimmune encephalomyelitis by antagonizing oxidative stress and Th17-related inflammation in mice. Exp Neurol. 2013; 250: 239-249.
15. Liang K J, Woodard K T, Weaver M A, et al. AAV-NRF2 promotes protection and recovery in animal models of oxidative stress. Mol Ther. 2017; 25: 765-779.
16. Linker R A, Lee D H, Ryan S, et al. Fumaric acid esters exert neuroprotective effects in neuroinflammation via activation of the Nrf2 antioxidant pathway. Brain. 2011; 134 (part 3): 678-692.
17. Xiong W, MacColl Garfinkel A E, Li Y, et al. NRF2 promotes neuronal survival in neurodegeneration and acute nerve damage. J Clin Invest. 2015; 125: 1433-1445.
18. Martin A, Tegla C A, Cudrici C D, et al. Role of SIRT1 in autoimmune demyelination and neurodegeneration. Immunol Res. 2015; 61: 187-197.
19. Fonseca-Kelly Z, Nassrallah M, Uribe J, et al. Resveratrol neuroprotection in a chronic mouse model of multiple sclerosis. Front Neurol. 2012; 3: 84.
20. Jeong H, Cohen D E, Cui L, et al. SIRT1 mediates neuroprotection from mutant huntingtin by activation of the TORC1 and CREB transcriptional pathway. Nat Med. 2011; 18: 159-165.
21. Khan R S, Dine K, Das Sarma J, et al. SIRT1 activating compounds reduce oxidative stress mediated neuronal loss in viral induced CNS demyelinating disease. Acta Neuropathol Commun. 2014; 2: 3.
22. Khan R S, Fonseca-Kelly Z, Callinan C, et al. SIRT1 activating compounds reduce oxidative stress and prevent cell death in neuronal cells. Front Cell Neurosci. 2012; 6: 63.
23. Kim D, Nguyen M D, Dobbin M M, et al. SIRT1 deacetylase protects against neurodegeneration in models for Alzheimer's disease and amyotrophic lateral sclerosis. EMBO J. 2007; 26: 3169-3179.
24. Nimmagadda V K, Bever C T, Vattikunta N R, et al. Overexpression of SIRT1 protein in neurons protects against experimental autoimmune encephalomyelitis through activation of multiple SIRT1 targets. J Immunol. 2013; 190: 4595-4607.
25. Shindler K S, Ventura E, Dutt M, et al. Oral resveratrol reduces neuronal damage in a model of multiple sclerosis. J Neuroophthalmol. 2010; 30: 328-339.
26. Shindler K S, Ventura E, Rex T S, et al. SIRT1 activation confers neuroprotection in experimental optic neuritis. Invest Ophthalmol Vis Sci. 2007; 48: 3602-3609.
27. Zuo L, Khan R S, Lee V, et al. SIRT1 promotes RGC survival and delays loss of function following optic nerve crush. Invest Ophthalmol Vis Sci. 2013; 54: 5097-5102.
28. Prusky G T, Alam N M, Beekman S, et al. Rapid quantification of adult and developing mouse spatial vision using a virtual optomotor system. Invest Ophthalmol Vis Sci. 2004; 45: 4611-4616.
29. Bennett J, Wellman J, Marshall K A, et al. Safety and durability of effect of contralateral-eye administration of AAV2 gene therapy in patients with childhood-onset blindness caused by RPE65 mutations: a follow-on phase 1 trial. Lancet. 2016; 388: 661-672.
30. Bennicelli J, Wright J F, Komaromy A, et al. Reversal of blindness in animal models of Leber congenital amaurosis using optimized AAV2-mediated gene transfer. Mol Ther. 2008; 16: 458-465.
31. Maguire A M, Simonelli F, Pierce E A, et al. Safety and efficacy of gene transfer for Leber's congenital amaurosis. N Engl J Med. 2008; 358: 2240-2248.
32. Pierce E A, Bennett J. The status of the RPE65 gene therapy trials: safety and efficacy. Cold Spring Harb Perspect Med. 2015; 5: a017285.
33. Dudus L, Anand V, Acland G M, et al. Persistent transgene product in retina, optic nerve and brain after intraocular injection of rAAV. Vision Res. 1999; 39: 2545-2553.
34. Qi X, Hauswirth W W, Guy J. Dual gene therapy with extracellular superoxide dismutase and catalase attenuates experimental optic neuritis. Mol Vis. 2007; 13: 1-11.
35. Qi X, Lewin As, Sun L, et al. Suppression of mitochondrial oxidative stress provides long-term neuroprotection in experimental optic neuritis. Invest Ophthalmol Vis Sci. 2007; 48: 681-691.
36. Talla V, Koilkonda R, Porciatti V, et al. Complex I subunit gene therapy with NDUFA6 ameliorates neurodegeneration in EAE. Invest Ophthalmol Vis Sci. 2015; 56: 1129-1140.
37. Talla V, Porciatti V, Chiodo V, et al. Gene therapy with mitochondrial heat shock protein 70 suppresses visual loss and optic atrophy in experimental autoimmune encephalomyelitis. Invest Ophthalmol Vis Sci. 2014; 55: 5214-5226.
38. Talla V, Yu H, Chou T H, et al. NADH-dehydrogenase type-2 suppresses irreversible visual loss and neurodegeneration in the EAE animal model of M S. Mol Ther. 2013; 21: 1876-1888.
39. Itoh K, Wakabayashi N, Katoh Y, et al. Keap1 regulates both cytoplasmic-nuclear shuttling and degradation of NRF2 in response to electrophiles. Genes Cells. 2003; 8: 379-391.
40. McMahon M, Itoh K, Yamamoto M, et al. Keap1-dependent proteasomal degradation of transcription factor NRF2 contributes to negative regulation of antioxidant response element-driven gene expression. J Biol Chem. 2003; 278: 21592-21600.
41. Luo J, Nikolaev A Y, Imai S, et al. Negative control of p53 by Sir2 alpha promotes cell survival under stress. Cell. 2001; 107: 137-148.
42. Nemoto S, Fergusson M M, Finkel T. SIRT1 functionally interacts with the metabolic regulator and transcriptional coactivator PGC-1α. J Biol Chem. 2005; 280: 16456-16460.
43. Ding Y W, Zhao G J, Li X L, et al. SIRT1 exerts protective effects against paraquat-induced injury in mouse type II alveolar epithelial cells by deacetylating NRF2 in vitro. Int J Mol Med. 2016; 37: 1049-1058.
44. Xia X, Qu B, Li Y M, et al. NFAT5 protects astrocytes against oxygen-glucose-serum deprivation/restoration damage via the SIRT1/Nrf2 pathway. J Mol Neurosci. 2017; 61: 96-104.
45. Zhang P, Li Y, Du Y, et al. Resveratrol ameliorated vascular calcification by regulating Sirt-1 and Nrf2. Transplant Proc. 2016; 48: 3378-3386.
46. Sugita Y, Miura K, Araki F, et al. Contributions of retinal direction-selective ganglion cells to optokinetic responses in mice. Eur J Neurosci. 2013; 38: 2823-2831.
47. Choudhury S R, Fitzpatrick Z, Harris A F, et al. In vivo selection yields AAV-B1 capsid for central nervous system and muscle gene therapy. Mol Ther. 2016; 1247-1257.
48. Cronin T, Vandenberghe L H, Hantz P, et al. Efficient transduction and optogenetic stimulation of retinal bipolar cells by a synthetic adeno-associated virus capsid and promoter. EMBO Mol Med. 2014; 6: 1175-1190.
49. Dalkara D, Byrne L C, Klimczak R R, et al. In vivo-directed evolution of a new adeno-associated virus for therapeutic outer retinal gene delivery from the vitreous. Sci Transl Med. 2013; 5: 189.
50. Deverman B E, Pravdo P L, Simpson B P, et al. Cre-dependent selection yields AAV variants for widespread gene transfer to the adult brain. Nat Biotechnol. 2016; 34: 204-209.
51. Zinn E, Pacouret S, Khaychuk V, et al. In silico reconstruction of the viral evolutionary lineage yields a potent gene therapy vector. Cell Rep. 2015; 12: 1056-1068.

1. A recombinant adeno-associated virus (rAAV), said rAAV comprising an AAV capsid, and a vector genome packaged therein, said vector genome comprising:
(a) an AAV 5′ inverted terminal repeat (ITR) sequence;
(b) a promoter;
(c) a coding sequence encoding a human NRF2; and
(d) an AAV 3′ ITR.

2. The rAAV according to claim 1, wherein the coding sequence of (c) is SEQ ID NO: 4 or SEQ ID NO: 7.

3. The rAAV according to claim 1, wherein the vector genome comprises nt 1253 to nt5245 of SEQ ID NO: 5, or nt 1253 to nt 5359 of SEQ ID NO: 10, or 1253 to nt 5359 of SEQ ID NO: 8, or nt 1253 to nt 5359 of SEQ ID NO: 21, or nt 1253 to nt 5425 of SEQ ID NO: 22, or nt 1253 to nt 5351 of SEQ ID NO: 23, or nt 1253 to nt 5378 of SEQ ID NO: 24, or nt 1253 to nt 5253 of SEQ ID NO: 25, or nt 1253 to nt 5280 of SEQ ID NO: 26, or nt 1253 to nt 5253 of SEQ ID NO: 27, or nt 1253 to nt 5280 of SEQ ID NO: 28.

4. The rAAV according to claim 1,
wherein the rAAV capsid is an AAV2 capsid or variant thereof, AAV7m8 or variant thereof, an AAV8 capsid, an AAV6 capsid or variant thereof, an AAV9 capsid or variant thereof, an AAV7 capsid, or variant thereof, an AAV5 capsid, or variant thereof, an AAV1 capsid or variant thereof, an AAV3 capsid or variant thereof, or an AAV4 capsid or variant thereof.

5. The rAAV according to claim 1, wherein the promoter is a cytomegalovirus (CMV) promoter or a hybrid promoter comprising a CMV promoter sequence and a chicken beta actin (CBA) promoter sequence.

6.-7. (canceled)

8. The rAAV according to claim 1, wherein the AAV 5′ ITR and/or AAV3′ ITR is from AAV2.

9.-12. (canceled)

13. A composition comprising the rAAV of claim 1 and a pharmaceutically acceptable carrier or excipient suitable for delivery to the eye.

14.-16. (canceled)

17. A method of treating or preventing optic neuropathy in a subject in need thereof with a rAAV according to claim 1.

18. The method according to claim 17, wherein the rAAV is delivered about 1×10⁹ to about 1×10¹³ vector genomes per eye (vg/eye) in an aqueous suspension.

19.-25. (canceled)

26. A recombinant adeno-associated virus (rAAV), said rAAV comprising an AAV capsid, and a vector genome packaged therein, said vector genome comprising:
(a) an AAV 5′ inverted terminal repeat (ITR) sequence;
(b) a promoter;
(c) a coding sequence encoding a human SIRT1; and
(d) an AAV 3′ ITR.

27. The rAAV according to claim 26, wherein the coding sequence of (c) is SEQ ID NO: 2 or SEQ ID NO: 12.

28. The rAAV according to claim 26, wherein the vector genome comprises nt 1253 to nt 5854 of SEQ ID NO: 6, or nt 1253 to nt5788 of SEQ ID NO: 9, or nt 1253 to nt 5777 of SEQ ID NO: 13, or nt 1253 to nt 5854 of SEQ ID NO: 14, or nt 1253 to nt 5792 of SEQ ID NO: 15, or nt 1253 to nt 5819 of SEQ ID NO: 16, or nt 1253 to nt 5777 of SEQ ID NO: 17, or nt 1253 to nt 5706 of SEQ ID NO: 18, or nt 1253 to nt 5694 of SEQ ID NO: 19, or nt 1253 to nt 5721 of SEQ ID NO: 20.

29. The rAAV according to claim 26, wherein the rAAV capsid is an AAV2 capsid or variant thereof, AAV7m8 or variant thereof, an AAV8 capsid, an AAV6 capsid or variant thereof, an AAV9 capsid or variant thereof, an AAV7 capsid, or variant thereof, an AAV5 capsid, or variant thereof, an AAV1 capsid or variant thereof, an AAV3 capsid or variant thereof, or an AAV4 capsid or variant thereof.

30. The rAAV according to claim 26, wherein the promoter is a cytomegalovirus (CMV) promoter or a hybrid promoter comprising a CMV promoter sequence and a chicken beta actin (CBA) promoter sequence.

31.-32. (canceled)

33. The rAAV according to claim 26, wherein the AAV 5′ ITR and/or AAV3′ ITR is from AAV2.

34.-37. (canceled)

38. A composition comprising the rAAV of claim 26 and a pharmaceutically acceptable carrier or excipient suitable for delivery to the eye.

39.-41. (canceled)

42. A method of treating or preventing optic neuropathy in a subject in need thereof with a rAAV according to any of claim 26.

43. The method according to claim 42, wherein the rAAV is delivered about 1×10⁹ to about 1×10¹³ vector genomes per eye (vg/eye) in an aqueous suspension.

44.-50. (canceled)

51. A method of preserving retinal ganglion cell (RGC) function in a subject, comprising administering the rAAV of claim 1.

52. A method of preserving retinal ganglion cell (RGC) function in a subject, comprising administering the rAAV of claim 26.


SEQUENCE LISTING (28 sequences)

SEQ ID NO: 1 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 2 - DNA, 2241 bp, Homo sapiens
  source 1..2241: mol_type = other DNA
atggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatca

SEQ ID NO: 3 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 4 - DNA, 1815 bp, Homo sapiens
  source 1..1815: mol_type = other DNA
atgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaac

SEQ ID NO: 5 - DNA, 12025 bp, synthetic construct
  misc_feature 1..12025: note = constructed sequence
  source 1..12025: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaacgactacaaagaccatgacggtgattataaagatcatgacatcgattacaaggatgacgatgacaagtgaggccgcatagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 6 - DNA, 12454 bp, synthetic construct
  misc_feature 1..12454: note = constructed sequence
  source 1..12454: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcagactacaaagaccatgacggtgattataaagatcatgacatcgattacaaggatgacgatgacaagtgatgaggccgcatagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 7 - DNA, 1815 bp, synthetic construct
  misc_feature 1..1815: note = constructed sequence
  source 1..1815: mol_type = other DNA
atgatggacctcgaactgccgccgcctggcctcccaagccaacaggatatggacctgattgacatcctgtggcggcaggacattgatctgggtgtcagccgcgaggtgttcgatttctcgcaacgccggaaggaatacgaactcgagaagcagaagaagctcgagaaagagcggcaggaacagctccagaaggaacaggaaaaggccttcttcgcacaacttcagctggacgaggaaaccggcgaattcctgcctattcaaccagcccagcacatccagagcgaaacctccggcagcgccaactattcccaagtggctcacatcccgaagtccgacgccctgtactttgacgattgtatgcagctgctggcacagaccttccccttcgtcgatgataacgaggtgtcctccgcgacgtttcagtcgctggtccccgacatccccggtcatatcgagagccctgtgttcatcgccaccaaccaggctcagtcccccgaaacctcagtggcacaagtggcgccggtggacttggacggcatgcagcaagacatcgaacaagtctgggaggagcttctgtccatccccgagctgcaatgcctcaacatcgagaatgacaagctcgtggagactactatggtcccgtccccggaagctaagctgaccgaggtcgacaactaccatttctactcctcaatcccctccatggaaaaggaagtcggaaactgctcgcctcatttcctcaacgccttcgaggactccttctcgtcaattctgtccactgaggaccccaaccagctgaccgtcaattccttgaactcggatgccactgtgaacaccgacttcggcgacgaattctacagcgcgttcatcgccgaaccgagcatctcgaactccatgccctcgcccgccaccttgtcacattccctgtctgagctgctgaacgggccgattgacgtgtcagacctgagcctgtgtaaagccttcaaccagaatcacccggagtcgactgccgaattcaacgactcggactccgggatctcactgaacactagccctagcgtggcctcgcccgaacactccgtggagtccagctcctatggcgatactcttctgggtctgtccgactccgaagtggaagaactggactctgcccccggaagcgtgaaacagaacggacctaagaccccagtgcactcctccggggatatggtgcagccgttgtcaccgagccaggggcaatccacccacgtgcatgacgctcagtgcgagaacacccccgagaaagaactcccagtgtcccccggacaccgaaagaccccgtttaccaaggacaagcactcctcacggctggaagcacaccttactcgggatgaactcagagccaaggccctccacattcctttccccgtggagaagattatcaatctccctgtggtggatttcaacgagatgatgagcaaggaacagttcaacgaagcgcagctggcgctgatcagggacatcaggcgcagaggaaagaacaaagtggccgcccaaaactgccggaagagaaagctcgaaaacatcgtggagctcgaacaggacttggaccacctgaaggatgaaaaagaaaagctgctgaaggagaagggagagaacgacaagtccctccatctgctgaagaagcagctgagcacactgtacctcgaagtgttttccatgctgcgcgatgaggatggaaagccgtactccccgtccgaatactcgctgcaacagacgcgcgacggaaacgtgttcctcgtgccaaagtccaagaagcctgacgtgaagaagaac

SEQ ID NO: 8 - DNA, 11951 bp, synthetic construct
  misc_feature 1..11951: note = constructed sequence
  source 1..11951: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatgatggacctcgaactgccgccgcctggcctcccaagccaacaggatatggacctgattgacatcctgtggcggcaggacattgatctgggtgtcagccgcgaggtgttcgatttctcgcaacgccggaaggaatacgaactcgagaagcagaagaagctcgagaaagagcggcaggaacagctccagaaggaacaggaaaaggccttcttcgcacaacttcagctggacgaggaaaccggcgaattcctgcctattcaaccagcccagcacatccagagcgaaacctccggcagcgccaactattcccaagtggctcacatcccgaagtccgacgccctgtactttgacgattgtatgcagctgctggcacagaccttccccttcgtcgatgataacgaggtgtcctccgcgacgtttcagtcgctggtccccgacatccccggtcatatcgagagccctgtgttcatcgccaccaaccaggctcagtcccccgaaacctcagtggcacaagtggcgccggtggacttggacggcatgcagcaagacatcgaacaagtctgggaggagcttctgtccatccccgagctgcaatgcctcaacatcgagaatgacaagctcgtggagactactatggtcccgtccccggaagctaagctgaccgaggtcgacaactaccatttctactcctcaatcccctccatggaaaaggaagtcggaaactgctcgcctcatttcctcaacgccttcgaggactccttctcgtcaattctgtccactgaggaccccaaccagctgaccgtcaattccttgaactcggatgccactgtgaacaccgacttcggcgacgaattctacagcgcgttcatcgccgaaccgagcatctcgaactccatgccctcgcccgccaccttgtcacattccctgtctgagctgctgaacgggccgattgacgtgtcagacctgagcctgtgtaaagccttcaaccagaatcacccggagtcgactgccgaattcaacgactcggactccgggatctcactgaacactagccctagcgtggcctcgcccgaacactccgtggagtccagctcctatggcgatactcttctgggtctgtccgactccgaagtggaagaactggactctgcccccggaagcgtgaaacagaacggacctaagaccccagtgcactcctccggggatatggtgcagccgttgtcaccgagccaggggcaatccacccacgtgcatgacgctcagtgcgagaacacccccgagaaagaactcccagtgtcccccggacaccgaaagaccccgtttaccaaggacaagcactcctcacggctggaagcacaccttactcgggatgaactcagagccaaggccctccacattcctttccccgtggagaagattatcaatctccctgtggtggatttcaacgagatgatgagcaaggaacagttcaacgaagcgcagctggcgctgatcagggacatcaggcgcagaggaaagaacaaagtggccgcccaaaactgccggaagagaaagctcgaaaacatcgtggagctcgaacaggacttggaccacctgaaggatgaaaaagaaaagctgctgaaggagaagggagagaacgacaagtccctccatctgctgaagaagcagctgagcacactgtacctcgaagtgttttccatgctgcgcgatgaggatggaaagccgtactccccgtccgaatactcgctgcaacagacgcgcgacggaaacgtgttcctcgtgccaaagtccaagaagcctgacgtgaagaagaactgaagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 9 - DNA, 12388 bp, synthetic construct
  misc_feature 1..12388: note = constructed sequence
  source 1..12388: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatgatgaggccgcatagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 10 - DNA, 11959 bp, synthetic construct
  misc_feature 1..11959: note = constructed sequence
  source 1..11959: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactgaggccgcatagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 11 - DNA, 12392 bp, synthetic construct
  misc_feature 1..12392: note = constructed sequence
  source 1..12392: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgcgccgccaccatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatgagcggccgcatagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 12 - DNA, 2241 bp, synthetic construct
  misc_feature 1..2241: note = constructed sequence
  source 1..2241: mol_type = other DNA
atggccgacgaagccgcactcgcgctgcaaccagggggttccccgtctgcagcaggcgccgatagagaagcagccagctcgccagcaggagaaccgctccggaagcgacccaggagagatggcccaggcctggaacgctcaccgggtgaaccaggaggtgccgctcctgaacgggaagtgcccgcagctgctaggggctgtccaggagcagccgcagcagcactctggcgcgaagccgaagccgaggctgctgcagccggaggagaacaagaagcccaagcgaccgctgcagcaggagagggcgacaacggacccggcctgcaaggaccgtcccgcgaacctccgctggccgacaatctgtacgacgaggacgatgatgatgagggcgaagaagaagaggaagccgccgctgctgctattggatacagagacaaccttctcttcggcgacgagatcatcaccaatggattccactcctgtgaatctgacgaagaagatcgggcctcccatgctagcagctctgactggacgccacggccaaggatcggtccgtacaccttcgtgcagcagcacctgatgatcggcacagatccccgcaccattctgaaggatctgctgcccgagactatcccaccccctgagctggacgacatgactctgtggcagattgtgatcaacatcctttccgagcccccaaagcggaagaagcgcaaggatattaacaccatcgaggacgccgtgaagcttctgcaggagtgcaaaaagatcatcgtgctgactggtgcaggggtgtccgtgtcctgcggcattccggatttcagaagccgcgacggaatctacgccagactcgcggtcgactttcccgatctgcctgaccctcaggccatgttcgatattgaatacttccgcaaggacccgaggccgttctttaagttcgccaaggagatctaccctgggcagttccaaccctccctctgccataagttcattgcgctgagcgataaggaaggaaagctgctgcggaactacacccagaacatcgacactcttgagcaagtggcgggtatccagagaatcatccaatgccacggctccttcgccactgcctcctgcctgatctgcaagtacaaggtcgattgtgaagccgtcaggggcgatatcttcaaccaagtggtcccgcgatgcccaagatgcccggcggatgaacccttggccatcatgaagcctgaaatcgtgttcttcggggaaaacctccccgaacagtttcaccgcgccatgaagtacgacaaggacgaagtggatctcctgattgtgattggttccagcttgaaagtccggccagtggccttgatcccctcctcgattccacacgaagtgcctcaaatccttattaaccgggaaccactgccgcatcttcacttcgatgtggaactgctgggagactgcgacgtgattattaacgaactgtgtcaccgcttgggtggcgaatacgccaagctgtgctgcaaccctgtgaaactgtcggagatcaccgaaaagccgcccagaactcaaaaagagctggcctacctgtccgagctccctccgactccgctgcatgtgtccgaggattcgtcgtccccggaaaggacttcgcctccggactcctccgtgatcgtgaccctgctcgaccaagccgccaagtcgaacgacgatctggacgtgtcggaatccaagggctgcatggaagaaaagcctcaagaggtgcagacttcacggaacgtggagtccatcgccgaacagatggaaaacccggacctgaagaacgtgggaagcagcactggcgaaaagaacgagagaacctccgtggccggtactgtccggaagtgctggccgaacagggtggccaaggaacagatatcccggagacttgacggcaaccagtacctcttcctcccccctaaccgctatatttttcacggggccgaggtgtacagcgactccgaggacgatgtgctgtcatcctcatcatgcggttccaattccgactccggaacctgtcagagcccctccctggaggaacctatggaggacgaatccgagatcgaggaattctacaacggtctggaggacgagcctgatgtcccggaacgcgctggaggagctggcttcggaaccgacggggacgaccaggaagccatcaacgaggccatctcggtgaagcaggaagtgaccgatatgaactacccgtccaacaagtcc

SEQ ID NO: 13 - DNA, 12377 bp, synthetic construct
  misc_feature 1..12377: note = constructed sequence
  source 1..12377: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatgaagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 14 - DNA, 12454 bp, synthetic construct; identical to SEQ ID NO: 6
  misc_feature 1..12454: note = constructed sequence
  source 1..12454: mol_type = other DNA

SEQ ID NO: 15 - DNA, 12392 bp, synthetic construct
  misc_feature 1..12392: note = constructed sequence
  source 1..12392: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgcgccgccaccatggccgacgaagccgcactcgcgctgcaaccagggggttccccgtctgcagcaggcgccgatagagaagcagccagctcgccagcaggagaaccgctccggaagcgacccaggagagatggcccaggcctggaacgctcaccgggtgaaccaggaggtgccgctcctgaacgggaagtgcccgcagctgctaggggctgtccaggagcagccgcagcagcactctggcgcgaagccgaagccgaggctgctgcagccggaggagaacaagaagcccaagcgaccgctgcagcaggagagggcgacaacggacccggcctgcaaggaccgtcccgcgaacctccgctggccgacaatctgtacgacgaggacgatgatgatgagggcgaagaagaagaggaagccgccgctgctgctattggatacagagacaaccttctcttcggcgacgagatcatcaccaatggattccactcctgtgaatctgacgaagaagatcgggcctcccatgctagcagctctgactggacgccacggccaaggatcggtccgtacaccttcgtgcagcagcacctgatgatcggcacagatccccgcaccattctgaaggatctgctgcccgagactatcccaccccctgagctggacgacatgactctgtggcagattgtgatcaacatcctttccgagcccccaaagcggaagaagcgcaaggatattaacaccatcgaggacgccgtgaagcttctgcaggagtgcaaaaagatcatcgtgctgactggtgcaggggtgtccgtgtcctgcggcattccggatttcagaagccgcgacggaatctacgccagactcgcggtcgactttcccgatctgcctgaccctcaggccatgttcgatattgaatacttccgcaaggacccgaggccgttctttaagttcgccaaggagatctaccctgggcagttccaaccctccctctgccataagttcattgcgctgagcgataaggaaggaaagctgctgcggaactacacccagaacatcgacactcttgagcaagtggcgggtatccagagaatcatccaatgccacggctccttcgccactgcctcctgcctgatctgcaagtacaaggtcgattgtgaagccgtcaggggcgatatcttcaaccaagtggtcccgcgatgcccaagatgcccggcggatgaacccttggccatcatgaagcctgaaatcgtgttcttcggggaaaacctccccgaacagtttcaccgcgccatgaagtacgacaaggacgaagtggatctcctgattgtgattggttccagcttgaaagtccggccagtggccttgatcccctcctcgattccacacgaagtgcctcaaatccttattaaccgggaaccactgccgcatcttcacttcgatgtggaactgctgggagactgcgacgtgattattaacgaactgtgtcaccgcttgggtggcgaatacgccaagctgtgctgcaaccctgtgaaactgtcggagatcaccgaaaagccgcccagaactcaaaaagagctggcctacctgtccgagctccctccgactccgctgcatgtgtccgaggattcgtcgtccccggaaaggacttcgcctccggactcctccgtgatcgtgaccctgctcgaccaagccgccaagtcgaacgacgatctggacgtgtcggaatccaagggctgcatggaagaaaagcctcaagaggtgcagacttcacggaacgtggagtccatcgccgaacagatggaaaacccggacctgaagaacgtgggaagcagcactggcgaaaagaacgagagaacctccgtggccggtactgtccggaagtgctggccgaacagggtggccaaggaacagatatcccggagacttgacggcaaccagtacctcttcctcccccctaaccgctatatttttcacggggccgaggtgtacagcgactccgaggacgatgtgctgtcatcctcatcatgcggttccaattccgactccggaacctgtcagagcccctccctggaggaacctatggaggacgaatccgagatcgaggaattctacaacggtctggaggacgagcctgatgtcccggaacgcgctggaggagctggcttcggaaccgacggggacgaccaggaagccatcaacgaggccatctcggtgaagcaggaagtgaccgatatgaactacccgtccaacaagtcctgagcggccgcatagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 16 - DNA, 12419 bp, synthetic construct
  misc_feature 1..12419: note = constructed sequence
  source 1..12419: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgcgccgccaccatggccgacgaagccgcactcgcgctgcaaccagggggttccccgtctgcagcaggcgccgatagagaagcagccagctcgccagcaggagaaccgctccggaagcgacccaggagagatggcccaggcctggaacgctcaccgggtgaaccaggaggtgccgctcctgaacgggaagtgcccgcagctgctaggggctgtccaggagcagccgcagcagcactctggcgcgaagccgaagccgaggctgctgcagccggaggagaacaagaagcccaagcgaccgctgcagcaggagagggcgacaacggacccggcctgcaaggaccgtcccgcgaacctccgctggccgacaatctgtacgacgaggacgatgatgatgagggcgaagaagaagaggaagccgccgctgctgctattggatacagagacaaccttctcttcggcgacgagatcatcaccaatggattccactcctgtgaatctgacgaagaagatcgggcctcccatgctagcagctctgactggacgccacggccaaggatcggtccgtacaccttcgtgcagcagcacctgatgatcggcacagatccccgcaccattctgaaggatctgctgcccgagactatcccaccccctgagctggacgacatgactctgtggcagattgtgatcaacatcctttccgagcccccaaagcggaagaagcgcaaggatattaacaccatcgaggacgccgtgaagcttctgcaggagtgcaaaaagatcatcgtgctgactggtgcaggggtgtccgtgtcctgcggcattccggatttcagaagccgcgacggaatctacgccagactcgcggtcgactttcccgatctgcctgaccctcaggccatgttcgatattgaatacttccgcaaggacccgaggccgttctttaagttcgccaaggagatctaccctgggcagttccaaccctccctctgccataagttcattgcgctgagcgataaggaaggaaagctgctgcggaactacacccagaacatcgacactcttgagcaagtggcgggtatccagagaatcatccaatgccacggctccttcgccactgcctcctgcctgatctgcaagtacaaggtcgattgtgaagccgtcaggggcgatatcttcaaccaagtggtcccgcgatgcccaagatgcccggcggatgaacccttggccatcatgaagcctgaaatcgtgttcttcggggaaaacctccccgaacagtttcaccgcgccatgaagtacgacaaggacgaagtggatctcctgattgtgattggttccagcttgaaagtccggccagtggccttgatcccctcctcgattccacacgaagtgcctcaaatccttattaaccgggaaccactgccgcatcttcacttcgatgtggaactgctgggagactgcgacgtgattattaacgaactgtgtcaccgcttgggtggcgaatacgccaagctgtgctgcaaccctgtgaaactgtcggagatcaccgaaaagccgcccagaactcaaaaagagctggcctacctgtccgagctccctccgactccgctgcatgtgtccgaggattcgtcgtccccggaaaggacttcgcctccggactcctccgtgatcgtgaccctgctcgaccaagccgccaagtcgaacgacgatctggacgtgtcggaatccaagggctgcatggaagaaaagcctcaagaggtgcagacttcacggaacgtggagtccatcgccgaacagatggaaaacccggacctgaagaacgtgggaagcagcactggcgaaaagaacgagagaacctccgtggccggtactgtccggaagtgctggccgaacagggtggccaaggaacagatatcccggagacttgacggcaaccagtacctcttcctcccccctaaccgctatatttttcacggggccgaggtgtacagcgactccgaggacgatgtgctgtcatcctcatcatgcggttccaattccgactccggaacctgtcagagcccctccctggaggaacctatggaggacgaatccgagatcgaggaattctacaacggtctggaggacgagcctgatgtcccggaacgcgctggaggagctggcttcggaaccgacggggacgaccaggaagccatcaacgaggccatctcggtgaagcaggaagtgaccgatatgaactacccgtccaacaagtcctacccatacgatgttccagattacgcttgagcggccgcatagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 17 - DNA, 12377 bp, synthetic construct; identical to SEQ ID NO: 13
  misc_feature 1..12377: note = constructed sequence
  source 1..12377: mol_type = other DNA

SEQ ID NO: 18 - DNA, 12306 bp, synthetic construct
  misc_feature 1..12306: note = constructed sequence
  source 1..12306: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagccacccacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaaggcaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacaccctgtacactagcggccgccaccatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatacccatacgatgttccagattacgcttgaagtactgcggatcctgcaggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 19 - DNA, 12294 bp, synthetic construct
  misc_feature 1..12294: note = constructed sequence
  source 1..12294: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagccacccacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaaggcaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacaccctgtacactagcggccgcgccgccaccatggccgacgaagccgcactcgcgctgcaaccagggggttccccgtctgcagcaggcgccgatagagaagcagccagctcgccagcaggagaaccgctccggaagcgacccaggagagatggcccaggcctggaacgctcaccgggtgaaccaggaggtgccgctcctgaacgggaagtgcccgcagctgctaggggctgtccaggagcagccgcagcagcactctggcgcgaagccgaagccgaggctgctgcagccggaggagaacaagaagcccaagcgaccgctgcagcaggagagggcgacaacggacccggcctgcaaggaccgtcccgcgaacctccgctggccgacaatctgtacgacgaggacgatgatgatgagggcgaagaagaagaggaagccgccgctgctgctattggatacagagacaaccttctcttcggcgacgagatcatcaccaatggattccactcctgtgaatctgacgaagaagatcgggcctcccatgctagcagctctgactggacgccacggccaaggatcggtccgtacaccttcgtgcagcagcacctgatgatcggcacagatccccgcaccattctgaaggatctgctgcccgagactatcccaccccctgagctggacgacatgactctgtggcagattgtgatcaacatcctttccgagcccccaaagcggaagaagcgcaaggatattaacaccatcgaggacgccgtgaagcttctgcaggagtgcaaaaagatcatcgtgctgactggtgcaggggtgtccgtgtcctgcggcattccggatttcagaagccgcgacggaatctacgccagactcgcggtcgactttcccgatctgcctgaccctcaggccatgttcgatattgaatacttccgcaaggacccgaggccgttctttaagttcgccaaggagatctaccctgggcagttccaaccctccctctgccataagttcattgcgctgagcgataaggaaggaaagctgctgcggaactacacccagaacatcgacactcttgagcaagtggcgggtatccagagaatcatccaatgccacggctccttcgccactgcctcctgcctgatctgcaagtacaaggtcgattgtgaagccgtcaggggcgatatcttcaaccaagtggtcccgcgatgcccaagatgcccggcggatgaacccttggccatcatgaagcctgaaatcgtgttcttcggggaaaacctccccgaacagtttcaccgcgccatgaagtacgacaaggacgaagtggatctcctgattgtgattggttccagcttgaaagtccggccagtggccttgatcccctcctcgattccacacgaagtgcctcaaatccttattaaccgggaaccactgccgcatcttcacttcgatgtggaactgctgggagactgcgacgtgattattaacgaactgtgtcaccgcttgggtggcgaatacgccaagctgtgctgcaaccctgtgaaactgtcggagatcaccgaaaagccgcccagaactcaaaaagagctggcctacctgtccgagctccctccgactccgctgcatgtgtccgaggattcgtcgtccccggaaaggacttcgcctccggactcctccgtgatcgtgaccctgctcgaccaagccgccaagtcgaacgacgatctggacgtgtcggaatccaagggctgcatggaagaaaagcctcaagaggtgcagacttcacggaacgtggagtccatcgccgaacagatggaaaacccggacctgaagaacgtgggaagcagcactggcgaaaagaacgagagaacctccgtggccggtactgtccggaagtgctggccgaacagggtggccaaggaacagatatcccggagacttgacggcaaccagtacctcttcctcccccctaaccgctatatttttcacggggccgaggtgtacagcgactccgaggacgatgtgctgtcatcctcatcatgcggttccaattccgactccggaacctgtcagagcccctccctggaggaacctatggaggacgaatccgagatcgaggaattctacaacggtctggaggacgagcctgatgtcccggaacgcgctggaggagctggcttcggaaccgacggggacgaccaggaagccatcaacgaggccatctcggtgaagcaggaagtgaccgatatgaactacccgtccaacaagtcctgagcggccgcatagtactgcggatcctgcaggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 20 - DNA, 12321 bp, synthetic construct
  misc_feature 1..12321: note = constructed sequence
  source 1..12321: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagccacccacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaaggcaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacaccctgtacactagcggccgcgccgccaccatggccgacgaagccgcactcgcgctgcaaccagggggttccccgtctgcagcaggcgccgatagagaagcagccagctcgccagcaggagaaccgctccggaagcgacccaggagagatggcccaggcctggaacgctcaccgggtgaaccaggaggtgccgctcctgaacgggaagtgcccgcagctgctaggggctgtccaggagcagccgcagcagcactctggcgcgaagccgaagccgaggctgctgcagccggaggagaacaagaagcccaagcgaccgctgcagcaggagagggcgacaacggacccggcctgcaaggaccgtcccgcgaacctccgctggccgacaatctgtacgacgaggacgatgatgatgagggcgaagaagaagaggaagccgccgctgctgctattggatacagagacaaccttctcttcggcgacgagatcatcaccaatggattccactcctgtgaatctgacgaagaagatcgggcctcccatgctagcagctctgactggacgccacggccaaggatcggtccgtacaccttcgtgcagcagcacctgatgatcggcacagatccccgcaccattctgaaggatctgctgcccgagactatcccaccccctgagctggacgacatgactctgtggcagattgtgatcaacatcctttccgagcccccaaagcggaagaagcgcaaggatattaacaccatcgaggacgccgtgaagcttctgcaggagtgcaaaaagatcatcgtgctgactggtgcaggggtgtccgtgtcctgcggcattccggatttcagaagccgcgacggaatctacgccagactcgcggtcgactttcccgatctgcctgaccctcaggccatgttcgatattgaatacttccgcaaggacccgaggccgttctttaagttcgccaaggagatctaccctgggcagttccaaccctccctctgccataagttcattgcgctgagcgataaggaaggaaagctgctgcggaactacacccagaacatcgacactcttgagcaagtggcgggtatccagagaatcatccaatgccacggctccttcgccactgcctcctgcctgatctgcaagtacaaggtcgattgtgaagccgtcaggggcgatatcttcaaccaagtggtcccgcgatgcccaagatgcccggcggatgaacccttggccatcatgaagcctgaaatcgtgttcttcggggaaaacctccccgaacagtttcaccgcgccatgaagtacgacaaggacgaagtggatctcctgattgtgattggttccagcttgaaagtccggccagtggccttgatcccctcctcgattccacacgaagtgcctcaaatccttattaaccgggaaccactgccgcatcttcacttcgatgtggaactgctgggagactgcgacgtgattattaacgaactgtgtcaccgcttgggtggcgaatacgccaagctgtgctgcaaccctgtgaaactgtcggagatcaccgaaaagccgcccagaactcaaaaagagctggcctacctgtccgagctccctccgactccgctgcatgtgtccgaggattcgtcgtccccggaaaggacttcgcctccggactcctccgtgatcgtgaccctgctcgaccaagccgccaagtcgaacgacgatctggacgtgtcggaatccaagggctgcatggaagaaaagcctcaagaggtgcagacttcacggaacgtggagtccatcgccgaacagatggaaaacccggacctgaagaacgtgggaagcagcactggcgaaaagaacgagagaacctccgtggccggtactgtccggaagtgctggccgaacagggtggccaaggaacagatatcccggagacttgacggcaaccagtacctcttcctcccccctaaccgctatatttttcacggggccgaggtgtacagcgactccgaggacgatgtgctgtcatcctcatcatgcggttccaattccgactccggaacctgtcagagcccctccctggaggaacctatggaggacgaatccgagatcgaggaattctacaacggtctggaggacgagcctgatgtcccggaacgcgctggaggagctggcttcggaaccgacggggacgaccaggaagccatcaacgaggccatctcggtgaagcaggaagtgaccgatatgaactacccgtccaacaagtcctacccatacgatgttccagattacgcttgagcggccgcatagtactgcggatcctgcaggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 21 - DNA, 11959 bp, synthetic construct; identical to SEQ ID NO: 10
  misc_feature 1..11959: note = constructed sequence
  source 1..11959: mol_type = other DNA

SEQ ID NO: 22 - DNA, 12025 bp, synthetic construct; identical to SEQ ID NO: 5
  misc_feature 1..12025: note = constructed sequence
  source 1..12025: mol_type = other DNA

SEQ ID NO: 23 - DNA, 11951 bp, synthetic construct; identical to SEQ ID NO: 8
  misc_feature 1..11951: note = constructed sequence
  source 1..11951: mol_type = other DNA

SEQ ID NO: 24 - DNA, 11978 bp, synthetic construct
  misc_feature 1..11978: note = constructed sequence
  source 1..11978: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaacatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacaggttggtgtacactagcggccgccaccatgatggacctcgaactgccgccgcctggcctcccaagccaacaggatatggacctgattgacatcctgtggcggcaggacattgatctgggtgtcagccgcgaggtgttcgatttctcgcaacgccggaaggaatacgaactcgagaagcagaagaagctcgagaaagagcggcaggaacagctccagaaggaacaggaaaaggccttcttcgcacaacttcagctggacgaggaaaccggcgaattcctgcctattcaaccagcccagcacatccagagcgaaacctccggcagcgccaactattcccaagtggctcacatcccgaagtccgacgccctgtactttgacgattgtatgcagctgctggcacagaccttccccttcgtcgatgataacgaggtgtcctccgcgacgtttcagtcgctggtccccgacatccccggtcatatcgagagccctgtgttcatcgccaccaaccaggctcagtcccccgaaacctcagtggcacaagtggcgccggtggacttggacggcatgcagcaagacatcgaacaagtctgggaggagcttctgtccatccccgagctgcaatgcctcaacatcgagaatgacaagctcgtggagactactatggtcccgtccccggaagctaagctgaccgaggtcgacaactaccatttctactcctcaatcccctccatggaaaaggaagtcggaaactgctcgcctcatttcctcaacgccttcgaggactccttctcgtcaattctgtccactgaggaccccaaccagctgaccgtcaattccttgaactcggatgccactgtgaacaccgacttcggcgacgaattctacagcgcgttcatcgccgaaccgagcatctcgaactccatgccctcgcccgccaccttgtcacattccctgtctgagctgctgaacgggccgattgacgtgtcagacctgagcctgtgtaaagccttcaaccagaatcacccggagtcgactgccgaattcaacgactcggactccgggatctcactgaacactagccctagcgtggcctcgcccgaacactccgtggagtccagctcctatggcgatactcttctgggtctgtccgactccgaagtggaagaactggactctgcccccggaagcgtgaaacagaacggacctaagaccccagtgcactcctccggggatatggtgcagccgttgtcaccgagccaggggcaatccacccacgtgcatgacgctcagtgcgagaacacccccgagaaagaactcccagtgtcccccggacaccgaaagaccccgtttaccaaggacaagcactcctcacggctggaagcacaccttactcgggatgaactcagagccaaggccctccacattcctttccccgtggagaagattatcaatctccctgtggtggatttcaacgagatgatgagcaaggaacagttcaacgaagcgcagctggcgctgatcagggacatcaggcgcagaggaaagaacaaagtggccgcccaaaactgccggaagagaaagctcgaaaacatcgtggagctcgaacaggacttggaccacctgaaggatgaaaaagaaaagctgctgaaggagaagggagagaacgacaagtccctccatctgctgaagaagcagctgagcacactgtacctcgaagtgttttccatgctgcgcgatgaggatggaaagccgtactccccgtccgaatactcgctgcaacagacgcgcgacggaaacgtgttcctcgtgccaaagtccaagaagcctgacgtgaagaagaactacccatacgatgttccagattacgcttgaagtactgcggatcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 25 - DNA, 11853 bp, synthetic construct
  misc_feature 1..11853: note = constructed sequence
  source 1..11853: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagccacccacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaaggcaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacaccctgtacactagcggccgccaccatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactgaagtactgcggatcctgcaggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 26 - DNA, 11880 bp, synthetic construct
  misc_feature 1..11880: note = constructed sequence
  source 1..11880: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagccacccacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaaggcaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacaccctgtacactagcggccgccaccatgatggacttggagctgccgccgccgggactcccgtcccagcaggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggtgaatttctcccaattcagccagcccagcacatccagtcagaaaccagtggatctgccaactactcccaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactacccatacgatgttccagattacgcttgaagtactgcggatcctgcaggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 27 - DNA, 11853 bp, synthetic construct
  misc_feature 1..11853: note = constructed sequence
  source 1..11853: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagccacccacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaaggcaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacaccctgtacactagcggccgccaccatgatggacctcgaactgccgccgcctggcctcccaagccaacaggatatggacctgattgacatcctgtggcggcaggacattgatctgggtgtcagccgcgaggtgttcgatttctcgcaacgccggaaggaatacgaactcgagaagcagaagaagctcgagaaagagcggcaggaacagctccagaaggaacaggaaaaggccttcttcgcacaacttcagctggacgaggaaaccggcgaattcctgcctattcaaccagcccagcacatccagagcgaaacctccggcagcgccaactattcccaagtggctcacatcccgaagtccgacgccctgtactttgacgattgtatgcagctgctggcacagaccttccccttcgtcgatgataacgaggtgtcctccgcgacgtttcagtcgctggtccccgacatccccggtcatatcgagagccctgtgttcatcgccaccaaccaggctcagtcccccgaaacctcagtggcacaagtggcgccggtggacttggacggcatgcagcaagacatcgaacaagtctgggaggagcttctgtccatccccgagctgcaatgcctcaacatcgagaatgacaagctcgtggagactactatggtcccgtccccggaagctaagctgaccgaggtcgacaactaccatttctactcctcaatcccctccatggaaaaggaagtcggaaactgctcgcctcatttcctcaacgccttcgaggactccttctcgtcaattctgtccactgaggaccccaaccagctgaccgtcaattccttgaactcggatgccactgtgaacaccgacttcggcgacgaattctacagcgcgttcatcgccgaaccgagcatctcgaactccatgccctcgcccgccaccttgtcacattccctgtctgagctgctgaacgggccgattgacgtgtcagacctgagcctgtgtaaagccttcaaccagaatcacccggagtcgactgccgaattcaacgactcggactccgggatctcactgaacactagccctagcgtggcctcgcccgaacactccgtggagtccagctcctatggcgatactcttctgggtctgtccgactccgaagtggaagaactggactctgcccccggaagcgtgaaacagaacggacctaagaccccagtgcactcctccggggatatggtgcagccgttgtcaccgagccaggggcaatccacccacgtgcatgacgctcagtgcgagaacacccccgagaaagaactcccagtgtcccccggacaccgaaagaccccgtttaccaaggacaagcactcctcacggctggaagcacaccttactcgggatgaactcagagccaaggccctccacattcctttccccgtggagaagattatcaatctccctgtggtggatttcaacgagatgatgagcaaggaacagttcaacgaagcgcagctggcgctgatcagggacatcaggcgcagaggaaagaacaaagtggccgcccaaaactgccggaagagaaagctcgaaaacatcgtggagctcgaacaggacttggaccacctgaaggatgaaaaagaaaagctgctgaaggagaagggagagaacgacaagtccctccatctgctgaagaagcagctgagcacactgtacctcgaagtgttttccatgctgcgcgatgaggatggaaagccgtactccccgtccgaatactcgctgcaacagacgcgcgacggaaacgtgttcctcgtgccaaagtccaagaagcctgacgtgaagaagaactgaagtactgcggatcctgcaggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 28 - DNA, 11880 bp, synthetic construct
  misc_feature 1..11880: note = constructed sequence
  source 1..11880: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagccacccacaagccagttcctgtccctgaggacttggctcagggactctgggaatgtggtagacatggggtggccccaccaaatgcatccttatgggaacctgctccctgggagccatgaaaagagcgtggacttcgaggtggggccacaggaagtggtcaggtccatctcaggggacctgctgcccatccacactgctggccaggaaatggggggcaattcatgcctcctcagcaccttcagcactgggcggctcaaagaaggcaagggactattctggggtcacacagcatgcagccagaggccaaggcatgaggaagtccttcatttccccacccccacccacctcagatcctccaaccggtttcatggcagcccagggtccagcggcatccaggatgctggtgggtagctgcacagcccaggccgcgggaggttggctgctctcacctaacaggcctatgtggccctgacccctacctaggaagctggggacaatggccaaggcgcctcccctctctgtgcctgtctgtccaggtgcagcatagacacagcacccctggggccaagagcacccagccagggctgcccccatgggtgggcagggcagtaaatgaatgagggacaggttgggaggtggccagccccctccagcccatggagggcacggggcaggagagctgggctgagccagcaggagcccagggagcctggtctctgccttcctatcctggaggaaggtgaggctgaacctccttccctccctccctccctccccgcccccactgcacgcagggctggctgggctccagctggcctccgcatcaatatttcatcggcgtcaataggaggcatcggggacagccgctgcggcagcactcgagccagctcaagcccgcagctcgcagggagatccagctccgtcctgcctgcagcagcacaaccctgcacaccctgtacactagcggccgccaccatgatggacctcgaactgccgccgcctggcctcccaagccaacaggatatggacctgattgacatcctgtggcggcaggacattgatctgggtgtcagccgcgaggtgttcgatttctcgcaacgccggaaggaatacgaactcgagaagcagaagaagctcgagaaagagcggcaggaacagctccagaaggaacaggaaaaggccttcttcgcacaacttcagctggacgaggaaaccggcgaattcctgcctattcaaccagcccagcacatccagagcgaaacctccggcagcgccaactattcccaagtggctcacatcccgaagtccgacgccctgtactttgacgattgtatgcagctgctggcacagaccttccccttcgtcgatgataacgaggtgtcctccgcgacgtttcagtcgctggtccccgacatccccggtcatatcgagagccctgtgttcatcgccaccaaccaggctcagtcccccgaaacctcagtggcacaagtggcgccggtggacttggacggcatgcagcaagacatcgaacaagtctgggaggagcttctgtccatccccgagctgcaatgcctcaacatcgagaatgacaagctcgtggagactactatggtcccgtccccggaagctaagctgaccgaggtcgacaactaccatttctactcctcaatcccctccatggaaaaggaagtcggaaactgctcgcctcatttcctcaacgccttcgaggactccttctcgtcaattctgtccactgaggaccccaaccagctgaccgtcaattccttgaactcggatgccactgtgaacaccgacttcggcgacgaattctacagcgcgttcatcgccgaaccgagcatctcgaactccatgccctcgcccgccaccttgtcacattccctgtctgagctgctgaacgggccgattgacgtgtcagacctgagcctgtgtaaagccttcaaccagaatcacccggagtcgactgccgaattcaacgactcggactccgggatctcactgaacactagccctagcgtggcctcgcccgaacactccgtggagtccagctcctatggcgatactcttctgggtctgtccgactccgaagtggaagaactggactctgcccccggaagcgtgaaacagaacggacctaagaccccagtgcactcctccggggatatggtgcagccgttgtcaccgagccaggggcaatccacccacgtgcatgacgctcagtgcgagaacacccccgagaaagaactcccagtgtcccccggacaccgaaagaccccgtttaccaaggacaagcactcctcacggctggaagcacaccttactcgggatgaactcagagccaaggccctccacattcctttccccgtggagaagattatcaatctccctgtggtggatttcaacgagatgatgagcaaggaacagttcaacgaagcgcagctggcgctgatcagggacatcaggcgcagaggaaagaacaaagtggccgcccaaaactgccggaagagaaagctcgaaaacatcgtggagctcgaacaggacttggaccacctgaaggatgaaaaagaaaagctgctgaaggagaagggagagaacgacaagtccctccatctgctgaagaagcagctgagcacactgtacctcgaagtgttttccatgctgcgcgatgaggatggaaagccgtactccccgtccgaatactcgctgcaacagacgcgcgacggaaacgtgttcctcgtgccaaagtccaagaagcctgacgtgaagaagaactacccatacgatgttccagattacgcttgaagtactgcggatcctgcaggcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcctgcagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt